SEQUENCE LISTING (84 sequences)

SEQ ID NO: 1 - DNA, 97 bp, Artificial Sequence
  Description of Artificial Sequence Synthetic oligonucleotide
acaagcggcacattgtgcctattgcgaattaggcacaatgtgcctaatctaacgtcatgccagccacaacggcgaggcgccaagaaggatagaagcc

SEQ ID NO: 2 - DNA, 35 bp, Artificial Sequence
  Description of Artificial Sequence Synthetic oligonucleotide
tttacagctagctcagtcctagggactgtgctagc

SEQ ID NO: 3 - DNA, 155 bp, Artificial Sequence
  Description of Artificial Sequence Synthetic polynucleotide
gatttttttcgggtgaggttgcgggctgttcggtaggtttataaacactgctatccaaagctatggacacgctcggctacgagaacagttggcgtgatggcctctagcaattagattgttatgcgacatccgcagacttggcagggagcgcacct

SEQ ID NO: 4 - DNA, 35 bp, Artificial Sequence
  Description of Artificial Sequence Synthetic oligonucleotide
tttacggctagctcagtcctaggtatagtgctagc

SEQ ID NO: 5 - DNA, 199 bp, Artificial Sequence
  Description of Artificial Sequence Synthetic polynucleotide
atccgagggatacgggccttgtcagcacggtgttgctaatgagagcctttgcccgggcaatagtacgggcagtgtgtagcggattgaaagacgctgaatcactgacaggcatgaagactatcgatagagtctgatagtgtcgccgccgcacagcggatagagtccacagtcattgaagtgttaatccgcgatcaagctc

SEQ ID NO: 6 - DNA, 216 bp, Artificial Sequence
  Description of Artificial Sequence Synthetic polynucleotide
gacctagcttttatagcgggtttcgtggtttatagcccattgaaaaaaatctcacatctatatcacaggtgtgcactcgttcccgaaaggttctgagtctacttgatcaagtattgaaataccatcgtaaaggaaaaagacatgtctattcgtgatagcgaaaacaacaacggccaacagcagcagaccgcgcaaactgccgcccccgccccgcaa

SEQ ID NO: 7 - DNA, 182 bp, Artificial Sequence
  Description of Artificial Sequence Synthetic polynucleotide
ttcaatttaagtagtaacgaggtcagcccggaatctttgggtattcttaaggtatttctgactcagtgtggttgggacagcttcactgtacattgcactggatttgttaatttcttataccggggcaccatgggcagcaaatcgtgttacgaattccgtctaaccaataagcgagctaaata

SEQ ID NO: 8 - DNA, 105 bp, Artificial Sequence
  Description of Artificial Sequence Synthetic polynucleotide
cgcggaacccctatttgtttatttttctaaatacattcaaatatgtatccgctcatgagacaataaccctgataaatgcttcaataatattgaaaaaggaagagt

SEQ ID NO: 9 - DNA, 161 bp, Artificial Sequence
  Description of Artificial Sequence Synthetic polynucleotide
cttcaagaattcgtattgaccccatagacagcttcgtcgacgcccgtcccggcccccttgggcttgccggacggcttatgtcatgatggcgccaccctcgcaggttcaaggccggctttcttcctctatgaacaaatcccttgcgctgactacgtaatcac

SEQ ID NO: 10 - DNA, 207 bp, Artificial Sequence
  Description of Artificial Sequence Synthetic polynucleotide
cttcaagaattcggggtattcctgatcctgcgccgctagcgccgcgcacggccactaggcccgcgccgatagccagtcgcgctcccggctggcacactactcccatttccgccggaaacgcgcgcaacgtaccggcaacgaacgtggaaagaccatgaaagactggctggatgagattcactggaacgccgtgacctacgtatgcac

SEQ ID NO: 11 - DNA, 59 bp, Artificial Sequence
  Description of Artificial Sequence Synthetic oligonucleotide
gaaaattattttaaatttcctctagtcaggccggaataactccctataatgcgacacca

SEQ ID NO: 12 - DNA, 50 bp, Artificial Sequence
  Description of Artificial Sequence Synthetic oligonucleotide
tttacagctagctcagtcctagggactgtgctagcattaaagaggagaaa

SEQ ID NO: 13 - DNA, 140 bp, Artificial Sequence
  Description of Artificial Sequence Synthetic polynucleotide
gcacaaggcgttgtttgcatggctgaatttgatgaatactttttagattatgatgatatgatagaatggtctaaaagatacattaaaaggaatcttttgtgaagaattttaaactaaaccgagttaggtatcaaaatata

SEQ ID NO: 14 - DNA, 147 bp, Artificial Sequence
  Description of Artificial Sequence Synthetic polynucleotide
tataaataataatatgaattgggtgtcggaataataagttaaccgaacaattctatgtggtagtctacaactgagagatctgtcgaaagaagatgaaattcagaagaacgtgactaccgagttttaatctctaacgagaatttttaa

SEQ ID NO: 15 - DNA, 100 bp, Artificial Sequence
  Description of Artificial Sequence Synthetic polynucleotide
gtcgcaatgatgtacgaaggctggaagggtgccaaaaagtttagctaagggcttcggccctttttggataataaaattttaacgtaattgaggataatgt

SEQ ID NO: 16 - DNA, 56 bp, Artificial Sequence
  Description of Artificial Sequence Synthetic oligonucleotide
tttacagctagctcagtcctagggactgtgctagcattaaagaggagaaatgatat

SEQ ID NO: 17 - DNA, 150 bp, Artificial Sequence
  Description of Artificial Sequence Synthetic polynucleotide
accctggagacctagatgctgaagaagtcgaccagctcatcagaaacgtagaaatcttgattgccgaacatgtcaagatttgatataataattgggtaagtttgagaagaaacactgacgttttacttgcttacctaataggagaaatca

SEQ ID NO: 18 - DNA, 150 bp, Artificial Sequence
  Description of Artificial Sequence Synthetic polynucleotide
gcgatgcgtactacgttactcagcgtctgaacctgatgcgtttcttcgagaacggcgtagttgctggtgcttacgctgcttaatttggctatactgccgactaaaggagagcttcggctctccttttttattgggtaaaatatgcaattc

SEQ ID NO: 19 - DNA, 120 bp, Artificial Sequence
  Description of Artificial Sequence Synthetic polynucleotide
tctttatctaaattctacttgacatttgtctcaaagtatgatccgagcaataactataaaattttattgcaacttcctccaataaagggtataattataaatggttagaggaggttctta

SEQ ID NO: 20 - DNA, 165 bp, Artificial Sequence
  Description of Artificial Sequence Synthetic polynucleotide
atgcgcgggcgcacacgaataatataaaatggggtagataggcaaataaaattttccggtttttattgttttgatgatccagatcaagttactgttgattataataaaataaggttgacatagctgtgtctatttgatataatatatgcctaactggaggaatat

SEQ ID NO: 21 - DNA, 125 bp, Artificial Sequence
  Description of Artificial Sequence Synthetic polynucleotide
agctaaggctgaagccaaagcaaagtaagtttaaagatttaattgctgttaaaccttaggtgtataattactttactttctaaacaacgtgagaaaaatataatggtgactaatggtaaatatta

SEQ ID NO: 22 - DNA, 60 bp, Artificial Sequence
  Description of Artificial Sequence Synthetic oligonucleotide
aaggggttttctttttcatagaaccctatataatgtcagtgattaaacaaagaggtgaat

SEQ ID NO: 23 - DNA, 300 bp, Artificial Sequence
  Description of Artificial Sequence Synthetic polynucleotide
agcagccctgcgcgagctggagttcgccgacggctgcgccgccatcgcccgtcgcctgcagccatatctggtgcagatgccacgcaagggttatcaggcattgcgggaagccggtgcgatccaggcggcggcaggtacgcgttatggtgagcagtttatggcgttggtcaaccctgatctgtatcaccaccaattcgggttgcactgggataatccggcctttgtcagcagcgagcggctatgttggtagtcgggacgcgcaacagcggcctggcctggatgatgtgaaagggagggccg

SEQ ID NO: 24 - DNA, 50 bp, Artificial Sequence; identical to SEQ ID NO: 12
  Description of Artificial Sequence Synthetic oligonucleotide

SEQ ID NO: 25 - DNA, 59 bp, Artificial Sequence; identical to SEQ ID NO: 11
  Description of Artificial Sequence Synthetic oligonucleotide

SEQ ID NO: 26 - DNA, 139 bp, Artificial Sequence
  Description of Artificial Sequence Synthetic polynucleotide
atttacgatattaatgtatcacgagctccgtcaatggttactattccagccgaagaactagatcgtcttcagaaaattgaagagcttctttgggaaattgaatctgatttgccatcaggattagaatcctggattgatt

SEQ ID NO: 27 - DNA, 250 bp, Artificial Sequence
  Description of Artificial Sequence Synthetic polynucleotide
aaatatagcattgatgatgcttttaattatgaagaagaattcgaaacggaaattcaattcttaatgaaaaagtataatctcaagcgtcaggatattcgtatcctggccgaccacccatgcggtgaagatgttctttatattaaaggaaaatttgccggatatcttgatgaatatttttattctaaagatatgggcattgatatgcatatgagagttgtataaatagatatataattcagaggagacaatc

SEQ ID NO: 28 - DNA, 200 bp, Artificial Sequence
  Description of Artificial Sequence Synthetic polynucleotide
aataacatagaaaagatttatcgtctttgtgataaaattgaaaaagaaaagaaatatctattttgtctatggcctattgttgacggaagagtaggcctagatgttcttgattatgaaacagaagacaaagtagatggcgcaacttttgataacgctttggatgttattgattggctcgaagaaaattatgtgaggtaaat

SEQ ID NO: 29 - DNA, 149 bp, Artificial Sequence
  Description of Artificial Sequence Synthetic polynucleotide
aaaacatttaaaattgtcgtagaattttataacggcgaaggtgtagtccatcttaaagcggccaaccagtttgatgcggtaagagtatattgtcaatgctttgaaagttctaaacaagctataaaaattaaaagtgttgaggaaaccaa

SEQ ID NO: 30 - DNA, 248 bp, Artificial Sequence
  Description of Artificial Sequence Synthetic polynucleotide
gatttatttgagatgttagaagataatcattctacgaataaccagaatgattctagtgattataagaaagagtaccgtatagtattacagaattatggaattgaagccccagatgctcttctagaagaactagcttcataccatcttgaccctccgccctgggctccctgggcaaaataattcaaaaagttgtttactttcctttctaacgatgatatgatagcttctgaagtatacgggaggctatc

SEQ ID NO: 31 - DNA, 300 bp, Artificial Sequence
  Description of Artificial Sequence Synthetic polynucleotide
taatgatttagattctagcggaaatccttcacacactgccggtggaacagttggaacaacgtcagtaacgcttgaaaatgcaaatcttcctgcgactaaaactgacgaaagagttttaattgaagatgaaaatggatcagttattattggaggttgtcaatatgacccagatgaaactggtcctatatatacaaaatatcgtgaagactatgcaacaacaaactcttcacatactcctcctactaatattagtaatattcaaccgtctattactgtataccgttggataaggattgcata

SEQ ID NO: 32 - DNA, 300 bp, Artificial Sequence
  Description of Artificial Sequence Synthetic polynucleotide
ggaatgcatgttggtggtgttcaggcacaacaaatgtcataccataaacatgctggtggttggggtgaatataacagaagtgaaggcccatttggcgcgtcagtttatcaaggatatcttggaactagaaaatattccgactgggataatgcttcatacttcactaatgatggatttgaattaggtggaccgagagatgctcatggtacacttaatcgtgaaggattaattggttatgaaactagaccatggaatatatcattaaactatattattaaagttcattactaaggattacaa

SEQ ID NO: 33 - DNA, 300 bp, Artificial Sequence
  Description of Artificial Sequence Synthetic polynucleotide
aacttacttcacttataaatggaaataatcctgacggatcaactgttgaagaacgaggattaactaattctataaaaacgaatgaaaccaacattgcggcagtcacacatgaagtaaatacagctaaagacaatatatcctctttacagagcagcgttcaagctctacaagaagcaggttatattcctgaagctccaaaagatggccaagcttacgttcgtaaagacggcgaatgggtactactttctacctttttatcaccagcataacatggggccgcaaggccccaaaggattttaa

SEQ ID NO: 34 - DNA, 300 bp, Artificial Sequence
  Description of Artificial Sequence Synthetic polynucleotide
atagttggtcattatctgaacgatacaataatccagaccataatttagtaggtcgtgttgtcggtcaagatccaaatgttaagcaaggtgcttataataatcgttgggtgaaagactatgcaacagctttagctaaagaattgaatggtcaaattttagcgcgtcaccagggaatgatgcttccaggcggtgttacgattgatggacaacgtttaatagaagaagctcgattagaaaaagaagcattacgcgaagaattatacttacttgaccctccatttggaatttttgtaggttaat

SEQ ID NO: 35 - DNA, 300 bp, Artificial Sequence
  Description of Artificial Sequence Synthetic polynucleotide
aacaaatgctaatggacgcagccaagatttttcttgagacggcaaagaatgctgattctcctcgtcacatggaagtatttgcaactcttatggggcaaatgactacgacgaacagagaaatactgaagcttcacaaagacatgaaagatattacatctgaacaggttggcaccaaaggcgctgttcctacaggtcaaatgaatattcagaatgcgacagtattcatgggttcaccaacagaattaatggacgaaattggtgatgcttacgaggctcaagaagctcgtgagaaggtgataa

SEQ ID NO: 36 - DNA, 300 bp, Artificial Sequence
  Description of Artificial Sequence Synthetic polynucleotide
agatgcagttaaaaataagacgttatttgagaaaatgacatcgagtttaactaacgttcttgtagtttcaaacccgacaatttggatggtgaaaaactttggtgcaacatctaagtttgatggaaaaacggaaatattcggtccatgtcaaatccagagtatcagatttgataaaacacctaatggtaactttaacggattagctattgctccaaatctccctagtacatttactctcgagattactatgagagaaattatcacgttaaaccgtgcttctttatatgcggggacttttta

SEQ ID NO: 37 - DNA, 300 bp, Artificial Sequence
  Description of Artificial Sequence Synthetic polynucleotide
taaggtgacttatacttgtaatctatctaaacgggggacctctctagtagacaatcccgtgctaaattgtaggactaccctttaataatttcatcaggattagttacttaccgtgtaaaatctgataaatggaattggttctacataaatgcctaacgactatccctttggggagtagggtcaagtgactcgaaacgatagacaacttgctttaacaagttgaagatatagtctgatctgcatggtgacatgcagctggatataattccggggtaagattaacgaccttatctgaacata

SEQ ID NO: 38 - DNA, 300 bp, Artificial Sequence
  Description of Artificial Sequence Synthetic polynucleotide
gtttggtaacacacttgattcgctttaccaagattggattacttatccaacgaccccagaagcacgtaccactcgctggacacgtacatggcagaaaaccaaaaactcttggtcaagttttgttcaggtatttgacggaggtaaccctcctcaaccttcagatataggagcgatcccatctgataatggaataataggtaatcttactattcgtgatttcttacgaattggtaatgttcgcattattcctgacccagtgaataaaactgttaaatttgaatgggttgaataagaggtatt

SEQ ID NO: 39 - DNA, 287 bp, Artificial Sequence
  Description of Artificial Sequence Synthetic polynucleotide
gccttaaaagcaacggcactatttgccatgctaggattagcgtttgctttatctccaccaattgaagcgaatgtcgatcctcattttgataaatttatggaatctggtattagacacgtttatatgctttttgaaaataaaagcgtagaatcatctgaacagttctatagttttatgcgaacgacttataaaaatgacccgtgctcttccgattttgaatgtatagagcgaggcgcggagatggcacaatcatacgctagaattatgaacattaaattggagactga

SEQ ID NO: 40 - DNA, 300 bp, Artificial Sequence
  Description of Artificial Sequence Synthetic polynucleotide
cggtctgttatttaaacgacgattatgattatcttggcgtttatagtttaagtgatgcacggtttaaacgtaatttacaaaagtcaaatttattttatattgatactacggtaaaatttcagggcaagaaatatttctttactcttatagttgattctgaaacgaagcatgagaataaacgtattcttagtaaaaagaatatcttaactattgttgatgatctttttgataaattcgtagaaaatcccaattttgaaagcgatttattactagaaaaatttgttaaggaatgtagagaat

SEQ ID NO: 41 - DNA, 300 bp, Artificial Sequence
  Description of Artificial Sequence Synthetic polynucleotide
tgaaatcgccaagaaagtagttgagttagatgatgctcgtcaagaacttgcggttaaattggaatatatccgtgaaactcgtgcagcaaatgcccttggaattagtactgccgatgatgtagttgaaattgcagcactgactaaggttgatattgaagatacccttgctcgagttgaaacctttaacggtaatatttctggggttgaaactacctctgccgatgttcaggaatacattaattctctgaaataatgataaggggcttcggccccttatacttggagtaaataggaatgaaa

SEQ ID NO: 42 - DNA, 297 bp, Artificial Sequence
  Description of Artificial Sequence Synthetic polynucleotide
actgctataactccgcaagaatacatggcgtctcttaaagaaaaatataatctttctgcaacagaaacacttttcgatttaccagaaaaccttcaactaaaatttcaggtagaatttcaaaaattagttcacccagaacaaaaacactttactgcagtcgttaagtcaattaatgcagatggattgacaattttcacccgacaaatagtactaatttaagcaaggggcttcggccccttaattggagtataatatatcaagagcctaataactcgggctataaactaaggaatatct

SEQ ID NO: 43 - DNA, 300 bp, Artificial Sequence
  Description of Artificial Sequence Synthetic polynucleotide
ttgagcgaaagtactaaagatctgactgagtctcaaaaagaaaaagtctctgctctggtcgaaggtatggattattcagatgcattctcaagtaaattgagtgcaatcgtagaaatggtgaagaaatctaataaagatgaaagcactattactgagagtataaatactcctgatactgaagcagccggactgaatttcgtcactgaagctgtagaagataaatctgcacaaggtgcagaagatattgtaagtgtatatgcgaaagtcgcatctcgtttctaattttaaaggttaacacaa

SEQ ID NO: 44 - DNA, 300 bp, Artificial Sequence
  Description of Artificial Sequence Synthetic polynucleotide
tgtattagtaggtcctgtgacagctgtatcatttataatcctaatgattattggaatagttatagatgttactactgatattgaatcagacgcagtatttctgttagtattaattcttccattagtagttccatttttattagtacctgtaaattgggtaggatactggtatcaaggaagacattatcgtaaacgcgtatgcgaatggaaagctcagtgtaaaaagattaaaaaggaacatcagcttaaacttgctgcgtatgaatttaatgaaattatgaaatttgttaaggaatcacg

SEQ ID NO: 45 - DNA, 300 bp, Artificial Sequence
  Description of Artificial Sequence Synthetic polynucleotide
ctattgtagtgtcaaggtcaggtgcttattctgaaatgacttataggaatggctatgaagaagctattcgtcttcaaactatggcgcaatatgatggctatgctaaatgttctactgtcggtaattttaacttgactcctggtgttaaaattatttttaatgatagtaaaaaccaatttaaaacagaattttacgttgatgaagttatccatgaattatcaaataataattcagtaactcatctatatatgttcactaatgcaacgaaactggaaacaatagacccagttaaggttaaaa

SEQ ID NO: 46 - DNA, 300 bp, Artificial Sequence
  Description of Artificial Sequence Synthetic polynucleotide
atgctcgtgaatttcttgacgaagaaaccggcgagatgattcgcgaagaaaaatcttggcgtgcaaaagatactaactgcactacattctggggtcctttatttaagcatcaaccattccgagatgctattaaacgtgcttatcagttaggtgctattgatagtaatgaaattgttgaagctgaagttgatgaattgattaactcaaaggttgaaaaatttaaatctccagaaagtaaaagtaaatcagctgctgatttagaaactgacctcgaacagttaagtgatatggaagaattta

SEQ ID NO: 47 - DNA, 300 bp, Artificial Sequence
  Description of Artificial Sequence Synthetic polynucleotide
taagtgatgaagctcattttaattatctgatggctgctgttcctcggggtaaaagatatggtaaatgggcaaaactggttgaagattccaccgaagtattgattattaagttacttgctaagcggtatcaagttaatacaaatgatgcaattaactataaatcaattcttactaaaaatggaaaactatctttagtattaaaagaactaaaaggtttagtcacgtatgattttttgaaagaagtgactaagaacgtaaaagaacagaaacaactcaaaaaactagcattggaatggtaaa

SEQ ID NO: 48 - DNA, 300 bp, Artificial Sequence
  Description of Artificial Sequence Synthetic polynucleotide
atcgtaattaacggttttaataaagtagaagattctgctctgacccgtgttaaatattctttgactcttggtgattatgatggtgaaaatacatttaatttcattatcaatatggcaaatatgaaaatgcaaccaggaaattataaacttctgctctgggcaaaaggtaaacaaggtgctgctaaatttgaaggtgaacacgcgaattatgtggtagctcttgaagctgattctacccacgatttttaataaagggcttcggccctttataatttacactaaaacttgaatgaggaaatt

SEQ ID NO: 49 - DNA, 300 bp, Artificial Sequence
  Description of Artificial Sequence Synthetic polynucleotide
tttcagttaaaaatctgcatcacgttgttttagcacacggtgttaaatctaaaattattgtattgcaaacaataggtcgcgtattacgtaaacatggttctaaaacaatcgcaacagtctgggacctcatagatgactgtggtgtcaagccaaaatctgctaatactaaaaagaaatatgttcacttgaactatcttttaaaacacggcattgatcgtattcagcgctacgcagatgaaaaatttaattacgtaatgaaaacagtcaatttataagggcttcggccctttggagaaaaag

SEQ ID NO: 50 - DNA, 300 bp, Artificial Sequence
  Description of Artificial Sequence Synthetic polynucleotide
tataataaattttggtcagaagacgaagaaaaagaccttttaaatcttttagactctttaaatgacagaggaataaaatttggactgtcgaatgttttagagcaccacggaaaggaaaatactcttcttaaagaatggtctaaaaaatacactgttaagcatcttaataaaaaatacgtctttaacatatatcattccaaagaaaagaatggaactgatgaagtatatatttttaattaattgcttatatattcaaatggtataattatttaacttattaatgaattgaaaggaaaaata

SEQ ID NO: 51 - DNA, 300 bp, Artificial Sequence
  Description of Artificial Sequence Synthetic polynucleotide
caaagcatcacgcttatggaatgttccaaaattatttgcctactatgcgagcgagagtcaaggaacttggttataatatgaccgatgctgaaataaaaagaatgttgaataaacggtccaattcagcttcctgggcgtacattgaactttcttattggttaaatatacataagggcgatataagaaaagcaatatcctcttataattcgggatggaatgttaaagctggttctaaatatgcttctgaagtcctagaaaaggctaattaccttaaaaataataaacttttggaaatagtaa

SEQ ID NO: 52 - DNA, 300 bp, Artificial Sequence
  Description of Artificial Sequence Synthetic polynucleotide
aatcaaagaaactgaatcagcatttcgtttagcttttgccaaggcacatttcattaagaaagtaatttcaggtgaaattgttgtacaaggtaaaactcgcaaagaactgaccgaagaactttctaaaattgatatgtattcttcttatgttgataaactagttggaatgaacatttttcatatgacttccgacgaagcaaagaaacttgctgaagaagctaaagccaaaaaagaagaaaacgaatattggaaaactactgatgtagttacagaatacaccaaagatttagaggaaatcaa

SEQ ID NO: 53 - DNA, 235 bp, Artificial Sequence
  Description of Artificial Sequence Synthetic polynucleotide
gaaataactaaagatcagttttatcttcttcaagataaagtaagcgaaatttatgaaattgcttatagcaaaaatcgtgaaactgtaaaaatagaatctagtaagttgatgcttcaattagaagaaattgaacgagatttaattgcgttagaattcttttgtggtgaagtgaaaactgttacaatcaatgattatattttaggcgaaattagctatctttataaggcgattatta

SEQ ID NO: 54 - DNA, 300 bp, Artificial Sequence
  Description of Artificial Sequence Synthetic polynucleotide
tcggagggggaagaagaaagaaaagaacgtctttttaatgaatctcttaagataattaaatctgccatggaaaatgttatccaggagattgtcattaaactagaagatggttctacacacattgtgtatgtgacaaaattagattgggttgatggaaaagtcgtaatggactttgctgttcttgaccaagaaagaaaagctgagttagctcctcatgtagaaaaatgtattacaatgcaactacaagatgcatttaataaaaggtcaaagaaaaagtttaaattcttttaaggagtaagt

SEQ ID NO: 55 - DNA, 195 bp, Artificial Sequence
  Description of Artificial Sequence Synthetic polynucleotide
gaatattcaactggacagcatttattaacttttcctgaaataaaacgatatattctgagaaataatttttctaatgaagagcatatagttactgaatctatgcttaggaatgcatttaaagcagaatatacaaaaataatgtccaatagaaatgaagcttggactgttactgattattatgactaaaggtgtatt

SEQ ID NO: 56 - DNA, 300 bp, Artificial Sequence
  Description of Artificial Sequence Synthetic polynucleotide
atttacgataaagttaagtctattaaagaccgtagtgttattaaacgtgaagttggtattattgctcaggaccttgaaaaggaattaccggaagctgtatctaaagttgaagttgatggatctgatgttctgacaatttctaactctgctgtgaatgctcttttaattaaggctattcaggaaatgagtgaagaaattaaagaattgaaaactcctttctttactaaaattgctcgcaaaattagtaaatattttaaattctaacaacaagggggaaatgccccctttggagtataaatt

SEQ ID NO: 57 - DNA, 300 bp, Artificial Sequence
  Description of Artificial Sequence Synthetic polynucleotide
gaactgtataagtttaacctatttttaggtaaaacggcagaaacttataaaaattggaacaaaggcggaaaagctccatggagtgatttttgggatgctaaatcacagtttagtaaagtgaaagcacttcctgcatcaacattccataaagcacagggcatgtctgtagaccgtgctttcatttatacgccttgtattcattatgcagatgctgaattggctcaacaacttctttatgttggtgttacccgtggtcgttatgatgtgttttatgtatgattaaatttgaggaagctattc

SEQ ID NO: 58 - DNA, 244 bp, Artificial Sequence
  Description of Artificial Sequence Synthetic polynucleotide
aaaattgttatggggtgcttagcagtttgtttagttgcattagcagcagttccatttgttagtgttgaaaatgatactcaacctgtgattgaatctagcaccgttattcacactaatggtaaaatatcagttaaaattgatgataatcttcatgtgaatactaatggaacgctgggtgttcaattaggtaatatctgtgtaagcactactggagtaattactacttgtatttgaggaaattatt

SEQ ID NO: 59 - DNA, 300 bp, Artificial Sequence
  Description of Artificial Sequence Synthetic polynucleotide
atgctggtattttatcactggttactaacgatcgtggtgctattgatgatgttcttgagtctctcaaaaataaagatgttaaacaactcagagctttagcaccaaaatatgcagctgattattcgtggttcgtaggtaaacttgccgaagaaatctattcacgtgtaactccgcagagtattattcgtatgtacgaaattgtcggcgaaaataatcagtatcatggtattgcagctaatactgaattgcatttagcttatcttttcattcagttagcatgtgaaatgcagtggaagtgat

SEQ ID NO: 60 - DNA, 300 bp, Artificial Sequence
  Description of Artificial Sequence Synthetic polynucleotide
taaagaaatagatggttatacttatgatattaatgacgtttatgtatgtcaaagattggaatttcaataccaaggaaatacattttattttagacctcctggaaaatttgaacaatttttaactgtaagcgatatgttatccaaatgcttgcttaaggtcaatgatgaagttaaagaaattaattttcttgagatgccagcatttgttttaaaatgggcaaatgatatttctacaactttagcaattcctggccctaatggtccaataaccggaattggcaatattattggattatttga

SEQ ID NO: 61 - DNA, 300 bp, Artificial Sequence
  Description of Artificial Sequence Synthetic polynucleotide
accagcagttaaaaaagaacttgcttctagatttgctaaaattgatgccacttatcaagagcttaagaaaaatcaacctgaggccaaacctgaaacttctgctaaatcaccagaagcaaaacaggtccaggtgattgaaaagaacaaagcacaacaagctcctgttcaacaagcatctccttcaatcaataatactaataatgttattaagaaaaatactgtcgttcataatatgacacctgtcacaagcacaactgctcctggtgtatttggtgcgacgggagttaattaaggaataat

SEQ ID NO: 62 - DNA, 300 bp, Artificial Sequence
  Description of Artificial Sequence Synthetic polynucleotide
tcatattgtagctaagatgtgtaatcttattccgggagatttgatattttccggtggtaatactcatatctatatgaatcacgtagaacaatgtaaagaaattttgcgtcgtgaacctaaagagctttgtgaactggtaataggcggattgccttataaattccgctatctttctactaaagaacaattggaatacattcttaaactcaggcctaaagatttcgttctcaaagattatcagtcccacggcgtcttgaaaggaaaaatggcggtgtaattttaatttaattgtgaggattt

SEQ ID NO: 63 - DNA, 300 bp, Artificial Sequence
  Description of Artificial Sequence Synthetic polynucleotide
gcttcctgatgcggttgaggagatgaaagtctttttagaaaatcagcttgcgaaatatgattgcgatgtgttcattaatcagactcaacctaatgttcatattaacaactgtaaatgctatatcatcgttaatcctttaacgggaaaacatcgtcttggaattagtaatccaaatcgtagcgcatcggatatggcagaagatgttgaggcatgctttaaaatttctaaatctccggctgaacatcatattttaattaacggtctttctcaagacgatattatagaggttattaaaacttt

SEQ ID NO: 64 - DNA, 300 bp, Artificial Sequence
  Description of Artificial Sequence Synthetic polynucleotide
aatgacttcaattgaatttactagtgccgttagaatagctctacaggaaatggttgtaaaatttattgcaattgattcatttgaagaccatcctaccataggaaataaaatacaagttaagtatttagataaccaagaacatatcttagaacaatactctgataaaggaattactttcaaacaggaaataatttctccttctaaacctgggtatggaacttggcaattattaggtgcgcaaactgttacgctagatagtcacacacaacctacagtattttattattttgagagagtagc

SEQ ID NO: 65 - DNA, 300 bp, Artificial Sequence
  Description of Artificial Sequence Synthetic polynucleotide
ttgctaataataaagataaagttctgtatcagtcctgtcatattcttcagaaaaaaggactatactatatcgttcattttaaagaaatgcttcgtatggatggtcgccaagttgaaatgacagaagaagatgaagttcgtcgtgattcgattgcatggctgttagaagattggggactgattgaaatcgttcctggtcaaagaacttttatgaaagatttaactaataatttccgagttatttcttttaaacaaaaacatgaatggaaactcgttcctaaatatacgattggtaattaat

SEQ ID NO: 66 - DNA, 300 bp, Artificial Sequence
  Description of Artificial Sequence Synthetic polynucleotide
ctcggctttctaaatcaaaacaaacctctgacgttaagctaacgattgtagcactcaaagctcgtattgatggttctcgtatagcagaaggcgctgaagttgttaaattgaacgttcttcttaaaggctctgattggaaaactgtgaaaaagttgtcagaagcagaaatgcaatatgatatgtgtgataaaattattcaaggtgtagagcggtatcaaaacttgtcttttattgataaacttaaactgaaaagaggatacccgttaaattgttcaatttttaaacttatccgaggataat

SEQ ID NO: 67 - DNA, 486 bp, Artificial Sequence
  Description of Artificial Sequence Synthetic polynucleotide
atgtctcgcggccaatacagccgcgaagacaataattgtactcacttcccagtggggcaatcccacatgctgttggagctgcgcacagccttcagtcaagtaaagacgttttttcaaactaaggatcaactggacaacatcttgttaacggatagccttatgcaggacttcaaaggttaccttggatgtcaagcgttaagcgagatgatccagttctatttagtggaggtgatgcctcaagcggagaaacatggtccagagattaaggaacacttaaatagccttggtgagaagctgaagacccttcgtatgcgcttacgtcgctgccatcgctttcttccctgtgaaaacaaatcgaaggcagtcgaacaagtgaaatccgatttcaataaactgcaagaccagggcgtgtataaagctatgaacgaatttgacatttttatcaactgtatcgaagcatatatgatgatcaagatgaaaagttaa

SEQ ID NO: 68 - protein, 161 aa, Artificial Sequence
  Description of Artificial Sequence Synthetic polypeptide
MSRGQYSREDNNCTHFPVGQSHMLLELRTAFSQVKTFFQTKDQLDNILLTDSLMQDFKGYLGCQALSEMIQFYLVEVMPQAEKHGPEIKEHLNSLGEKLKTLRMRLRRCHRFLPCENKSKAVEQVKSDFNKLQDQGVYKAMNEFDIFINCIEAYMMIKMKS

SEQ ID NO: 69 - DNA, 516 bp, Artificial Sequence
  Description of Artificial Sequence Synthetic polynucleotide
atggtcttcacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 70 - protein, 171 aa, Artificial Sequence
  Description of Artificial Sequence Synthetic polypeptide
MVFTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 71 - DNA, 19 bp, Artificial Sequence
  Description of Artificial Sequence Synthetic oligonucleotide
ctctaacgagaatttttaa

SEQ ID NO: 72 - DNA, 400 bp, Artificial Sequence
  Description of Artificial Sequence Synthetic polynucleotide
cttagccccgaccgaaaggttggggctttttggtataaatattagtatattaaatctacaaattaaaacaggaaataagatgaaatcatatactcaatttttaaatgaagcggtgttaaatgaagcatctagcaccgaaattcaagctgttgcaaaagctgccattgccgcgggtaaatattcctataaagatgcttctgatgaatcgcgattccagtttgcacgcgacatgaaagcggaaggatttacgggaaatgcagttagtatggcctggaaaagtttagttgctactggcgctgcttttgcaaaggcttcgggtaaacctgctcctaaagcagatcctaaagcggcacaagaaaaaaaatatcgttaaaggaattatcgctaaatatgaagctat

SEQ ID NO: 73 - DNA, 19 bp, Artificial Sequence
  Description of Artificial Sequence Synthetic oligonucleotide
ctatagactaaaggtcaca

SEQ ID NO: 74 - DNA, 35 bp, Artificial Sequence; identical to SEQ ID NO: 2
  Description of Artificial Sequence Synthetic oligonucleotide

SEQ ID NO: 75 - DNA, 35 bp, Artificial Sequence
  Description of Artificial Sequence Synthetic oligonucleotide
tttacggctagctcagtcctaggtacaatgctagc

SEQ ID NO: 76 - DNA, 35 bp, Artificial Sequence
  Description of Artificial Sequence Synthetic oligonucleotide
ttgacggctagctcagtcctaggtacagtgctagc

SEQ ID NO: 77 - DNA, 100 bp, Artificial Sequence
  Description of Artificial Sequence Synthetic polynucleotide
ccgacaaatagtactaatttaagcaaggggcttcggccccttaattggagtataatatatcaagagcctaataactcgggctataaactaaggaatatct

SEQ ID NO: 78 - DNA, 166 bp, Artificial Sequence
  Description of Artificial Sequence Synthetic polynucleotide
gttgataaagaaattaaaaagggacagtattatctcattaatggtaatgctgttcgtgttacttacgtaaatggttatgatgtttattatcttatacttaagttacataaacacatgatttgcgatcgtgctgcatttagttcagttgctaaggaaattaaactcc

SEQ ID NO: 79 - DNA, 140 bp, Artificial Sequence; identical to SEQ ID NO: 13
  Description of Artificial Sequence Synthetic polynucleotide

SEQ ID NO: 80 - DNA, 100 bp, Artificial Sequence
  Description of Artificial Sequence Synthetic polynucleotide
agcatactgtttacttagataactgatatcctctatgctttaagatagatcttcaaatattatgatataatagatctatgaattgagctaagaggtgaaa

SEQ ID NO: 81 - DNA, 100 bp, Artificial Sequence; identical to SEQ ID NO: 15
  Description of Artificial Sequence Synthetic polynucleotide

SEQ ID NO: 82 - DNA, 15 bp, Artificial Sequence
  Description of Artificial Sequence Synthetic oligonucleotide
attaaagaggagaaa

SEQ ID NO: 83 - DNA, 486 bp, Artificial Sequence; identical to SEQ ID NO: 67
  Description of Artificial Sequence Synthetic polynucleotide

SEQ ID NO: 84 - protein, 161 aa, Artificial Sequence; identical to SEQ ID NO: 68
  Description of Artificial Sequence Synthetic polypeptide

(19) United States
(12) Patent Application Publication
(10) Pub. No.: US 2023/0346861 A1
(43) Pub. Date: Nov. 2, 2023

(54) PHAGE DELIVERY OF ANTI-INFLAMMATORY PEPTIDES

(71) Applicant: Locus Biosciences, Inc., Morrisville, NC (US)

(72) Inventors: David G. OUSTEROUT, Morrisville, NC (US); Kurt SELLE, Morrisville, NC (US); Shaun STEELE, Morrisville, NC (US)

(21) Appl. No.: 17/736,854

(22) Filed: May 4, 2022

Related U.S. Application Data
(60) Provisional application No. 63/184,428, filed on May 5, 2021.
(60) Provisional application No. 63/184,527, filed on May 5, 2021.
(60) Provisional application No. 63/184,532, filed on May 5, 2021.
(60) Provisional application No. 63/309,789, filed on Feb. 14, 2022.

(51) Int. Cl. A61K 35/76; C12N 7/00

(52) U.S. Cl. CPC A61K 35/76; C12N 7/00; G01N 2500/00; C12N 2795/00032

(57) ABSTRACT

Disclosed herein are bacteriophage comprising an therapeutic peptide and methods for the use thereof.

### CROSS-REFERENCE

This application claims the benefit of U.S. Provisional Application No. 63/184,428, filed May 5, 2021, U.S. Provisional Application No. 63/184,527, filed May 5, 2021, U.S. Provisional Application No. 63/184,532, filed May 5, 2021, and U.S. Provisional Application No. 63/309,789, filed Feb. 14, 2022, each of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on May 3, 2022, is named 53240-741_201_SL.txt and is 41,997 bytes in size.

### SUMMARY

In certain aspects, disclosed herein is a method of producing a therapeutic peptide in a host organism, the method comprising: (a) contacting the host organism with a lytic bacteriophage comprising a nucleic acid encoding the therapeutic peptide; (b) producing the therapeutic peptide within one or more of a plurality of target bacterial cells present in the host organism; and (c) releasing the therapeutic peptide by lysis of the one or more target bacterial cells. In some embodiments the bacteriophage and the plurality of target bacterial cells co-exist in the host organism for at least about 1 day after contact. In some embodiments the therapeutic peptide is released for at least about 1 day after contact. In some embodiments a therapeutically effective amount of the therapeutic peptide is produced without eliminating the plurality of the target bacterial cells. In some embodiments a therapeutically effective amount of the therapeutic peptide is produced, and the plurality of target bacterial cells remains above the limit of detection for at least about 1 day after contact. In some embodiments detection is based on presence of colonies after plating on an agar plate. In some embodiments the target bacterial cells comprise E. coli, K. pneumoniae, R. gnavus, E. gallinarum, E. faecalis, E. faecium, or B. fragilis, or two or more thereof. In some embodiments the target bacterial cells comprise Enterobacteriaceae, Pasteurellaceae, Fusobacteriaceae, Neisseriaceae, Veillonellaceae, Gemellaceae, Bacteriodales, Clostridiales, Erysipelotrichaceae, Bifidobacteriaceae, Bacteroides, Faecalibacterium, Roseburia, Blautia, Ruminococcus, Coprococcus, Streptococcus, Dorea, Blautia, Ruminococcus, Lactobacillus, Enterococcus, Streptococcus, Actinomyces, Lactococcus, Roseburia, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroides, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Pediococcus, Citrobacter, Odoribacter, Enterobacteria, Fusobacterium, or Proteus, or two or more thereof. In some embodiments the target bacteria cells comprise Escherichia coli, Fusobacterium nucleatum, Haemophilus parainfluenzae (Pasteurellaceae), Veillonella parvula, Eikenella corrodens (Neisseriaceae), Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, or Coprococcus iomes, or two or more thereof. In some embodiments the lytic bacteriophage is a recombinant Tequatrovirus (e.g., p00ex), Slopekvirus (e.g., p1240), or Marfavirus (e.g., p⁵⁹18). In some embodiments the lytic bacteriophage is a recombinant Alcyoneusvirus, Asteriusvirus, Bifseptvirus, Biseptimavirus, Bonnellvirus, Chivirus, Dhakavirus, Dhillonvirus, Drulisvirus, Epseptimavirus, Felixounavirus, Gamaleyavirus, Gequatrovirus, Goslavirus, Guelphvirus, Hanrivervirus, Inovirus, Jiaodavirus, Kagunavirus, Kayfunavirus, Krischvirus, Kuravirus, Lederbergvirus, Levivirus, Mosigvirus, Nonagvirus, Peduovirus, Phapecoctavirus, Przondovirus, Rogunavirus, Saphexavirus, Schiekvirus, Seunavirus, Seuratvirus, Skarprettervirus, Slopekvirus, Sugarlandvirus, Taipeivirus, Tequatrovirus, Tequintavirus, Teseptimavirus, Uetakavirus, Vectrevirus, Vequintavirus, or Webervirus. In some embodiments the lytic bacteriophage is a recombinant P1 phage, M13 phage, λ phage, T4 phage, T7 phage, T7m phage, ϕC2 phage, ϕCD27 phage, ϕNM1 phage, Bc431 v3 phage, ϕ10 phage, ϕ25 phage, ϕ151 phage, A511-like phage, B054, 0176-like phage, Campylobacter phage, ϕCD146 C. difficile bacteriophage, or ϕCD24-2 C. difficile bacteriophage. In some embodiments the lytic bacteriophage comprises a nucleic acid encoding a lytic gene sequence. In some embodiments the lytic gene sequence regulates the production of holin. In some embodiments the lytic gene sequence comprises lysin. In some embodiments the nucleic acid encoding the therapeutic peptide is inserted in the Dell, late sigma transcription factor site, or r3 anti-holin site. In some embodiments the lytic bacteriophage comprises a promoter operably linked to the nucleic acid encoding the therapeutic peptide. In some embodiments the promoter is at least 80% or 90% identical to any one of SEQ ID NOS: 1-66 or 74-81. In some embodiments the promoter is L-arabinose inducible promoter, lac promoter, L-rhamnose inducible promoter, T7 RNA polymerase promoter, trc promoter, tac promoter, lambda phage promoter, anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoter, thr, horn, diphtheria toxin promoter, sig A, sig B, nusG, SoxS, katb, a-amylase (Pamy), Ptms, P43, Ptms, P43, rplK-rplA, ferredoxin promoter, xylose promoter, or BBa_J23102 promoter. In some embodiments the promoter is a bacterial promoter. In some embodiments the promoter is a phage promoter. In some embodiments the lytic bacteriophage does not incorporate into the host organism genome or the target bacteria genome. In some embodiments the therapeutic peptide comprises an anti-inflammatory peptide. In some embodiments the anti-inflammatory peptide comprises pancreatitis-associated protein, Mycobacterium leprae Hsp65, bioactive heme oxygenase 1, EDSGTT, or TNFRI peptide, or two or more thereof. In some embodiments the therapeutic peptide comprises a cytokine. In some embodiments the therapeutic peptide comprises an interleukin. In some embodiments the interleukin comprises IL-4, IL-6, IL-9, IL-10, IL-11, IL-13, IL-19, IL-27, IL-35, or IL-37, or two or more thereof. In some embodiments the therapeutic peptide comprises an antibody. In some embodiments the antibody is a nanobody. In some embodiments the antibody affects TNF-α signaling, IL-1 signaling, IL-6 signaling, IL-4 signaling, IL-13 signaling, IL-2 signaling, TGF-0 signaling, EGF signaling, HGH signaling, IGF signaling, NGF signaling, ROS1 signaling, ALK signaling, IFNγ signaling, IDO signaling, PD-1 signaling, PD-L1 signaling, CTLA-4 signaling, LAG-3 signaling, VISTA signaling, TIM-3 signaling, MMP signaling, VEGF signaling, or Wnt signaling, or two or more thereof. In some embodiments the antibody binds to a pro-inflammatory peptide. In some embodiments the antibody binds to TNF-α, IL12, IL-17, IL-17A, IL-17R, IL-23, IL-23A, MAdCAM-1, α4β7-integrin, α4β1-integrin, or αEβ7-integrin, or two or more thereof. In some embodiments the host organism has inflammation. In some embodiments a therapeutically effective amount of the therapeutic peptide is produced to treat the inflammation. In some embodiments the host organism has cancer. In some embodiments a therapeutically effective amount of the therapeutic peptide is produced to treat the cancer. In some embodiments the host organism has Inflammatory Bowel Disease, Ulcerative Colitis, Crohn's Disease, Rheumatoid Arthritis, Psoriasis, Psoriatic Arthritis, Systemic Lupus Erythematosus, Lupus Nephritis, or cancer. In some embodiments a therapeutically effective amount of the therapeutic peptide is produced to treat the Inflammatory Bowel Disease, Ulcerative Colitis, Crohn's Disease, Rheumatoid Arthritis, Psoriasis, Psoriatic Arthritis, Systemic Lupus Erythematosus, Lupus Nephritis, or cancer.

In certain aspects, disclosed herein is a combination comprising a lytic bacteriophage comprising a nucleic acid encoding a therapeutic peptide, and a plurality of target bacterial cells, wherein the therapeutic peptide is produced within one or more of the plurality of target bacterial cells in the combination. In some embodiments the bacteriophage and the plurality of target bacterial cells co-exist for at least about 1 day after contact. In some embodiments the therapeutic peptide is released from the combination for at least about 1 day. In some embodiments a therapeutically effective amount of the therapeutic peptide is produced without eliminating the plurality of the target bacterial cells. In some embodiments a therapeutically effective amount of the therapeutic peptide is produced, and the plurality of target bacterial cells remains above the limit of detection for at least about 1 day after contact. In some embodiments detection is based on presence of colonies after plating on an agar plate. In some embodiments the target bacterial cells comprise E. coli, K. pneumoniae, R. gnavus, E. gallinarum, E. faecalis, E. faecium, or B. fragilis, or two or more thereof. In some embodiments the target bacterial cells comprise Enterobacteriaceae, Pasteurellaceae, Fusobacteriaceae, Neisseriaceae, Veillonellaceae, Gemellaceae, Bacteriodales, Clostridiales, Erysipelotrichaceae, Bifidobacteriaceae, Bacteroides, Faecalibacterium, Roseburia, Blautia, Ruminococcus, Coprococcus, Streptococcus, Dorea, Blautia, Ruminococcus, Lactobacillus, Enterococcus, Streptococcus, Actinomyces, Lactococcus, Roseburia, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroides, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Pediococcus, Citrobacter, Odoribacter, Enterobacteria, Fusobacterium, or Proteus, or two or more thereof. In some embodiments the target bacteria cells comprise Escherichia coli, Fusobacterium nucleatum, Haemophilus parainfluenzae (Pasteurellaceae), Veillonella parvula, Eikenella corrodens (Neisseriaceae), Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, or Coprococcus iomes, or two or more thereof. In some embodiments the lytic bacteriophage is a recombinant Tequatrovirus (e.g., p00ex), Slopekvirus (e.g., p1240), or Marfavirus (e.g., p⁵⁹18). In some embodiments the lytic bacteriophage is a recombinant Alcyoneusvirus, Asteriusvirus, Bifseptvirus, Biseptimavirus, Bonnellvirus, Chivirus, Dhakavirus, Dhillonvirus, Drulisvirus, Epseptimavirus, Felixounavirus, Gamaleyavirus, Gequatrovirus, Goslavirus, Guelphvirus, Hanrivervirus, Inovirus, Jiaodavirus, Kagunavirus, Kayfunavirus, Krischvirus, Kuravirus, Lederbergvirus, Levivirus, Mosigvirus, Nonagvirus, Peduovirus, Phapecoctavirus, Przondovirus, Rogunavirus, Saphexavirus, Schiekvirus, Seunavirus, Seuratvirus, Skarprettervirus, Slopekvirus, Sugarlandvirus, Taipeivirus, Tequatrovirus, Tequintavirus, Teseptimavirus, Uetakavirus, Vectrevirus, Vequintavirus, or Webervirus. In some embodiments the lytic bacteriophage is a recombinant P1 phage, M13 phage, λ phage, T4 phage, T7 phage, T7m phage, ϕC2 phage, ϕCD27 phage, ϕNM1 phage, Bc431 v3 phage, ϕ10 phage, ϕ25 phage, ϕ151 phage, A511-like phage, B054, 0176-like phage, Campylobacter phage, ϕCD146 C. difficile bacteriophage, or ϕCD24-2 C. difficile bacteriophage. In some embodiments the lytic bacteriophage comprises a nucleic acid encoding a lytic gene sequence. In some embodiments the lytic gene sequence regulates the production of holin. In some embodiments the lytic gene sequence comprises lysin. In some embodiments the nucleic acid encoding the therapeutic peptide is inserted in the Dell, late sigma transcription factor site, or r3 anti-holin site. In some embodiments the lytic bacteriophage comprises a promoter operably linked to the nucleic acid encoding the therapeutic peptide. In some embodiments the promoter is at least 80% or 90% identical to any one of SEQ ID NOS: 1-66 or 74-81. In some embodiments the promoter is L-arabinose inducible promoter, lac promoter, L-rhamnose inducible promoter, T7 RNA polymerase promoter, trc promoter, tac promoter, lambda phage promoter, anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoter, thr, horn, diphtheria toxin promoter, sig A, sig B, nusG, SoxS, katb, a-amylase (Pamy), Ptms, P43, Ptms, P43, rplK-rplA, ferredoxin promoter, xylose promoter, or BBa_J23102 promoter. In some embodiments the promoter is a bacterial promoter. In some embodiments the promoter is a phage promoter. In some embodiments the lytic bacteriophage does not incorporate into the target bacteria genome. In some embodiments the therapeutic peptide comprises an anti-inflammatory peptide. In some embodiments the anti-inflammatory peptide comprises pancreatitis-associated protein, Mycobacterium leprae Hsp65, bioactive heme oxygenase 1, EDSGTT, or TNFRI peptide, or two or more thereof. In some embodiments the therapeutic peptide comprises a cytokine. In some embodiments the therapeutic peptide comprises an interleukin. In some embodiments the interleukin comprises IL-4, IL-6, IL-9, IL-10, IL-11, IL-13, IL-19, IL-27, IL-35, or IL-37, or two or more thereof. In some embodiments the therapeutic peptide comprises an antibody. In some embodiments the antibody is a nanobody. In some embodiments the antibody affects TNF-α signaling, IL-1 signaling, IL-6 signaling, IL-4 signaling, IL-13 signaling, IL-2 signaling, TGF-β signaling, EGF signaling, HGH signaling, IGF signaling, NGF signaling, ROS1 signaling, ALK signaling, IFNγ signaling, IDO signaling, PD-1 signaling, PD-L1 signaling, CTLA-4 signaling, LAG-3 signaling, VISTA signaling, TIM-3 signaling, MMP signaling, VEGF signaling, or Wnt signaling, or two or more thereof. In some embodiments the antibody binds to a pro-inflammatory peptide. In some embodiments the antibody binds to TNF-α, IL12, IL-17, IL-17A, IL-17R, IL-23, IL-23A, MAdCAM-1, α4β7-integrin, α4β1-integrin, or αEβ7-integrin, or two or more thereof. In some embodiments a therapeutically effective amount of the therapeutic peptide is produced to treat the inflammation. In some embodiments a therapeutically effective amount of the therapeutic peptide is produced to treat the cancer. In some embodiments a therapeutically effective amount of the therapeutic peptide is produced to treat the Inflammatory Bowel Disease, Ulcerative Colitis, Crohn's Disease, Rheumatoid Arthritis, Psoriasis, Psoriatic Arthritis, Systemic Lupus Erythematosus, Lupus Nephritis, or cancer.

In certain aspects, disclosed herein is a lytic bacteriophage comprising a nucleic acid encoding a therapeutic peptide. In some embodiments the lytic bacteriophage targets E. coli, K. pneumoniae, R. gnavus, E. gallinarum, E. faecalis, E. faecium, or B. fragilis, or two or more thereof. In some embodiments the lytic phage targets Enterobacteriaceae, Pasteurellaceae, Fusobacteriaceae, Neisseriaceae, Veillonellaceae, Gemellaceae, Bacteriodales, Clostridiales, Erysipelotrichaceae, Bifidobacteriaceae, Bacteroides, Faecalibacterium, Roseburia, Blautia, Ruminococcus, Coprococcus, Streptococcus, Dorea, Blautia, Ruminococcus, Lactobacillus, Enterococcus, Streptococcus, Actinomyces, Lactococcus, Roseburia, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroides, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Pediococcus, Citrobacter, Odoribacter, Enterobacteria, Fusobacterium, or Proteus, or two or more thereof. In some embodiments the lytic phage targets Escherichia coli, Fusobacterium nucleatum, Haemophilus parainfluenzae (Pasteurellaceae), Veillonella parvula, Eikenella corrodens (Neisseriaceae), Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, or Coprococcus iomes, or two or more thereof. In some embodiments the lytic bacteriophage is a recombinant Tequatrovirus (e.g., p00ex), Slopekvirus (e.g., p1240), or Marfavirus (e.g., p5918). In some embodiments the lytic bacteriophage is a recombinant Alcyoneusvirus, Asteriusvirus, Bifseptvirus, Biseptimavirus, Bonnellvirus, Chivirus, Dhakavirus, Dhillonvirus, Drulisvirus, Epseptimavirus, Felixounavirus, Gamaleyavirus, Gequatrovirus, Goslavirus, Guelphvirus, Hanrivervirus, Inovirus, Jiaodavirus, Kagunavirus, Kayfunavirus, Krischvirus, Kuravirus, Lederbergvirus, Levivirus, Mosigvirus, Nonagvirus, Peduovirus, Phapecoctavirus, Przondovirus, Rogunavirus, Saphexavirus, Schiekvirus, Seunavirus, Seuratvirus, Skarprettervirus, Slopekvirus, Sugarlandvirus, Taipeivirus, Tequatrovirus, Tequintavirus, Teseptimavirus, Uetakavirus, Vectrevirus, Vequintavirus, or Webervirus. In some embodiments the lytic bacteriophage is a recombinant P1 phage, M13 phage, λ phage, T4 phage, T7 phage, T7m phage, ϕC2 phage, ϕCD27 phage, ϕNM1 phage, Bc431 v3 phage, 4 ϕ10 phage, ϕ25 phage, ϕ151 phage, A511-like phage, B054, 0176-like phage, Campylobacter phage, ϕCD146 C. difficile bacteriophage, or ϕCD24-2 C. difficile bacteriophage. In some embodiments the lytic bacteriophage comprises a nucleic acid encoding a lytic gene sequence. In some embodiments the lytic gene sequence regulates the production of holin. In some embodiments the lytic gene sequence comprises lysin. In some embodiments the nucleic acid encoding the therapeutic peptide is inserted in the Dell, late sigma transcription factor site, or r3 anti-holin site of the phage. In some embodiments the lytic bacteriophage comprises a promoter operably linked to the nucleic acid encoding the therapeutic peptide. In some embodiments the promoter is at least 80% or 90% identical to any one of SEQ ID NOS: 1-66 or 74-81. In some embodiments the promoter is L-arabinose inducible promoter, lac promoter, L-rhamnose inducible promoter, T7 RNA polymerase promoter, trc promoter, tac promoter, lambda phage promoter, anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoter, thr, horn, diphtheria toxin promoter, sig A, sig B, nusG, SoxS, katb, a-amylase (Pamy), Ptms, P43, Ptms, P43, rplK-rplA, ferredoxin promoter, xylose promoter, or BBa_J23102 promoter. In some embodiments the promoter is a bacterial promoter. In some embodiments the promoter is a phage promoter. In some embodiments the lytic bacteriophage does not incorporate into its target bacteria genome. In some embodiments the therapeutic peptide comprises an anti-inflammatory peptide. In some embodiments the anti-inflammatory peptide comprises pancreatitis-associated protein, Mycobacterium leprae Hsp65, bioactive heme oxygenase 1, EDSGTT, or TNFRI peptide, or two or more thereof. In some embodiments the therapeutic peptide comprises a cytokine. In some embodiments the therapeutic peptide comprises an interleukin. In some embodiments the interleukin comprises IL-4, IL-6, IL-9, IL-10, IL-11, IL-13, IL-19, IL-27, IL-35, or IL-37, or two or more thereof. In some embodiments the therapeutic peptide comprises an antibody. In some embodiments the antibody is a nanobody. In some embodiments the antibody affects TNF-α signaling, IL-1 signaling, IL-6 signaling, IL-4 signaling, IL-13 signaling, IL-2 signaling, TGF-β signaling, EGF signaling, HGH signaling, IGF signaling, NGF signaling, ROS1 signaling, ALK signaling, IFNγ signaling, IDO signaling, PD-1 signaling, PD-L1 signaling, CTLA-4 signaling, LAG-3 signaling, VISTA signaling, TIM-3 signaling, MMP signaling, VEGF signaling, or Wnt signaling, or two or more thereof. In some embodiments the antibody binds to a pro-inflammatory peptide. In some embodiments the antibody binds to TNF-α, IL12, IL-17, IL-17A, IL-17R, IL-23, IL-23A, MAdCAM-1, α4β7-integrin, α4β1-integrin, or αEβ7-integrin, or two or more thereof. In some embodiments, disclosed herein is a method of treating a subject having inflammation, the method comprising administering to the subject the bacteriophage disclosed herein. In some embodiments a therapeutically effective amount of the therapeutic peptide is produced to treat the inflammation. A method of treating a subject having cancer, the method comprising administering to the subject the bacteriophage of any one of claims 74-100. In some embodiments a therapeutically effective amount of the therapeutic peptide is produced to treat the cancer. In some embodiments, disclosed herein is a method of treating a subject having Inflammatory Bowel Disease, Ulcerative Colitis, Crohn's Disease, Rheumatoid Arthritis, Psoriasis, Psoriatic Arthritis, Systemic Lupus Erythematosus, Lupus Nephritis, or cancer, the method comprising administering to the subject the bacteriophage disclosed herein. In some embodiments a therapeutically effective amount of the therapeutic peptide is produced to treat the Inflammatory Bowel Disease, Ulcerative Colitis, Crohn's Disease, Rheumatoid Arthritis, Psoriasis, Psoriatic Arthritis, Systemic Lupus Erythematosus, Lupus Nephritis, or cancer. In some embodiments the therapeutic peptide is soluble and is not expressed on a capsid protein of the bacteriophage. In some embodiments the therapeutic peptide is not a nuclease. In some embodiments an operable lytic gene sequence regulates the lytic cycle of the bacteriophage.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosures will be obtained by reference to the following detailed description that sets forth illustrative embodiments, in which the principles of the disclosures are utilized, and the accompanying drawings of which:

FIG. 1 depicts a schematic of non-engineered wild type (wt) phage genome as compared to engineered variants carrying the IL-10 interleukin construct (SEQ ID NO: 67, encoding SEQ ID NO: 68) within the p00EX deleted regions of the phage genome.

FIG. 2 depicts a schematic of non-engineered wild type (wt) phage genome as compared to engineered variants carrying the Tumor necrosis factor alpha (TNFα) nanobody construct within the p00EX deleted regions of the phage genome.

FIG. 3 depicts a schematic of non-engineered wild type (wt) phage genome as compared to engineered variants carrying the TNFα Receptor peptide (TNFRI) peptide construct into the p00EX region of the phage genome.

FIG. 4 shows optimization of expression magnitude and timing from phage genomes yields tunable delivery of a recombinant protein during phage amplification, as further described in Example 22. Briefly, the first panel shows that payload insertion can sometimes result in low protein expression, the second panel shows that engineering optimization allows maximization of protein expression, and the third panel shows high-throughput screening identifies optimized variants with a range of expression levels.

FIG. 5 shows that various promoters have been identified that successfully express proteins from multiple sites in a phage, as further described in Example 23. Briefly, the first panel shows that low-throughput testing showed that insertion site and promoter have combinatorial effects on construct expression, the second panel shows that higher throughput testing across numerous sites still shows combinatorial effects, but with some promoters trending stronger than others across sites, and the third panel shows that high throughput testing of numerous promoters shows that largely site-agnostic promoters can be identified. For the second panel, the columns are labeled from left to right as follows: p1240 MajorCap, p1240 pDNAPol, p1240 pHolin, p1240 pTopois, p5918 pMajHead, p5918 pRecA, p5918 ProHead, and pBba J23109; and the rows are labeled from top to bottom as follows: p1240 ins10, p1240 ins7, p5918 ins10, p5918 ins12, p5918 ins2, p5918 ins4, p5918 ins5, and p5918 ins9.

FIG. 6 shows that phage-expressed payloads are detectable in vivo using a murine model of GI inflammation, validating the opportunity to deliver biotherapeutic proteins to inflamed areas of the gut, as further described in Example 24.

FIG. 7 shows that phages express immune modifiers that are active against their biological target when released from phage-infected cells, as further described in Example 25.

FIG. 8 depicts viability of E. coli after treatment with Humira or a phage-derived anti-TNFα nanobody (5M2M, 5M2I).

FIG. 9 depicts the population levels of bacteriophage and bacteria in a subject over time.

### DETAILED DESCRIPTION

### Compositions of Matter

Disclosed herein in certain aspects, is a bacteriophage configured to infect a bacterial cell, wherein the bacteriophage comprises a nucleic acid comprising a first sequence encoding an therapeutic peptide and a second nucleic acid sequence encoding an operable lytic gene capable of inducing lysis of a target bacterium during a lytic cycle of the recombinant lytic bacteriophage. In certain aspects, the bacteriophage is replicative. In certain embodiments, the bacteriophage is replication incompetent. In certain aspects, the therapeutic peptide comprises an anti-inflammatory peptide. In certain aspects, the therapeutic peptide comprises an interleukin. In certain aspects, the therapeutic peptide comprises an antibody.

### Interleukins

Interleukins are cytokines involved in immune response regulation. They are generally synthesized by immune cells in response to pathogens or other danger signals capable of upregulating immune responses. Following synthesis and secretion, interleukins are capable of interacting with and modulating neighboring cells. Interleukins are capable of diverse functions, which may vary based on the specific interleukin, the targeted binding cell, and cellular signaling variations. Interleukins alter cellular gene expression to either increase or decrease inflammation. Secreted interleukins often bind to a receptor on the target cell surface. Interleukin binding to the target cell receptor can initiate a signaling cascade that alters host cell transcription. In specific, interleukin 10 (IL-10) dampens several classically pro-inflammatory cytokines like IFN-7, IL-2, and TNF-α. Autoimmunity or dysregulation of the inflammatory response can be addressed by proper anti-inflammatory interleukin signaling. In certain embodiments, the bacteriophage described herein comprise a nucleic acid sequence comprising a first sequence encoding an interleukin.

In some embodiments, the bacteriophage described herein comprises a nucleic acid encoding an interleukin. In some embodiments, the interleukin is an anti-inflammatory interleukin. In some embodiments, the anti-inflammatory interleukin is an IL-4, IL-6, IL-9, IL-10, IL-11, IL-13, IL-19, IL-27, IL-35 and IL-37. In some embodiments, the anti-inflammatory interleukin is an IL-4. In some embodiments, the anti-inflammatory interleukin is an IL-6. In some embodiments, the anti-inflammatory interleukin is an IL-9. In some embodiments, the anti-inflammatory interleukin is an IL-10. In some embodiments, the anti-inflammatory interleukin is an IL-11. In some embodiments, the anti-inflammatory interleukin is an IL-13. In some embodiments, the anti-inflammatory interleukin is an IL-19. In some embodiments, the anti-inflammatory interleukin is an IL-27. In some embodiments, the anti-inflammatory interleukin is an IL-35. In some embodiments, the anti-inflammatory interleukin is an IL-37.

In some embodiments, the anti-inflammatory interleukin is exogenous to the target bacterium. In some embodiments, the anti-inflammatory interleukin is exogenous to the bacteriophage. In some embodiments, the anti-inflammatory interleukin is freely soluble when expressed. In some embodiments, the synthesized anti-inflammatory interleukin is not fused to phage structural genes. In some embodiments, the bacteriophage comprises a sequence encoding an anti-inflammatory interleukin, wherein the bacteriophage does not comprise the anti-inflammatory interleukin. In some embodiments, the capsid of the bacteriophage does not comprise an anti-inflammatory interleukin.

### Antibodies

Antibodies are immune-derived proteins that are designed to recognize and bind to specific antigens. They are typically synthesized in response to an antigen being presented to a B lymphocyte, triggering it to divide and transform into a plasma cell whereby it produces millions of antibodies. Antibodies, however, have been produced synthetically through recombinant methods (e.g., introduction of recombinant DNA into a mammalian cell expression system) and used to treat numerous diseases from cancer (e.g., rituximab in non-Hodgkin's lymphoma) to inflammatory disorders (e.g., adalimumab in rheumatoid arthritis). The specificity of antibodies is useful when targeting signaling molecules and/or receptors, to which antibodies can bind and prevent signaling from occurring. Tumor necrosis factor alpha (TNFα) is a pro-inflammatory cytokine that is involved in several inflammation-associated diseases (e.g., inflammatory bowel disease, rheumatoid arthritis, plaque psoriasis, ankylosing spondylitis), for which multiple anti-TNFα antibodies have been developed (e.g., adalimumab, infliximab, certolizumab pegol, and golimumab). In certain embodiments, the bacteriophage described herein comprise a nucleic acid sequence comprising a first sequence encoding an antibody targeting TNFα.

In some embodiments, the bacteriophage described herein comprises a nucleic acid encoding an antibody. In some embodiments, the bacteriophage described herein comprises a nucleic acid encoding a conventional antibody that consists of a heavy & light chain. In some embodiments, the bacteriophage described herein comprises a nucleic acid encoding a heavy chain antibody. In some embodiments, the bacteriophage described herein comprises a nucleic acid encoding an Fc fusion protein. In some embodiments, the bacteriophage described herein comprises a nucleic acid encoding a bispecific antibody. In some embodiments, the bacteriophage described herein comprises a nucleic acid encoding an antigen binding fragment (Fab). In some embodiments, the bacteriophage described herein comprises a nucleic acid encoding a single chain variable fragment (ScFv). In some embodiments, the bacteriophage described herein comprises a nucleic acid encoding a single-domain antibody fragment (i.e., nanobody).

In some embodiments, the antibody targets a pro-inflammatory peptide. In some embodiments, the antibody targets a pro-inflammatory protein. In some embodiments, the antibody targets a pro-inflammatory cytokine. In some embodiments, the antibody targets a cytokine that have pro- and anti-inflammatory activity. In some embodiments, the antibody targets a pro-inflammatory interleukin. In some embodiments, the antibody targets an interleukin that have pro- and anti-inflammatory activity. In some embodiments, the antibody targets a cytokine receptor. In some embodiments, the antibody targets an interleukin receptor. In some embodiments, the antibody targets an integrin receptor. In some embodiments, the antibody targets an adhesion molecule. In some embodiments, the antibody targets TNF-α. In some embodiments, the antibody targets IL12. In some embodiments, the antibody targets IL-17. In some embodiments, the antibody targets IL-17A. In some embodiments, the antibody targets IL-17R. In some embodiments, the antibody targets IL-23. In some embodiments, the antibody targets IL-23A. In some embodiments, the antibody targets MAdCAM-1. In some embodiments, the antibody targets α4β7-integrin. In some embodiments, the antibody targets α4β1-integrin. In some embodiments, the antibody targets αEβ7-integrin.

In some embodiments, the antibody is exogenous to the target bacterium. In some embodiments, the antibody is exogenous to the bacteriophage. In some embodiments, the antibody is freely soluble when expressed. In some embodiments, the bacteriophage comprises a sequence encoding an antibody, wherein the bacteriophage does not comprise the antibody. In some embodiments, the capsid of the bacteriophage does not comprise an antibody.

### Anti-Inflammatory Proteins & Peptides

Several peptides and proteins have been shown to bind to receptors on eukaryotic cells and modulate inflammation either through activation or inhibition.

In some embodiments, the bacteriophage described herein comprises a nucleic acid encoding an anti-inflammatory peptide. In some embodiments, the anti-inflammatory peptide is pancreatitis-associated protein. In some embodiments, the anti-inflammatory peptide is Mycobacterium leprae Hsp65. In some embodiments, the anti-inflammatory peptide is bioactive heme oxygenase 1 (mHO-1). In some embodiments, the anti-inflammatory peptide is EDSGTT peptide. In some embodiments, the anti-inflammatory peptide a TNFα Receptor peptide (TNFRI).

In some embodiments, the anti-inflammatory peptide is exogenous to the target bacterium. In some embodiments, the anti-inflammatory peptide is exogenous to the bacteriophage. In some embodiments, the anti-inflammatory peptide is freely soluble when expressed. In some embodiments, the bacteriophage comprises a sequence encoding a anti-inflammatory peptide, wherein the bacteriophage does not comprise the anti-inflammatory peptide. In some embodiments, the capsid of the bacteriophage does not comprise a anti-inflammatory peptide.

### Phages

Disclosed herein, in certain embodiments, are bacteriophage compositions and methods of use thereof.

Bacteriophages or “phages” represent a group of bacterial viruses and are engineered or sourced from environmental sources. Individual bacteriophage host ranges are usually narrow, meaning phages are highly specific to one strain or few strains of a bacterial species and this specificity makes them unique in their antibacterial action. Bacteriophages are bacterial viruses that rely on the host's cellular machinery to replicate. Bacteriophages are generally classified as virulent or temperate phages depending on their lifestyle. Virulent bacteriophages, also known as lytic bacteriophages, can only undergo lytic replication. Lytic bacteriophages infect a host cell, undergo numerous rounds of replication, and trigger cell lysis to release newly made bacteriophage particles. In some embodiments, the lytic bacteriophages disclosed herein retain their replicative ability. In some embodiments, the lytic bacteriophages disclosed herein retain their ability to trigger cell lysis. In some embodiments, the lytic bacteriophages disclosed herein retain both their replicative ability and the ability to trigger cell lysis. In some embodiments, the bacteriophages disclosed herein encode a therapeutic peptide. In some embodiments, the encoded therapeutic peptide does not affect the bacteriophage's ability to replicate and/or trigger cell lysis. Temperate or lysogenic bacteriophages can undergo lysogeny in which the phage stops replicating and stably resides within the host cell, either integrating into the bacterial genome or being maintained as an extrachromosomal plasmid. Temperate phages can also undergo lytic replication similar to their lytic bacteriophage counterparts. Whether a temperate phage replicates lytically or undergoes lysogeny upon infection depends on a variety of factors including growth conditions and the physiological state of the cell. A bacterial cell that has a lysogenic phage integrated into its genome is referred to as a lysogenic bacterium or lysogen. Exposure to adverse conditions may trigger reactivation of the lysogenic phage, termination of the lysogenic state and resumption of lytic replication by the phage. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication. In some embodiments, the temperate bacteriophages disclosed herein are rendered lytic. The term “lysogeny gene” refers to any gene whose gene product promotes lysogeny of a temperate phage. Lysogeny genes can directly promote, as in the case of integrase proteins, integration of the bacteriophage into the host genome. Lysogeny genes can also indirectly promote lysogeny as in the case of CI transcriptional regulators which prevent transcription of genes required for lytic replication and thus favor maintenance of lysogeny.

Bacteriophages package and deliver synthetic DNA using three general approaches. Under the first approach, the synthetic DNA is recombined into the bacteriophage genome in a targeted manner, which can involve the use of a selection or counterselection marker. Under the second approach, restriction sites within the phage are used to introduce synthetic DNA in-vitro. Under the third approach, a plasmid generally encoding the phage packaging sites and lytic origin of replication is packaged as part of the assembly of the bacteriophage particle. The resulting plasmids have been coined “phagemids.”

Phages are limited to a given bacterial strain for evolutionary reasons. In some cases, injecting their genetic material into an incompatible strain is counterproductive. Phages have therefore evolved to specifically infect a limited cross-section of bacterial strains. However, some phages have been discovered that inject their genetic material into a wide range of bacteria. The classic example is the P1 phage, which has been shown to inject DNA in a range of gram-negative bacteria.

Disclosed herein, in some embodiments, are bacteriophages comprising a first nucleic acid sequence encoding an therapeutic peptide, or the RNA transcribed therefrom, wherein the bacteriophage does not comprise the therapeutic peptide. In some embodiments, the bacteriophage comprises a first nucleic acid sequence encoding an therapeutic peptide, or the RNA transcribed therefrom, wherein the bacteriophage does not comprise the therapeutic peptide, provided that the bacteriophage is rendered lytic. In some embodiments, the bacteriophage comprises a second nucleic acid sequence encoding an operable lytic gene capable of inducing lysis of a target bacterium. In some embodiments, the bacteriophage comprises a second nucleic acid sequence encoding an operable lytic gene capable of inducing lysis of a target bacterium during a lytic cycle of the recombinant lytic bacteriophage. In some embodiments, the bacteriophage is a temperate bacteriophage. In some embodiments, the bacteriophage is a recombinant lytic bacteriophage. In some embodiments, the bacteriophage is a replication competent recombinant lytic bacteriophage.

In some embodiments, the bacteriophage is rendered lytic by removal, replacement, or inactivation of a lysogenic gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the bacteriophage is rendered lytic by the removal of a regulatory element of a lysogeny gene. In some embodiments, the bacteriophage is rendered lytic by the removal of a promoter of a lysogeny gene. In some embodiments, the bacteriophage is rendered lytic by the removal of a functional element of a lysogeny gene. In some embodiments, the lysogenic gene is an activator gene. In some embodiments, the lysogenic gene is cII gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, the bacteriophage is rendered lytic by the insertion of one or more lytic genes. In some embodiments, the bacteriophage is rendered lytic by the insertion of one or more genes that contribute to the induction of a lytic cycle. In some embodiments, the bacteriophage is rendered lytic by altering the expression of one or more genes that contribute to the induction of a lytic cycle. In some embodiments, the bacteriophage phenotypically changes from a lysogenic bacteriophage to a lytic bacteriophage. In some embodiments, the bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI)). In some embodiments, the bacteriophage that is rendered lytic is prevented from reverting to lysogenic state. In some embodiments, the bacteriophage that is rendered lytic is prevented from reverting back to lysogenic state by way of introducing an additional nucleic acid sequence. In some embodiments, the bacteriophage does not confer any new properties onto the target bacterium beyond cellular death caused by lytic activity of the bacteriophage and/or the activity of the additional nucleic acid sequence. Further disclosed herein, in some embodiments, are temperate bacteriophages comprising a first nucleic acid sequence encoding an therapeutic peptide or an RNA transcribed therefrom, wherein the bacteriophage does not comprise the therapeutic peptide. In some embodiments, the bacteriophage infects multiple bacterial strains.

In some embodiments, the bacteriophage or phagemid DNA is from a lysogenic or temperate bacteriophage. In some embodiments, the bacteriophages or phagemids include but are not limited to Alcyoneusvirus, Asteriusvirus, Bifseptvirus, Biseptimavirus, Bonnellvirus, Chivirus, Dhakavirus, Dhillonvirus, Drulisvirus, Epseptimavirus, Felixounavirus, Gamaleyavirus, Gequatrovirus, Goslavirus, Guelphvirus, Hanrivervirus, Inovirus, Jiaodavirus, Kagunavirus, Kayfunavirus, Krischvirus, Kuravirus, Lederbergvirus, Levivirus, Mosigvirus, Nonagvirus, Peduovirus, Phapecoctavirus, Przondovirus, Rogunavirus, Saphexavirus, Schiekvirus, Seunavirus, Seuratvirus, Skarprettervirus, Slopekvirus, Sugarlandvirus, Taipeivirus, Tequatrovirus, Tequintavirus, Teseptimavirus, Uetakavirus, Vectrevirus, Vequintavirus, Webervirus, P1 phage, a M13 phage, a λ phage, a T4 phage, a T7 phage, a T7m phage, a ϕC2 phage, a ϕCD27 phage, a ϕNM1 phage, Bc431 v3 phage, 410 phage, ϕ25 phage, ϕ151 phage, A511-like phages, B054, 0176-like phages, or Campylobacter phages (such as NCTC 12676 and NCTC 12677). In some embodiments, the bacteriophage is ϕCD146 C. difficile bacteriophage. In some embodiments, the bacteriophage is ϕCD24-2 C. difficile bacteriophage.

In some embodiments, a plurality of bacteriophages are used together. In some embodiments, the plurality of bacteriophages used together targets the same or different bacteria within a sample or subject. In some embodiments, the bacteriophages used together comprises T4 phage, T7 phage, T7m phage, or any combination of bacteriophages described herein.

In some embodiments, bacteriophages of interest are obtained from environmental sources or commercial research vendors. In some embodiments, obtained bacteriophages are screened for lytic activity against a library of bacteria. In some embodiments, the bacteriophages are screened against a library of bacteria for their ability to generate primary resistance in the screened bacteria.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

In some embodiments, the bacteriophage is ϕCD146 C. difficile bacteriophage. In some embodiments, the bacteriophage is ϕCD24-2 C. difficile bacteriophage.

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is gp49 from ϕCD146 C. difficile bacteriophage. In some embodiments, the non-essential gene to be removed and/or replaced from the bacteriophage is gp75 from ϕCD24-2 C. difficile bacteriophage.

Disclosed herein, in certain embodiments, are bacteriophages comprising a nucleic acid encoding an therapeutic peptide. In some embodiments, the therapeutic peptide is an interleukin. In some embodiments, the interleukin is an anti-inflammatory interleukin. In some embodiments, the anti-inflammatory interleukin comprises IL-4, IL-6, IL-9, IL-11, IL-13, IL-19, IL-35 and IL-37. In some embodiments, the anti-inflammatory interleukin comprises IL-10. In some embodiments, the therapeutic peptide encodes an antibody. In some embodiments, the antibody targets a pro-inflammatory peptide. In some embodiments, the antibody targets a pro-inflammatory protein. In some embodiments, the antibody targets a pro-inflammatory cytokine. In some embodiments, the therapeutic peptide comprises an anti-inflammatory peptide. In some embodiments, the anti-inflammatory peptide comprises pancreatitis-associated protein, Mycobacterium leprae Hsp65, bioactive heme oxygenase 1, EDSGTT, or TNFα Receptor peptide (TNFRI) peptide.

### Regulatory Elements

In some embodiments, the nucleic acid sequences are operatively associated with a variety of promoters, terminators and other regulatory elements for expression in various organisms or cells. In some embodiments, the nucleic acid sequence further comprises a leader sequence. In some embodiments, the nucleic acid sequence further comprises a promoter sequence. In some embodiments, at least one promoter and/or terminator is operably linked to the sequence encoding the therapeutic peptide. Any promoter useful with this disclosure is used and includes, for example, promoters functional with the organism of interest as well as constitutive, inducible, developmentally regulated, tissue-specific/preferred-promoters, and the like, as disclosed herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of the nucleic acid sequence is constitutive, inducible, temporally regulated, developmentally regulated, or chemically regulated. In some embodiments, the expression of the nucleic acid sequence is made constitutive, inducible, temporally regulated, developmentally regulated, or chemically regulated by operatively linking the nucleic acid sequence to a promoter functional in an organism of interest. In some embodiments, repression is made reversible by operatively linking the nucleic acid sequence to an inducible promoter that is functional in an organism of interest. The choice of promoter disclosed herein varies depending on the quantitative, temporal and spatial requirements for expression, and also depending on the host cell to be transformed.

Exemplary promoters for use with the methods, bacteriophages and compositions disclosed herein include promoters that are functional in bacteria. For example, L-arabinose inducible (araBAD, PBAD) promoter, any lac promoter, L-rhamnose inducible (rhaPBAD) promoter, T7 RNA polymerase promoter, trc promoter, tac promoter, lambda phage promoter (pLpL-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, a-amylase (Pamy), Ptms, P43 (comprised of two overlapping RNA polymerase a factor recognition sites, aA, aB), Ptms, P43, rplK-rplA, ferredoxin promoter, and/or xylose promoter. In some embodiments, the promoter is a BBa_J23102 promoter. In some embodiments, the promoter works in a broad range of bacteria, such as BBa_J23104, BBa_J23109. In some embodiments the promoter is derived from the target bacterium, such as p16, plpp, or ptat. In some embodiments, the promoter is a phage promoter, such as the promoter for P. aeruginosa phage phiKZ gene gp105 or gene gp245.

In some embodiments, the promoter comprises at least or about 70%, 80%, 85%, 90%, 91%, 92%, 93%, 94%, 95%, 96%, 97%, 98%, 99%, or 100% sequence identity to any one of SEQ ID NOs: 1-66 or 74-81. In some instances, the promoter comprises at least or about 95% homology to any one of SEQ ID NOs: 1-66 or 74-81. In some instances, the promoter comprises at least or about 97% homology to any one of SEQ ID NOs: 1-66 or 74-81. In some instances, the promoter comprises at least or about 99% homology to any one of SEQ ID NOs: 1-66 or 74-81. In some instances, the promoter comprises 100% homology to any one of SEQ ID NOs: 1-66 or 74-81. In some instances, the promoter comprises at least a portion having at least or about 3, 4, 5, 6, 7, 8, 9, 10, 12, 14, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more than 50 nucleotides of any one of SEQ ID NOs: 1-66 or 74-81. In some instances, the promoter comprises at least a portion having at least or about 50, 55, 60, 65, 70, 75, 80, 85, 90, 95, 100, 105, 110, 115 120, 125, 130, 135, 140, 145, 150, 155, 160, 165, 170, 175, 180, 185, 190, 195, 200, 205, 210, 215, or more than 215 nucleotides of any one of SEQ ID NOs: 1-66 or 74-81.

In some embodiments, inducible promoters are used. In some embodiments, chemical-regulated promoters are used to modulate the expression of a gene in an organism through the application of an exogenous chemical regulator. The use of chemically regulated promoters enables RNAs and/or the polypeptides encoded by the nucleic acid sequence to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

### Expression Cassette

In some embodiments, the nucleic acid sequence is an expression cassette or in an expression cassette. In some embodiments, the expression cassettes are designed to express the nucleic acid sequence disclosed herein. In some embodiments, the nucleic acid sequence is an expression cassette encoding an therapeutic peptide. In some embodiments, the therapeutic peptide comprises an anti-inflammatory peptide. In some embodiments, the therapeutic peptide comprises an interleukin. In some embodiments, the therapeutic peptide comprises an antibody.

In some embodiments, an expression cassette comprising a nucleic acid sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleic acid sequence of interest and for correct mRNA polyadenylation. In some embodiments, the termination region is native to the transcriptional initiation region, is native to the operably linked nucleic acid sequence of interest, is native to the host cell, or is derived from another source (i.e., foreign or heterologous to the promoter, to the nucleic acid sequence of interest, to the host, or any combination thereof). In some embodiments, terminators are operably linked to the nucleic acid sequence disclosed herein.

In some embodiments, an expression cassette includes a nucleotide sequence for a selectable marker. In some embodiments, the nucleotide sequence encodes either a selectable or a screenable marker, depending on whether the marker confers a trait that is selected for by chemical means, such as by using a selective agent (e.g. an antibiotic), or on whether the marker is simply a trait that one identifies through observation or testing, such as by screening (e.g., fluorescence).

### Vectors

In addition to expression cassettes, the nucleic acid sequences disclosed herein (e.g. nucleic acid sequence comprising an therapeutic peptide) are used in connection with vectors. A vector comprises a nucleic acid molecule comprising the nucleotide sequence(s) to be transferred, delivered or introduced. Non-limiting examples of general classes of vectors include, but are not limited to, a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector transforms a prokaryotic or eukaryotic host either by integration into the cellular genome or by existing extrachromosomally (e.g. as an autonomous replicating plasmid with an origin of replication). Additionally, included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acids in the vector are under the control of, and operably linked to, an appropriate promoter or other regulatory elements for transcription in a host cell. In some embodiments, the vector is a bi-functional expression vector which functions in multiple hosts.

### Codon Optimization

In some embodiments, the nucleic acid sequence is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species-specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleic acid sequences of this disclosure are codon optimized for expression in the organism/species of interest.

### Transformation

In some embodiments, the nucleic acid sequence, and/or expression cassettes disclosed herein are expressed transiently and/or stably incorporated into the genome of a host organism. In some embodiments, a the nucleic acid sequence and/or expression cassettes disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleic acid sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

### Insertion Sites

In some embodiments, the insertion of the nucleic acid sequence into a bacteriophage preserves the lytic activity of the bacteriophage. In some embodiments, the nucleic acid sequence is inserted into the bacteriophage genome. In some embodiments, the nucleic acid sequence is inserted into the bacteriophage genome at a transcription terminator site at the end of an operon of interest. In some embodiments, the nucleic acid sequence is inserted into the bacteriophage genome as a replacement for one or more removed non-essential genes. In some embodiments, the nucleic acid sequence is inserted into the bacteriophage genome as a replacement for one or more removed lysogenic genes. In some embodiments, the replacement of non-essential and/or lysogenic genes with the nucleic acid sequence does not affect the lytic activity of the bacteriophage. In some embodiments, the replacement of non-essential and/or lysogenic genes with the nucleic acid sequence preserves the lytic activity of the bacteriophage. In some embodiments, the replacement of non-essential and/or lysogenic genes with the nucleic acid sequence enhances the lytic activity of the bacteriophage. In some embodiments, the replacement of non-essential and/or lysogenic genes with the nucleic acid sequence renders a lysogenic bacteriophage lytic.

In some embodiments, the nucleic acid sequence is introduced into the bacteriophage genome at a first location while one or more non-essential and/or lysogenic genes are separately removed and/or inactivated from the bacteriophage genome at a separate location. In some embodiments, the nucleic acid sequence is introduced into the bacteriophage at a first location while one or more non-essential and/or lysogenic genes are separately removed and/or inactivated from the bacteriophage genome at multiple separate locations. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage.

In some embodiments, the nucleic acid sequence is introduced into the bacteriophage genome at a first location. In some embodiments, the nucleic acid sequence is introduced into the bacteriophage genome at a first location and a second location. In some embodiments, the nucleic acid sequence is introduced into the bacteriophage genome at a first location, a second location, and a third location. In some embodiments, the nucleic acid sequence is introduced into the bacteriophage in at least 1 location, 2 locations, 3 locations, 4 locations, 5 locations, 6 locations, 7 locations, 8 locations, 9 locations, or 10 locations. In some embodiments, the nucleic acid sequence is introduced into the bacteriophage in up to 10 locations, 20 locations, 30 locations, 40 locations, 50 locations, 60 locations, 70 locations, 80 locations, 90 locations, or 100 locations. In some embodiments, the nucleic acid sequence is introduced into the bacteriophage genome in a location under the control of an early promoter. In some embodiments, the nucleic acid sequence is introduced into the bacteriophage genome in a location under the control of a middle promoter. In some embodiments, the nucleic acid sequence is introduced into the bacteriophage genome in a location under the control of a late promoter. In some embodiments, the nucleic acid sequence is introduced into the bacteriophage genome in multiple locations under the control of an early promoter, a middle promoter, and/or a late promoter. In some embodiments, “early promoter” refers to a promoter that drives expression in an early phase of viral infection. In some embodiments, a “middle promoter” refers to a promoter that drives expression in an intermediate phase of viral infection. In some embodiments, a middle promoter drives expression at a later phase of viral infection than an early promoter. In some embodiments, a “late promoter” refers to a promoter that drives expression in a late phase of viral infection. In some embodiments, a “late promoter” refers to any promoter that is active after viral DNA replication has occurred. In some embodiments, a late promoter refers to a promoter that is only active if no substance is added that blocks DNA replication. In some embodiments, a late promoter drives expression at a later phase of viral infection than a middle promoter.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

### Non-Essential Gene

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is the hoc gene from a T4 E. coli bacteriophage. In some embodiments, the non-essential gene to be removed and/or replaced include gp0.7, gp4.3, gp4.5, gp4.7, or any combination thereof from a T7 E. coli bacteriophage. In some embodiments, the non-essential gene to be removed and/or replaced is gp0.6, gp0.65, gp0.7, gp4.3, gp4.5, or any combination thereof from a T7m E. coli bacteriophage.

### Methods of Use

Disclosed herein, in certain embodiments, are methods of expressing a therapeutic peptide in a target bacterium. Further disclosed herein, in certain embodiments, are methods for killing a target bacterium. Also disclosed herein, in certain embodiments, are methods of treating a disease in an individual in need thereof, the method comprising administering to the individual any of the bacteriophages disclosed herein.

In certain aspects, disclosed herein is a method of producing an anti-inflammatory peptide in a subject, the method comprising, infecting a bacterial cell with a bacteriophage comprising a nucleic acid, wherein the nucleic acid comprises a sequence encoding the therapeutic peptide and an operable lytic gene sequence, producing the therapeutic peptide within the bacterial cell, and releasing the therapeutic peptide by lysis of the bacterial cell, where the lysis is regulated by the operable lytic gene sequence. In some aspects, the therapeutic peptide is continually produced when the target bacteria are present.

In certain aspects, disclosed herein is a method of treating a disease or a disorder in the subject comprising administering a recombinant lytic bacteriophage configured to infect a bacterial cell within a subject, wherein the bacteriophage comprises a nucleic acid, wherein the nucleic acid comprises a sequence encoding the therapeutic peptide and an operable lytic gene sequence; producing the therapeutic peptide in a bacterial cell in a population of bacterial cells; and lysing the bacterial cell. In certain embodiments, the lysis is regulated by the operable lytic gene sequence. In certain embodiments, the production of therapeutic peptide in a bacterial cell is regulated by modulating the latent period. In certain embodiments, the population of bacterial cells fluctuate between upper and lower limits. In some embodiments, the lower limit does not equal zero CFUs. In some embodiments, there is no upper limit. In some embodiments, the upper limit comprises at least 2 times, 3 times, 4 times, 5 times, 6 times, 7 times, 8 times, 9 times, 10 times, or more than 10 times the number of bacterial cells as the lower limit. In some embodiments, the upper limit comprises at least 100 times; 1,000 times; 10,000 times; 100,000 times; 1,000,000 times; 10,000,000 times; 100,000,000 times; 1,000,000,000 times; or more than 1,000,000,000 times the number of bacterial cells as the lower limit.

In some embodiments, the therapeutic peptide is encoded by the recombinant bacteriophage. In some embodiments, the therapeutic peptide is expressed in a target bacterium. In some embodiments, therapeutic peptide is released from the target bacterium. In some embodiments, the therapeutic peptide is released from the target bacterium through the lytic activity of the recombinant bacteriophage. In some embodiments, the therapeutic peptide comprises an anti-inflammatory interleukin. In some embodiments, the anti-inflammatory interleukin is IL-10. In some embodiments, the anti-inflammatory interleukin is IL-4. In some embodiments, the anti-inflammatory interleukin is IL-6. In some embodiments, the anti-inflammatory interleukin is IL-9. In some embodiments, the anti-inflammatory interleukin is IL-11. In some embodiments, the anti-inflammatory interleukin is IL-13. In some embodiments, the anti-inflammatory interleukin is IL-19. In some embodiments, the anti-inflammatory interleukin is IL-27. In some embodiments, the anti-inflammatory interleukin is IL-35. In some embodiments, the anti-inflammatory interleukin is IL-37. In some embodiments, the therapeutic peptide comprises an anti-inflammatory peptide. In some embodiments, the anti-inflammatory peptide comprises pancreatitis-associated protein, Mycobacterium leprae Hsp65, bioactive heme oxygenase 1, EDSGTT or TNFα Receptor peptide (TNFRI) peptide.

In some embodiments, the therapeutic peptide interacts with at least one cell of the subject. In some embodiments, the cell is an immune cell. In some embodiments, the immune cell comprises a T cell, a B cell, a Natural Killer cell, a macrophage, a dendritic cell, an eosinophil, a neutrophil, a basophil, or a lymphocyte. In some embodiments, the therapeutic peptide interacts with a leukocyte. In some embodiments, the immune cell comprises a macrophage. In some embodiments, the immune cell comprises a T-cell. In some embodiments the therapeutic peptide interacts with an epithelial cell. In some embodiments, the therapeutic peptide modulates an immune response in the subject. In some embodiments, the therapeutic peptide modulates a reduction in inflammatory immune signaling. In some embodiments, the cell is an epithelial cell. In some embodiments, the cell is an endothelial cell. In some embodiments, the cell is an adipose cell. In some embodiments, the cell is a fibroblast. In some embodiments, the therapeutic peptide interacts with acellularized connective tissue. In some embodiments, the therapeutic peptide interacts with extracellular matrices.

In some embodiments, the therapeutic peptide affects an immune signaling pathway. In some embodiments, the therapeutic peptide affects an inflammatory signaling pathway. In some embodiments, the therapeutic peptide affects TNF-signaling. In some embodiments, the therapeutic peptide affects IL-1 signaling. In some embodiments, the therapeutic peptide affects IL-6 signaling. In some embodiments, the therapeutic peptide affects IL-4 signaling. In some embodiments, the therapeutic peptide affects IL-13 signaling. In some embodiments, the therapeutic peptide affects IL-2 signaling. In some embodiments, the therapeutic peptide affects TGF-signaling. In some embodiments, the therapeutic peptide affects EGF signaling. In some embodiments, the therapeutic peptide affects HGH signaling. In some embodiments, the therapeutic peptide affects Insulin signaling. In some embodiments, the therapeutic peptide affects IGF signaling. In some embodiments, the therapeutic peptide affects NGF signaling. In some embodiments, the therapeutic peptide affects ROS1 signaling. In some embodiments, the therapeutic peptide affects ALK signaling. In some embodiments, the therapeutic peptide affects IFN signaling. In some embodiments, the therapeutic peptide affects IDO signaling. In some embodiments, the therapeutic peptide affects PD-1 signaling. In some embodiments, the therapeutic peptide affects PD-L1 signaling. In some embodiments, the therapeutic peptide affects CTLA-4 signaling. In some embodiments, the therapeutic peptide affects LAG-3 signaling. In some embodiments, the therapeutic peptide affects VISTA signaling. In some embodiments, the therapeutic peptide affects TIM-3 signaling. In some embodiments, the therapeutic peptide affects MMP signaling. In some embodiments, the therapeutic peptide affects VEGF signaling. In some embodiments, the therapeutic peptide affects Wnt signaling.

In some embodiments, the antibody is encoded by the recombinant bacteriophage. In some embodiments, the antibody is expressed in a target bacterium. In some embodiments, the antibody is released from the target bacterium. In some embodiments, the antibody is released from the target bacterium through the lytic activity of the recombinant bacteriophage

In some embodiments, the antibody interacts with at least one peptide of the subject. In some embodiments, the antibody interacts with at least one protein of the subject. In some embodiments, the antibody interacts with a soluble protein. In some embodiments, the antibody interacts with an extracellular protein. In some embodiments, the antibody interacts with at least one growth factor of the subject. In some embodiments, the antibody interacts with at least one growth factor receptor of the subject. In some embodiments, the antibody interacts with at least one cytokine of the subject. In some embodiments, the antibody interacts with at least one cytokine receptor of the subject. In some embodiments, the antibody interacts with at least one hormone of the subject. In some embodiments, the antibody interacts with at least one hormone receptor of the subject. In some embodiments, the antibody interacts with at least one interleukin of the subject. In some embodiments, the antibody interacts with at least one interleukin receptor of the subject. In some embodiments, the antibody interacts with at least one integrin receptor of the subject. In some embodiments, the antibody interacts with at least one adhesion molecule of the subject. In some embodiments, the antibody modulates an immune response in the subject. In some embodiments, the antibody modulates a reduction in inflammatory immune signaling.

In some embodiments, the target bacterium is killed solely by lytic activity of the bacteriophage. In some embodiments, the lytic activity of the bacteriophage is modulated to favor more rapid killing through the lytic activity of the bacteriophage. In some embodiments, the lytic activity of the bacteriophage is modulated to disfavor more rapid killing to encourage the expression of the therapeutic peptide.

In some embodiments, the expression of the therapeutic peptide is modulated to favor increased expression of the therapeutic peptide. In some embodiments, the expression of the therapeutic peptide is modulated to disfavor increased expression of the therapeutic peptide.

In some embodiments, the recombinant bacteriophage is replication competent. In some embodiments, the replication competency is modulated to favor a slower replication process. In some embodiments, the replication competency is modulated to disfavor a slower replication process. In some embodiments, the replication competency is modulated to favor an increase in the number of recombinant bacteriophage produced per target bacteria. In some embodiments, the replication competency is modulated to disfavor an increase in the number of recombinant bacteriophage produced per target bacteria. In some embodiments, the recombinant bacteriophage is not replication competent.

### Lokta-Volterra Dynamics

Traditional bacteriophage therapy relies upon the delivery of a high-dosage treatment to a site of interest with the goal of rapidly eliminating a target bacterial population. A novel application for phage therapy leverages predator-prey population dynamics (modeled by the Lotka-Volterra equations) to facilitate the co-existence of a bacterial and phage population, leading to the long-term, continual production of a phage therapeutic. If the phages are engineered to express a therapeutic payload, a steady-state in vivo delivery of that payload can be achieved.

The Lotka-Volterra equations model the cyclical population dynamics observed in a classical predator-prey relationship, wherein the abundance of both populations is directly affected by the other. For instance, as the numbers of prey increases, the number of predators that can be supported similarly rises. However, once the predator population reaches too high of an abundance, there is a crash in the prey population as they are consumed. Due to the sudden reduction in available prey resources, the predator population also experiences a substantial decline. This cycle then repeats indefinitely unless an outside force drives one of the two populations to extinction.

The initiation of a predator-prey cycle requires a much lower, non-obvious initial dosage of phage than would a traditional treatment intended to wipe-out the target bacterial population. Furthermore, the target bacterial population would not be limited to a pathogenic or deleterious one (as that should be eradicated as quickly as possible), but rather could include a commonly occurring symbiont in the gut microbiome or elsewhere in the body (e.g., tumor microenvironment).

In certain embodiments, the population of bacterial cells fluctuate between upper and lower limits. In certain embodiments, the lower limit is greater than zero CFUs. In certain embodiments, there is no upper limit.

In summary, in some embodiments the goal is to establish a deliberate co-existence of the phage and target bacterial populations in order to achieve continual, long-term, site-localized expression of an engineered payload.

In some embodiments, provided herein is a method of producing a therapeutic peptide in a host organism, comprising contacting the host organism with a lytic bacteriophage comprising a nucleic acid encoding the therapeutic peptide; producing the therapeutic peptide within one or more of a plurality of target bacterial cells present in the host organism; and releasing the therapeutic peptide by lysis of the one or more target bacterial cells. In some embodiments, the bacteriophage and the plurality of the target bacterial cells co-exist in the host organism for at least about 1 day. In some embodiments, the therapeutic peptide is released for at least about 1 day. In some embodiments, the bacteriophage and the plurality of the target bacterial cells co-exist in the host organism for at least about 1 day, and the therapeutic peptide is released for at least about 1 day. In some cases, at least about one day includes at least or about 1, 2, 3, 4, 5, 6, or 7 days; at least or about 1 week, 2 weeks, 3 weeks, or 4 weeks; at least or about 1 month, 2 months, 3 months, 4 months, 5 months, 6 months, 7 months, 8 months, 9 months, 10 months, 11 months, or 12 months; and at least or about 1 year. In some cases, at least about one day is at least or about 1 day, and up to about 1 year, 2 years, 3 years, 4 years, or 5 years. In some embodiments, the bacteriophage and the plurality of the target bacterial cells co-exist in the host organism for at least about 2 days, and/or the therapeutic peptide is released for at least about 2 days. In some embodiments, the bacteriophage and the plurality of the target bacterial cells co-exist in the host organism for at least about 3 days, and/or the therapeutic peptide is released for at least about 3 days. In some embodiments, the bacteriophage and the plurality of the target bacterial cells co-exist in the host organism for at least about 4 days, and/or the therapeutic peptide is released for at least about 4 days. In some embodiments, the bacteriophage and the plurality of the target bacterial cells co-exist in the host organism for at least about 5 days, and/or the therapeutic peptide is released for at least about 5 days. In some embodiments, the bacteriophage and the plurality of the target bacterial cells co-exist in the host organism for at least about 6 days, and/or the therapeutic peptide is released for at least about 6 days. In some embodiments, the bacteriophage and the plurality of the target bacterial cells co-exist in the host organism for at least about 7 days, and/or the therapeutic peptide is released for at least about 7 days. In some embodiments, the bacteriophage and the plurality of the target bacterial cells co-exist in the host organism for at least about 1 week, and/or the therapeutic peptide is released for at least about 1 week. In some embodiments, the bacteriophage and the plurality of the target bacterial cells co-exist in the host organism for at least about 2 weeks, and/or the therapeutic peptide is released for at least about 2 weeks. In some embodiments, the bacteriophage and the plurality of the target bacterial cells co-exist in the host organism for at least about 3 weeks, and/or the therapeutic peptide is released for at least about 3 weeks. In some embodiments, the bacteriophage and the plurality of the target bacterial cells co-exist in the host organism for at least about 4 weeks, and/or the therapeutic peptide is released for at least about 4 weeks. In some embodiments, the bacteriophage and the plurality of the target bacterial cells co-exist in the host organism for at least about 1 month, and/or the therapeutic peptide is released for at least about 1 month. In some embodiments, the bacteriophage and the plurality of the target bacterial cells co-exist in the host organism for at least about 2 months, and/or the therapeutic peptide is released for at least about 2 months. In some embodiments, the bacteriophage and the plurality of the target bacterial cells co-exist in the host organism for at least about 3 months, and/or the therapeutic peptide is released for at least about 3 months. In some embodiments, co-existence occurs with the progeny of the phage that was contacted with the host organism, and the progeny of the bacteria that was present in the host organism at the time of contact.

In some embodiments, provided herein is a method of producing a therapeutic peptide in a host organism, comprising contacting the host organism with a lytic bacteriophage comprising a nucleic acid encoding the therapeutic peptide; producing the therapeutic peptide within one or more of a plurality of target bacterial cells present in the host organism; and releasing the therapeutic peptide by lysis of the one or more target bacterial cells. In some embodiments, the lytic bacteriophage is contacted with the host organism in an amount that produces a therapeutically effective amount of the therapeutic peptide without eliminating the plurality of the target bacterial cells. In some embodiments, the lytic bacteriophage is contacted with the host organism in an amount that produces a therapeutically effective amount of the therapeutic peptide, and the target bacterial cells remain above the limit of detection for at least about 1 day after contact with the host organism. In some cases, at least about one day includes at least or about 1, 2, 3, 4, 5, 6, or 7 days; at least or about 1 week, 2 weeks, 3 weeks, or 4 weeks; at least or about 1 month, 2 months, 3 months, 4 months, 5 months, 6 months, 7 months, 8 months, 9 months, 10 months, 11 months, or 12 months; and at least or about 1 year. In some cases, at least about one day is at least or about 1 day, and up to about 1 year, 2 years, 3 years, 4 years, or 5 years. In some embodiments, the lytic bacteriophage is contacted with the host organism in an amount that produces a therapeutically effective amount of the therapeutic peptide, and the target bacterial cells remain above the limit of detection for at least about 2 days after contact with the host organism, and/or the therapeutic peptide is released for at least about 2 days. In some embodiments, the lytic bacteriophage is contacted with the host organism in an amount that produces a therapeutically effective amount of the therapeutic peptide, and the target bacterial cells remain above the limit of detection for at least about 3 days after contact with the host organism, and/or the therapeutic peptide is released for at least about 3 days. In some embodiments, the lytic bacteriophage is contacted with the host organism in an amount that produces a therapeutically effective amount of the therapeutic peptide, and the target bacterial cells remain above the limit of detection for at least about 4 days after contact with the host organism, and/or the therapeutic peptide is released for at least about 4 days. In some embodiments, the lytic bacteriophage is contacted with the host organism in an amount that produces a therapeutically effective amount of the therapeutic peptide, and the target bacterial cells remain above the limit of detection for at least about 5 days after contact with the host organism, and/or the therapeutic peptide is released for at least about 5 days. In some embodiments, the lytic bacteriophage is contacted with the host organism in an amount that produces a therapeutically effective amount of the therapeutic peptide, and the target bacterial cells remain above the limit of detection for at least about 6 days after contact with the host organism, and/or the therapeutic peptide is released for at least about 6 days. In some embodiments, the lytic bacteriophage is contacted with the host organism in an amount that produces a therapeutically effective amount of the therapeutic peptide, and the target bacterial cells remain above the limit of detection for at least about 7 days after contact with the host organism, and/or the therapeutic peptide is released for at least about 7 days. In some embodiments, the lytic bacteriophage is contacted with the host organism in an amount that produces a therapeutically effective amount of the therapeutic peptide, and the target bacterial cells remain above the limit of detection for at least about 1 month after contact with the host organism, and/or the therapeutic peptide is released for at least about 1 month. In some embodiments, the lytic bacteriophage is contacted with the host organism in an amount that produces a therapeutically effective amount of the therapeutic peptide, and the target bacterial cells remain above the limit of detection for at least about 2 months after contact with the host organism, and/or the therapeutic peptide is released for at least about 2 months. In some embodiments, the lytic bacteriophage is contacted with the host organism in an amount that produces a therapeutically effective amount of the therapeutic peptide, and the target bacterial cells remain above the limit of detection for at least about 3 months after contact with the host organism, and/or the therapeutic peptide is released for at least about 3 months. In some embodiments, the lytic bacteriophage is contacted with the host organism in an amount that produces a therapeutically effective amount of the therapeutic peptide, and the target bacterial cells remain above the limit of detection for at least about 1 year after contact with the host organism, and/or the therapeutic peptide is released for at least about 1 year.

In some embodiments, provided herein is a method of producing a therapeutic peptide in a host organism, comprising contacting the host organism with a lytic bacteriophage comprising a nucleic acid encoding the therapeutic peptide; producing the therapeutic peptide within one or more of a plurality of target bacterial cells present in the host organism; and releasing the therapeutic peptide by lysis of the one or more target bacterial cells. In some embodiments, the bacteriophage and the plurality of target bacterial cells co-exist in the host organism for at least about 1 day after contact. In some embodiments, the therapeutic peptide is released for at least about 1 day after contact. In some embodiments, a therapeutically effective amount of the therapeutic peptide is produced without eliminating the plurality of the target bacterial cells. In some embodiments, a therapeutically effective amount of the therapeutic peptide is produced, and the plurality of target bacterial cells remains above the limit of detection for at least about 1 day after contact. In some embodiments, detection is based on presence of colonies after plating on an agar plate. In some embodiments, the agar plate comprises a Luria Broth (LB) plate. In some cases, at least about one day includes at least or about 1, 2, 3, 4, 5, 6, or 7 days; at least or about 1 week, 2 weeks, 3 weeks, or 4 weeks; at least or about 1 month, 2 months, 3 months, 4 months, 5 months, 6 months, 7 months, 8 months, 9 months, 10 months, 11 months, or 12 months; and at least or about 1 year. In some cases, at least about one day is at least or about 1 day, and up to about 1 year, 2 years, 3 years, 4 years, or 5 years.

### Lytic Lifestyle Latent Period

The latent period is the time taken by a bacteriophage to reproduce inside an infected bacterium and erupt from the lysed cell. While several factors contribute to the length of the latent period, the ability of the phage to lyse the bacterium is a key contributor to the length of this time period. During the lytic lifecycle, bacteriophages will produce two proteins. One is a murein hydrolase, or lysin, and the other is a membrane protein, or holin. The function of the holin is to create a lesion in the cytoplasmic membrane through which the lysin passes to gain access to the murein layer, which ultimately leads to cytoplasmic instability and lysis.

While inhibition of lysins and/or holins does not prevent the ultimate lysis of the bacterium, it can increase the latent period. In some embodiments, an increase in the latent period provides the infected bacterium time to produce more bacteriophage and/or express a recombinant protein encoded by the bacteriophage. In some embodiments, the recombinant protein is an anti-inflammatory peptide. In some embodiments, the amount of anti-inflammatory peptide is increased above a baseline. In some embodiments, the baseline comprises the amount of anti-inflammatory peptide that is released in a bacteriophage where the latent period is not modified.

In some embodiments, the expression of lysins are reduced. In some embodiments, the expression of lysins are inhibited entirely. In some embodiments, the expression of holins are reduced. In some embodiments, the expression of holins are inhibited entirely. In some embodiments, the expression of lysins and holins are reduced. In some embodiments, the expression of lysins and holins are inhibited entirely. In some embodiments, the reduced expression of lysins increases the latent period. In some embodiments, the reduced expression of holins increases the latent period. In some embodiments, the reduced expression of lysins and holins increases the latent period. In some embodiments, the increased latent period by reducing lysin expression allows for increased expression of a recombinant protein. In some embodiments, the increased latent period by reducing holin expression allows for increased expression of a recombinant protein. In some embodiments, the increased latent period by reducing lysin and holin expression allows for increased expression of a recombinant protein.

### Disease or Disorder

In some embodiments, the lytic activity of the recombinant bacteriophage described herein is used to kill a target bacterium causing a disease or disorder. In some embodiments, the lytic activity of the recombinant bacteriophage is used to kill a target bacterium exacerbating a disease. In some embodiments, the lytic activity of the recombinant bacteriophage is used to kill a target bacterium causing an inflammatory process. In some embodiments, the lytic activity of the recombinant bacteriophage is used kill a target bacterium exacerbating an inflammatory process. In some embodiments, the lytic activity of the recombinant bacteriophage is used to kill a target bacterium modulating the disease process in Immunology. In some embodiments, the lytic activity of the recombinant bacteriophage is used to kill a target bacterium modulating the disease process in Inflammatory Bowel Disease. In some embodiments, the lytic activity of the recombinant bacteriophage is used to kill a target bacterium modulating the disease process in Crohn's Disease. In some embodiments, the lytic activity of the recombinant bacteriophage is used to kill a target bacterium modulating the disease process in Ulcerative Colitis. In some embodiments, the lytic activity of the recombinant bacteriophage is used to kill a target bacterium modulating the disease process in Rheumatoid Arthritis. In some embodiments, the lytic activity of the recombinant bacteriophage is used to kill a target bacterium modulating the disease process in Psoriasis. In some embodiments, the lytic activity of the recombinant bacteriophage is used to kill a target bacterium modulating the disease process in Psoriatic Arthritis. In some embodiments, the lytic activity of the recombinant bacteriophage is used to kill a target bacterium modulating the disease process in Systemic Lupus Erythematosus. In some embodiments, the lytic activity of the recombinant bacteriophage is used to kill a target bacterium modulating the disease process in Lupus Nephritis. In some embodiments, the lytic activity of the recombinant bacteriophage is used to kill a target bacterium modulating the disease process in cancer. In some embodiments, the lytic activity of the recombinant bacteriophage is used to kill a target bacterium modulating a disease process in the nervous system.

In some embodiments, the expression of the therapeutic peptide is used to treat a disease. In some embodiments, the expression of the therapeutic peptide is used to treat an inflammatory process. In some embodiments, the expression of the therapeutic peptide is used to modulate a disease process in the immune system. In some embodiments, the expression of the therapeutic peptide is used to modulate a disease process. In some embodiments, the expression of the therapeutic peptide is used to modulate a disease process in Inflammatory Bowel Disease. In some embodiments, the expression of the therapeutic peptide is used to modulate a disease process in Crohn's Disease. In some embodiments, the expression of the therapeutic peptide is used to modulate a disease process in Ulcerative Colitis. In some embodiments, the expression of the therapeutic peptide is used to modulate a disease process in Rheumatoid Arthritis. In some embodiments, the expression of the therapeutic peptide is used to modulate a disease process in Psoriasis. In some embodiments, the expression of the therapeutic peptide is used to modulate a disease process in Psoriatic Arthritis. In some embodiments, the expression of the therapeutic peptide is used to modulate a disease process in Systemic Lupus Erythematosus. In some embodiments, the expression of the therapeutic peptide is used to modulate a disease process in Lupus Nephritis. In some embodiments, the expression of the therapeutic peptide is used to modulate a disease process in cancer. In some embodiments, the expression of the therapeutic peptide is used to modulate a disease process in the nervous system.

In some embodiments, the synergistic killing of the target bacterium and expression of the therapeutic peptide is used to treat a disease. In some embodiments, the disease is immunologic. In some embodiments, the disease is Inflammatory Bowel Disease. In some embodiments, the disease is Ulcerative Colitis. In some embodiments the disease is Crohn's Disease. In some embodiments, the disease is Rheumatoid Arthritis. In some embodiments, the disease is Psoriasis. In some embodiments the disease is Psoriatic Arthritis. In some embodiments, the disease is Systemic Lupus Erythematosus. In some embodiments, the disease is Lupus Nephritis. In some embodiments, the disease is cancer. In some embodiments, the disease is a disease of the nervous system. In some embodiments, the disease is an auto-immune disease.

### Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the microbial material comprises Proteobacteria, Actinobacteria, Bacteroidetes, or Firmicutes.

In some embodiments, the bacteriophages as disclosed herein are used to modulate or kill target bacteria within the microbiome of a subject. In certain embodiments, the bacterial species is a bacterial species that blooms during a flare of the disease or disorder described herein. In certain embodiments, the bacterial species comprise E. coli, K. pneumoniae, R. gnavus, E. gallinarum, E. faecalis, E. faecium, and B. fragilis. In some embodiments, the target bacterium is E. coli. In some embodiments, the target bacterium is an adherent-invasive E. coli (AEIC). In some embodiments, the E. coli is a multidrug-resistant (MDR) strain. In some embodiments, the E. coli is an extended spectrum beta-lactamase (ESBL) expressing strain. In some embodiments, the E. coli is a carbapenem-resistant strain. In some embodiments, the E. coli is a non-multidrug-resistant (non-MDR) strain. In some embodiments, the E. coli is a non-carbapenem-resistant strain. In some embodiments, the pathogenic bacteria are diarrheagenic. In some embodiments, the pathogenic bacteria are diarrheagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are enteropathogenic. In some embodiments, the pathogenic bacterium is enteropathogenic E. coli (EPEC).

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary bacteria associated with diseases and conditions of gastrointestinal tract and are being modulated or killed by the bacteriophages include strains, sub-strains, and enterotypes of E. coli.

In some embodiments, the bacteriophages are used to modulate or kill target single or plurality of bacteria within the microbiome or gut flora of the gastrointestinal tract of a subject. Disruption (e.g., dysbiosis) of the microbiome or gut flora increases the risk for health conditions such as diabetes, mental disorders, Crohn's disease, ulcerative colitis, colorectal cancer, autoimmune disorders, obesity, diabetes, and diseases of the central nervous system. An exemplary list of the bacteria associated with diseases and conditions of gastrointestinal tract and are being modulated or killed by the bacteriophages include strains, sub-strains, and enterotypes of Enterobacteriaceae, Pasteurellaceae, Fusobacteriaceae, Neisseriaceae, Veillonellaceae, Gemellaceae, Bacteriodales, Clostridiales, Erysipelotrichaceae, Bifidobacteriaceae, Bacteroides, Faecalibacterium, Roseburia, Blautia, Ruminococcus, Coprococcus, Streptococcus, Dorea, Blautia, Ruminococcus, Lactobacillus, Enterococcus, Streptococcus, Actinomyces, Lactococcus, Roseburia, Blautia, Dialister, Desulfovibrio, Escherichia, Lactobacillus, Coprococcus, Clostridium, Bifidobacterium, Klebsiella, Granulicatella, Eubacterium, Anaerostipes, Parabacteroides, Coprobacillus, Gordonibacter, Collinsella, Bacteroides, Faecalibacterium, Anaerotruncus, Alistipes, Haemophilus, Anaerococcus, Veillonella, Arevotella, Akkermansia, Bilophila, Sutterella, Eggerthella, Holdemania, Gemella, Peptoniphilus, Rothia, Pediococcus, Citrobacter, Odoribacter, Enterobacteria, Fusobacterium, Proteus, Escherichia coli, Fusobacterium nucleatum, Haemophilus parainfluenzae (Pasteurellaceae), Veillonella parvula, Eikenella corrodens (Neisseriaceae), Gemella moribillum, Bacteroides vulgatus, Bacteroides caccae, Bifidobacterium bifidum, Bifidobacterium longum, Bifidobacterium adolescentis, Bifidobacterium dentum, Blautia hansenii, Ruminococcus gnavus, Clostridium nexile, Faecalibacterium prausnitzii, Ruminoccus torques, Clostridium bolteae, Eubacterium rectale, Roseburia intestinalis, and Coprococcus iomes.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic or a third agent. In some embodiment, microbiome related disease or disorder is treated by a bacteriophage disclosed herein.

### Administration Routes and Dosage

Dose and duration of the administration of a composition disclosed herein will depend on a variety of factors, including the subject's age, subject's weight, and tolerance of the phage. In some embodiments, a bacteriophage disclosed herein is administered to patients intra-arterially, intravenously, intraurethrally, intramuscularly, orally, subcutaneously, by inhalation, or any combination thereof. In some embodiments, a bacteriophage disclosed herein is administered to patients by oral administration. In some embodiments, a bacteriophage disclosed herein is administered to patients by topical, cutaneous, transdermal, transmucosal, implantation, sublingual, buccal, rectal, vaginal, ocular, otic, or nasal administration. In some embodiments, a bacteriophage disclosed herein is administered to patients by any combination of the aforementioned routes of administration.

In some embodiments, a dose of phage between 10³ and 10²⁰ PFU is given. In some embodiments, a dose of phage between 10³ and 10¹⁰ PFU is given. In some embodiments, a dose of phage between 10⁶ and 10²⁰ PFU is given. In some embodiments, a dose of phage between 10⁶ and 10¹⁰ PFU is given. For example, in some embodiments, the bacteriophage is present in a composition in an amount between 10³ and 10¹¹ PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU, or more. In some embodiments, the bacteriophage is present in a composition in an amount of less than 10¹ PFU. In some embodiments, the bacteriophage is present in a composition in an amount between 10¹ and 10⁸, 10⁴ and 10⁹, 10⁵ and 10¹⁰, or 10⁷, and 10¹¹ PFU.

In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some embodiments, a bacteriophage or a mixture is administered to a subject in need thereof every 2, 4, 6, 8, 10, 12, 14, 18, 20, 22, or 24 hours.

In some embodiments, the compositions (bacteriophage) disclosed herein are administered before, during, or after the occurrence of a disease or condition. In some embodiments, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Pharmaceutical Compositions

Disclosed herein, in certain embodiments, are pharmaceutical compositions comprising (a) the nucleic acid sequences as disclosed herein; and (b) a pharmaceutically acceptable excipient. Also disclosed herein, in certain embodiments, are pharmaceutical compositions comprising (a) the bacteriophages as disclosed herein; and (b) a pharmaceutically acceptable excipient. Further disclosed herein, in certain embodiments, are pharmaceutical compositions comprising (a) the compositions as disclosed herein; and (b) a pharmaceutically acceptable excipient.

In some embodiments, the disclosure provides pharmaceutical compositions and methods of administering the same to treat bacterial, archaeal infections or to disinfect an area. In some embodiments, the pharmaceutical composition comprises any of the reagents discussed above in a pharmaceutically acceptable carrier. In some embodiments, a pharmaceutical composition or method disclosed herein treats urinary tract infections (UTI) and/or inflammatory diseases (e.g. inflammatory bowel disease (IBD)). In some embodiments, a pharmaceutical composition or method disclosed herein treats Crohn's disease. In some embodiments, a pharmaceutical composition or method disclosed herein treats ulcerative colitis.

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiments, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostals and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried (lyophilized) condition requiring only the addition of the sterile liquid carrier, for example, saline or water for injection on immediately prior to use.

In some embodiments, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition is produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20, or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient is a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide, and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to Ethylenediaminetetraacetic acid (EDTA), citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatin; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol, and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate, and talc, or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, zylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, amino acids such as L-leucine, sugar esters of fatty acids, glycerides of fatty acids, or a combination thereof.

In some instances, a pharmaceutical formulation comprises an additional pharmaceutical agent.

### Embodiments

Disclosed herein are certain non-limiting embodiments:
1. A recombinant lytic bacteriophage configured to infect a bacterial cell, wherein the bacteriophage comprises a nucleic acid comprising:
   (a) a first sequence encoding an anti-inflammatory interleukin, wherein the bacteriophage capsid does not comprise the anti-inflammatory interleukin; and
   (b) a second nucleic acid sequence encoding an operable lytic gene capable of inducing lysis of a target bacterium during a lytic cycle of the recombinant lytic bacteriophage.
2. The recombinant lytic bacteriophage of embodiment [0120], wherein the recombinant lytic bacteriophage is replication competent.
3. The recombinant lytic bacteriophage of embodiment [0120], wherein the recombinant lytic bacteriophage is replication incompetent.
4. The recombinant lytic bacteriophage of any one of embodiments [0120]-3, wherein anti-inflammatory interleukin comprises IL-4, IL-6, IL-9, IL-10, IL-11, IL-13, IL-19, IL-27, IL-35, and/or IL-37.
5. The recombinant lytic bacteriophage of any one of embodiments [0120]-4, wherein the operable lytic gene sequence is derived from a bacteriophage sequence.
6. The recombinant lytic bacteriophage of any one of embodiments [0120]-4, wherein the operable lytic gene sequence regulates the lytic cycle of the bacteriophage.
7. The recombinant lytic bacteriophage of any one of embodiments [0120]-5, wherein the operable lytic gene sequence regulates the production of holin.
8. The recombinant lytic bacteriophage of any one of embodiments [0120]-108, wherein the bacteriophage is selected from a group consisting of the following genera: Alcyoneusvirus, Asteriusvirus, Bifseptvirus, Biseptimavirus, Bonnellvirus, Chivirus, Dhakavirus, Dhillonvirus, Drulisvirus, Epseptimavirus, Felixounavirus, Gamaleyavirus, Gequatrovirus, Goslavirus, Guelphvirus, Hanrivervirus, Inovirus, Jiaodavirus, Kagunavirus, Kayfunavirus, Krischvirus, Kuravirus, Lederbergvirus, Levivirus, Mosigvirus, Nonagvirus, Peduovirus, Phapecoctavirus, Przondovirus, Rogunavirus, Saphexavirus, Schiekvirus, Seunavirus, Seuratvirus, Skarprettervirus, Slopekvirus, Sugarlandvirus, Taipeivirus, Tequatrovirus, Tequintavirus, Teseptimavirus, Uetakavirus, Vectrevirus, Vequintavirus, Webervirus.
9. A method of producing an anti-inflammatory peptide in a subject comprising:
   (a) infecting a target bacterial cell with a bacteriophage comprising a nucleic acid, wherein the nucleic acid comprises a sequence encoding the anti-inflammatory peptide and an operable lytic gene sequence;
   (b) producing the anti-inflammatory peptide within the target bacterial cell;
   (c) releasing the anti-inflammatory peptide by lysis of the target bacterial cell, wherein the lysis is regulated by the operable lytic gene sequence;
wherein the anti-inflammatory peptide is continually produced if the target bacterial cell is present.
10. A method of producing an anti-inflammatory peptide in a subject comprising:
   (a) infecting a target bacterial cell with a bacteriophage comprising a nucleic acid, wherein the nucleic acid comprises a sequence encoding the interleukin and an operable lytic gene sequence;
   (b) producing the anti-inflammatory peptide within the target bacterial cell;
   (c) releasing the anti-inflammatory peptide by lysis of the target bacterial cell, wherein the latent period of the lytic cycle is increased by down-regulating holin production; whereby the amount of anti-inflammatory peptide released upon lysis is increased above a baseline.
11. The method of embodiments 9 or 10, wherein the anti-inflammatory peptide is soluble and is not expressed on a capsid protein of the recombinant bacteriophage.
12. The method of any one of embodiments 9-11, wherein the operable lytic gene sequence is derived from a bacteriophage sequence.
13. The method of any one of embodiments 9-12, wherein the operable lytic gene sequence regulates the lytic cycle of the bacteriophage.
14. The method of any one of embodiments 9-13, wherein the target bacterial cell comprises a bacterial species that bloom during a flare.
15. The method of embodiment 14, wherein the target bacterial cell comprises E. coli, K. pneumoniae, R. gnavus, E. gallinarum, E. faecalis, E. faecium, and/or B. fragilis.
16. The method of any one of embodiments 9-15, wherein the bacteriophage is recombinant, lytic, and replication competent.
17. The method of any one of embodiments 9-15, wherein the bacteriophage is recombinant, lytic, and replication incompetent.
18. The method of any one of embodiments 9-16, wherein the anti-inflammatory peptide is an anti-inflammatory interleukin.
19. The method of embodiment 18, wherein anti-inflammatory interleukin comprises TL-4, IL-6, IL-9, IL-10, IL-11, IL-13, IL-19, IL-27, IL-35, or IL-37.
20. The method of embodiment 18, wherein the interleukin interacts with at least one cell of the subject.
21. The method of embodiment 20, wherein the interleukin interacts with at least one immune cell of the subject.
22. The method of embodiment 20, wherein the interleukin interacts with at least one T cell of the subject.
23. The method of embodiment 20, wherein the interleukin interacts with at least one epithelial cell of the subject.
24. The method of embodiment 20, wherein the interleukin interacts with at least one macrophage of the subject.
25. The method of embodiment 20, wherein the interleukin modulates an immune response in the subject.
26. The method of embodiment 20, wherein the interleukin modulates a reduction in inflammatory immune signaling.
27. A method of treating a disease or disorder in a subject comprising:
   (a) administering a recombinant lytic bacteriophage configured to infect a target bacterial cell within a subject, wherein the bacteriophage comprises a nucleic acid, wherein the nucleic acid comprises a sequence encoding the anti-inflammatory peptide and an operable lytic gene sequence;
   (b) producing the anti-inflammatory peptide in a target bacterial cell in a population of bacterial cells;
   (c) lysing the target bacterial cell,
      wherein the lysis is regulated by the operable lytic gene sequence;
      wherein the population of bacterial cells fluctuate between upper and lower limits where the lower limit does not equal zero CFUs and there is no upper limit.
28. The method of embodiment 27, wherein the anti-inflammatory peptide is soluble and is not expressed on a capsid protein of the recombinant lytic bacteriophage comprising the operable lytic gene sequence.
29. The method of embodiment 27 or 28, wherein the operable lytic gene sequence is derived from a bacteriophage sequence.
30. The method of any one of embodiments 27-29, wherein the operable lytic gene sequence regulates the lytic cycle of the bacteriophage.
31. The method of any one of embodiments 27-30, wherein the operable lytic gene sequence regulates the production of holin.
32. The method of embodiment 27-31, wherein the target bacterial cell comprises a bacterial species that bloom during a flare.
33. The method of embodiment 32, wherein the target bacterial cell comprises E. coli, K. pneumoniae, R. gnavus, E. gallinarum, E. faecalis, E. faecium, or B. fragilis.
34. The method of any one of embodiments 27-33, wherein the recombinant lytic bacteriophage is replication competent.
35. The method of any one of embodiments 27-33, wherein the recombinant lytic bacteriophage is replication incompetent.
36. The method of any one of embodiments 27-35, wherein the anti-inflammatory peptide is an anti-inflammatory interleukin.
37. The method of embodiment 36, wherein anti-inflammatory interleukin comprises IL-4, IL-6, IL-9, IL-10, IL-11, IL-13, IL-19, IL-27, IL-35, or IL-37.
38. The method of embodiment 27-37, wherein the disease or disorder comprises Inflammatory Bowel Disease, Ulcerative Colitis, Crohn's Disease, Rheumatoid Arthritis, Psoriasis, Psoriatic Arthritis, Systemic Lupus Erythematosus, Lupus Nephritis, cancer, or diseases of the nervous system.
39. A recombinant lytic bacteriophage configured to infect a bacterial cell,
   wherein the bacteriophage comprises a nucleic acid comprising:
      (a) a first sequence encoding an antibody, wherein the bacteriophage capsid does not comprise the antibody; and
      (b) a second nucleic acid sequence encoding an operable lytic gene capable of inducing lysis of a target bacterium during a lytic cycle of the recombinant lytic bacteriophage.
40. The recombinant lytic bacteriophage of embodiment 39, wherein the recombinant lytic bacteriophage is replication competent.
41. The recombinant lytic bacteriophage of embodiment 39, wherein the recombinant lytic bacteriophage is replication incompetent.
42. The recombinant lytic bacteriophage of any one of embodiments 39-41, wherein the antibody is a nanobody.
43. The recombinant lytic bacteriophage of any one of embodiments 39-42, wherein the antibody affects signaling from a pathway selected from the group consisting of TNF-α signaling, IL-1 signaling, IL-6 signaling, IL-4 signaling, IL-13 signaling, IL-2 signaling, TGF-β signaling, EGF signaling, HGH signaling, IGF signaling, NGF signaling, ROS1 signaling, ALK signaling, IFNγ signaling, IDO signaling, PD-1 signaling, PD-L1 signaling, CTLA-4 signaling, LAG-3 signaling, VISTA signaling, TIM-3 signaling, MMP signaling, VEGF signaling, and Wnt signaling.
44. The recombinant lytic bacteriophage of any one of embodiments 39-43, wherein the operable lytic gene sequence is derived from a bacteriophage sequence.
45. The recombinant lytic bacteriophage of any one of embodiments 39-44, wherein the operable lytic gene sequence regulates the lytic cycle of the bacteriophage.
46. The recombinant lytic bacteriophage of embodiment 45, wherein the operable lytic gene sequence regulates the production of holin.
47. The recombinant lytic bacteriophage of any one of embodiments 39-46, wherein the bacteriophage is selected from a group consisting of the following genera: Alcyoneusvirus, Asteriusvirus, Bifseptvirus, Biseptimavirus, Bonnellvirus, Chivirus, Dhakavirus, Dhillonvirus, Drulisvirus, Epseptimavirus, Felixounavirus, Gamaleyavirus, Gequatrovirus, Goslavirus, Guelphvirus, Hanrivervirus, Inovirus, Jiaodavirus, Kagunavirus, Kayfunavirus, Krischvirus, Kuravirus, Lederbergvirus, Levivirus, Mosigvirus, Nonagvirus, Peduovirus, Phapecoctavirus, Przondovirus, Rogunavirus, Saphexavirus, Schiekvirus, Seunavirus, Seuratvirus, Skarprettervirus, Slopekvirus, Sugarlandvirus, Taipeivirus, Tequatrovirus, Tequintavirus, Teseptimavirus, Uetakavirus, Vectrevirus, Vequintavirus, Webervirus.
48. A method of producing an anti-inflammatory peptide in a subject comprising:
   (a) infecting a target bacterial cell with a bacteriophage comprising a nucleic acid, wherein the nucleic acid comprises a sequence encoding the anti-inflammatory peptide and an operable lytic gene sequence;
   (b) producing the anti-inflammatory peptide within the target bacterial cell;
   (c) releasing the anti-inflammatory peptide by lysis of the target bacterial cell,
   wherein the lysis is regulated by the operable lytic gene sequence;
wherein the anti-inflammatory peptide is continually produced if the target bacterial cell is present.
49. A method of producing an anti-inflammatory peptide in a subject comprising:
   (a) infecting a target bacterial cell with a bacteriophage comprising a nucleic acid, wherein the nucleic acid comprises a sequence encoding the interleukin and an operable lytic gene sequence;
      (b) producing the anti-inflammatory peptide within the target bacterial cell;
      (c) releasing the anti-inflammatory peptide by lysis of the bacterial cell, wherein the latent period of the lytic cycle is increased by down-regulating holin production; whereby the amount of anti-inflammatory peptide released upon lysis is increased above a baseline.
50. The method of embodiment 48 or 49, wherein the anti-inflammatory peptide is soluble and is not expressed on a capsid protein of the recombinant bacteriophage.
51. The method of any one of embodiments 48-50, wherein the operable lytic gene sequence is derived from a bacteriophage sequence.
52. The method of any one of embodiments 48-51, wherein the operable lytic gene sequence regulates the lytic cycle of the bacteriophage.
53. The method of any one of embodiments 48-52, wherein the target bacterial cell comprises a bacterial species that blooms during a flare.
54. The method of any one of embodiments 48-53, wherein the target bacterial cell comprises E. coli, K. pneumoniae, R. gnavus, E. gallinarum, E. faecalis, E. faecium, or B. fragilis.
55. The method of any one of embodiments 48-54, wherein the bacteriophage is recombinant, lytic, and replication competent.
56. The method of any one of embodiments 48-55, wherein the bacteriophage is recombinant, lytic, and replication incompetent.
57. The method of any one of embodiments 48-56, wherein the anti-inflammatory peptide is an antibody.
58. The method of embodiment 57, wherein the antibody comprises a nanobody.
59. The method of embodiment 57 or 58, wherein the antibody affects signaling from a pathway selected from the group consisting of TNF-α signaling, IL-1 signaling, IL-6 signaling, IL-4 signaling, IL-13 signaling, IL-2 signaling, TGF-β signaling, EGF signaling, HGH signaling, IGF signaling, NGF signaling, ROS1 signaling, ALK signaling, IFNγ signaling, IDO signaling, PD-1 signaling, PD-L1 signaling, CTLA-4 signaling, LAG-3 signaling, VISTA signaling, TIM-3 signaling, MMP signaling, VEGF signaling, and Wnt signaling.
60. A method of treating a disease or disorder in a subject comprising:
   (a) administering a recombinant lytic bacteriophage configured to infect a target bacterial cell within a subject, wherein the bacteriophage comprises a nucleic acid, wherein the nucleic acid comprises a sequence encoding the anti-inflammatory peptide and an operable lytic gene sequence;
   (b) producing the anti-inflammatory peptide in a target bacterial cell in a population of bacterial cells;
   (c) lysing the target bacterial cell,
   wherein the lysis is regulated by the operable lytic gene sequence;
   wherein the population of bacterial cells fluctuate between upper and lower limits where the lower limit does not equal zero CFUs and there is no upper limit.
61. The method of embodiment 60, wherein the anti-inflammatory peptide is soluble and is not expressed on a capsid protein of the recombinant lytic bacteriophage comprising the operable lytic gene sequence.
62. The method of embodiment 60 or 61, wherein the operable lytic gene sequence is derived from a bacteriophage sequence.
63. The method of any one of embodiments 60-62, wherein the operable lytic gene sequence regulates the lytic cycle of the bacteriophage.
64. The method of any one of embodiments 60-63, wherein the operable lytic gene sequence regulates the production of holin.
65. The method of embodiment 60, wherein the target bacterial cell comprises a bacterial species that blooms during a flare.
66. The method of embodiment 65, wherein the target bacterial cell comprises E. coli, K. pneumoniae, R. gnavus, E. gallinarum, E. faecalis, E. faecium, or B. fragilis.
67. The method of any one of embodiments 60-66, wherein the recombinant lytic bacteriophage is replication competent.
68. The method of any one of embodiments 60-67, wherein the recombinant lytic bacteriophage is replication incompetent.
69. The method of any one of embodiments 60-68, wherein the anti-inflammatory peptide is an antibody.
70. The method of embodiment 69, wherein the antibody comprises a nanobody.
71. The method of embodiment 69 or 70, wherein the antibody affects signaling from a pathway selected from the group consisting of TNF-α signaling, IL-1 signaling, IL-6 signaling, IL-4 signaling, IL-13 signaling, IL-2 signaling, TGF-β signaling, EGF signaling, HGH signaling, IGF signaling, NGF signaling, ROS1 signaling, ALK signaling, IFNγ signaling, IDO signaling, PD-1 signaling, PD-L1 signaling, CTLA-4 signaling, LAG-3 signaling, VISTA signaling, TIM-3 signaling, MMP signaling, VEGF signaling, and Wnt signaling.
72. The method of embodiment 60, wherein the disease or disorder comprises Inflammatory Bowel Disease, Ulcerative Colitis, Crohn's Disease, Rheumatoid Arthritis, Psoriasis, Psoriatic Arthritis, Systemic Lupus Erythematosus, Lupus Nephritis, or cancer.
73. A recombinant lytic bacteriophage configured to infect a bacterial cell,
   wherein the bacteriophage comprises a nucleic acid comprising:
      (a) a first sequence encoding an anti-inflammatory peptide, wherein the bacteriophage capsid does not comprise the anti-inflammatory peptide; and
      (b) a second nucleic acid sequence encoding an operable lytic gene capable of inducing lysis of a target bacterium during a lytic cycle of the recombinant lytic bacteriophage.
74. The recombinant lytic bacteriophage of embodiment 73, wherein the recombinant lytic bacteriophage is replication competent.
75. The recombinant lytic bacteriophage of embodiment 73, wherein the recombinant lytic bacteriophage is replication incompetent.
76. The recombinant lytic bacteriophage of any one of embodiments 73-75, wherein the anti-inflammatory peptide comprises pancreatitis-associated protein, Mycobacterium leprae Hsp65, bioactive heme oxygenase 1, EDSGTT, or TNFRI peptide.
77. The recombinant lytic bacteriophage of any one of embodiments 73-76, wherein the operable lytic gene sequence is derived from a bacteriophage sequence.
78. The recombinant lytic bacteriophage of any one of embodiments 73-77, wherein the operable lytic gene sequence regulates the lytic cycle of the bacteriophage.
79. The recombinant lytic bacteriophage of embodiment 78, wherein the operable lytic gene sequence regulates the production of holin.
80. The recombinant lytic bacteriophage of any one of embodiments 73-79, wherein the bacteriophage is selected from a group consisting of the following genera: Alcyoneusvirus, Asteriusvirus, Bifseptvirus, Biseptimavirus, Bonnellvirus, Chivirus, Dhakavirus, Dhillonvirus, Drulisvirus, Epseptimavirus, Felixounavirus, Gamaleyavirus, Gequatrovirus, Goslavirus, Guelphvirus, Hanrivervirus, Inovirus, Jiaodavirus, Kagunavirus, Kayfunavirus, Krischvirus, Kuravirus, Lederbergvirus, Levivirus, Mosigvirus, Nonagvirus, Peduovirus, Phapecoctavirus, Przondovirus, Rogunavirus, Saphexavirus, Schiekvirus, Seunavirus, Seuratvirus, Skarprettervirus, Slopekvirus, Sugarlandvirus, Taipeivirus, Tequatrovirus, Tequintavirus, Teseptimavirus, Uetakavirus, Vectrevirus, Vequintavirus, Webervirus.
81. A method of producing an anti-inflammatory peptide in a subject comprising:
   (a) infecting a target bacterial cell with a bacteriophage comprising a nucleic acid, wherein the nucleic acid comprises a sequence encoding the anti-inflammatory peptide and an operable lytic gene sequence;
   (b) producing the anti-inflammatory peptide within the target bacterial cell;
   (c) releasing the anti-inflammatory peptide by lysis of the target bacterial cell,
   wherein the lysis is regulated by the operable lytic gene sequence;
wherein the anti-inflammatory peptide is continually produced if the target bacterial cell is present.
82. A method of producing an anti-inflammatory peptide in a subject comprising:
   (a) infecting a target bacterial cell with a bacteriophage comprising a nucleic acid, wherein the nucleic acid comprises a sequence encoding the interleukin and an operable lytic gene sequence;
      (b) producing the anti-inflammatory peptide within the target bacterial cell;
      (c) releasing the anti-inflammatory peptide by lysis of the target bacterial cell, wherein the latent period of the lytic cycle is increased by down-regulating holin production; whereby the amount of anti-inflammatory peptide releases upon lysis is increased above baseline.
83. The method of embodiment 81 to 82, wherein the anti-inflammatory peptide is soluble and is not expressed on a capsid protein of the recombinant bacteriophage.
84. The method of any one of embodiments 81-83, wherein the operable lytic gene sequence is derived from a bacteriophage sequence.
85. The method of any one of embodiments 81-84, wherein the operable lytic gene sequence regulates the lytic cycle of the bacteriophage.
86. The method of any one of embodiments 81-85, wherein the target bacterial cell comprises a bacterial species that bloom during a flare.
87. The method of embodiment 86, wherein the target bacterial cell comprise E. coli, K. pneumoniae, R. gnavus, E. gallinarum, E. faecalis, E. faecium, or B. fragilis.
88. The method of any one of embodiments 81-87, wherein the bacteriophage is replication competent.
89. The method of any one of embodiments 81-87, wherein the bacteriophage is replication incompetent.
90. The method of any one of embodiments 81-89, wherein the anti-inflammatory peptide comprises pancreatitis-associated protein, Mycobacterium leprae Hsp65, bioactive heme oxygenase 1, EDSGTT, or TNFRI peptide.
91. A method of treating a disease or disorder in a subject comprising:
   (a) administering a recombinant lytic bacteriophage configured to infect a target bacterial cell within a subject, wherein the bacteriophage comprises a nucleic acid, wherein the nucleic acid comprises a sequence encoding the anti-inflammatory peptide and an operable lytic gene sequence;
   (b) producing the anti-inflammatory peptide in a target bacterial cell in a population of bacterial cells;
   (c) lysing the target bacterial cell,
   wherein the lysis is regulated by the operable lytic gene sequence;
   wherein the population of bacterial cells fluctuate between upper and lower limits
   where the lower limit does not equal zero CFUs and there is no upper limit.
92. The method of embodiment 91, wherein the anti-inflammatory peptide is soluble and is not expressed on a capsid protein of the recombinant lytic bacteriophage comprising the operable lytic gene sequence.
93. The method of embodiment 91 or 92, wherein the operable lytic gene sequence is derived from a bacteriophage sequence.
94. The method of any one of embodiments 91-93, wherein the operable lytic gene sequence regulates the lytic cycle of the bacteriophage.
95. The method of any one of embodiments 91-94, wherein the operable lytic gene sequence regulates the production of holin.
96. The method of embodiments 91-95, wherein the target bacterial cell comprises a bacterial species that bloom during a flare.
97. The method of embodiment 96, wherein the target bacterial cell comprises E. coli, K. pneumoniae, R. gnavus, E. gallinarum, E. faecalis, E. faecium, or B. fragilis.
98. The method of any one of embodiments 91-97, wherein the recombinant lytic bacteriophage is replication competent.
99. The method of any one of embodiments 91-97, wherein the recombinant lytic bacteriophage is replication incompetent.
100. The method of any one of embodiments 91-99, wherein the anti-inflammatory peptide comprises pancreatitis-associated protein, Mycobacterium leprae Hsp65, bioactive heme oxygenase 1, EDSGTT, or TNFRI peptide.
101. The method of embodiment 91, wherein the disease or disorder comprises Inflammatory Bowel Disease, Ulcerative Colitis, Crohn's Disease, Rheumatoid Arthritis, Psoriasis, Psoriatic Arthritis, Systemic Lupus Erythematosus, Lupus Nephritis, or cancer.

### Certain Terminology

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various bacteriophage and their components due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, +0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

“Complement” as used herein means 100% complementarity or identity with the comparator nucleotide sequence or it means less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refers to two or more sequences or subsequences that have at least about 70%, at least about 75%, at least about 80%, at least about 85%, at least about 90%, at least about 95, 96, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, CA). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection, disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50%, or more.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or on a subject. In some embodiments, the bacterial population comprises one or more target bacterial species. In some embodiments, the one or more bacteria species in the bacterial population comprise one or more strains of one or more bacteria. In some embodiments, the target bacterial population causes an “infection”, “a disease”, or “a condition” that is acute or chronic. In some embodiments, the target bacterial population causes an “infection”, “a disease”, or “a condition” that is localized or systemic. In some embodiments, the target bacterial population causes an “infection”, “a disease”, or “a condition” that is idiopathic. In some embodiments, the target bacterial population causes an “infection”, “a disease”, or “a condition” that is acquired through means, including but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias.

The terms “individual”, or “subject” as used herein includes any animal that has or is susceptible to an infection, disease or condition involving bacteria. Thus, in some embodiments, subjects are mammals, avians, reptiles, amphibians, fish, crustaceans, or mollusks. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). Fish subjects include but are not limited to species used in aquaculture (e.g., tuna, salmon, tilapia, catfish, carp, trout, cod, bass, perch, snapper, and the like). Crustacean subjects include but are not limited to species used in aquaculture (e.g., shrimp, prawn, lobster, crayfish, crab and the like). Mollusk subjects include but are not limited to species used in aquaculture (e.g., abalone, mussel, oyster, clams, scallop and the like). In some embodiments, suitable subjects include both males and females and subjects of any age, including embryonic (e.g., in-utero or in-ovo), infant, juvenile, adolescent, adult and geriatric subjects. In some embodiments, a subject is a human.

As used here the term “isolated” in context of a nucleic acid sequence is a nucleic acid sequence that exists apart from its native environment.

As used herein, “expression cassette” means a recombinant nucleic acid molecule comprising a nucleotide sequence of interest (e.g., the recombinant nucleic acid molecules disclosed herein), wherein the nucleotide sequence is operably associated with at least a control sequence (e.g., a promoter).

As used herein, “chimeric” refers to a nucleic acid molecule or a polypeptide in which at least two components are derived from different sources (e.g., different organisms, different coding regions).

As used herein, “selectable marker” means a nucleotide sequence that when expressed imparts a distinct phenotype to the host cell expressing the marker and thus allows such transformed cells to be distinguished from those that do not have the marker.

As used herein, “vector” refers to a composition for transferring, delivering or introducing a nucleic acid (or nucleic acids) into a cell.

As used herein, “pharmaceutically acceptable” means a material that is not biologically or otherwise undesirable, i.e., the material are administered to a subject without causing any undesirable biological effects such as toxicity.

As used herein the term “biofilm” means an accumulation of microorganisms embedded in a matrix of polysaccharide. Biofilms form on solid biological or non-biological surfaces and are medically important, accounting for over 80 percent of microbial infections in the body.

As used herein, the term “in vivo” is used to describe an event that takes place in a subject's body.

As used herein, the term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term antibody will be understood to include proteins having the characteristic two-armed, Y-shape of a typical antibody molecule as well as one or more fragments of an antibody that retain the ability to specifically bind to an antigen. Exemplary antibodies include, but are not limited to, a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CH1 domains), a F(ab′)2 fragment (including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for a scaffold, wherein the scaffold is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for a scaffold, wherein the scaffold is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2) or subclass.

### Examples

The following examples are included for illustrative purposes only and are not intended to limit the scope of the invention.

### Example 1: Example Engineered Phage Comprising an IL-10 Construct

Bacteriophage is engineered to comprise an IL-10 construct, serving as delivery vehicles for this agent. After phage engineering, the bacteriophages are recovered, mixed with the wild-type bacterial host, and plated. The plates are then checked for the presence of phage plaques. Individual plaques, indicative of bacterial cell lysis, are then screened by PCR for the correctly mutated phage genome.

Bacteriophage are engineered to contain antiholins, modified holins, and/or modified lysins. After phage engineering, the bacteriophages are recovered, mixed with the wild-type bacterial host, and plated. The plates are then checked for the presence of phage plaques. Individual plaques, indicative of bacterial cell lysis, are then screened by PCR for the correctly mutated phage genome.

### Example 2: Expression of Therapeutic Peptide from Engineered Phage Used in this Application

Expression of IL-10 in bacteria infected with engineered lytic phages is evaluated as compared to bacteria infected with non-engineered lytic phage variants. IL-10 expression from the phage genome is evaluated by ELISA (a). Equivalent amounts of phage particles (as determined by viral titration) are incubated in bacteria. The IL-10 concentration in the bacterial lysate is determined by placing the lysate on IL-10 specific mouse monoclonal antibody plates. The detection antibody is a biotinylated goat polyclonal antibody specific for IL-10. Parallel production and ELISA evaluation of each variant of phage are independently repeated three times to assess reproducibility.

### Example 3: Stability of Phage Engineered with Therapeutic Peptide Used in this Application

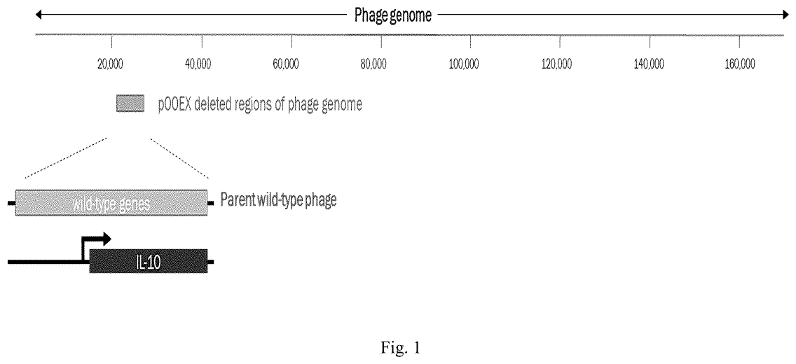

FIG. 1 depicts a schematic of non-engineered wild type (wt) phage genome as compared to engineered variants carrying the IL-10 therapeutic peptide construct within the p00EX deleted regions of the phage genome. Phage carrying the IL-10 therapeutic peptide construct is serially passaged to assess the stability of the inserts contained in the phage genome, as compared to wt. Wt and IL-10 phage is serially amplified on E. coli. Amplifications one through eight are performed as one step amplifications where 50 uL of a bacterial overnight culture is added to 5 mL of LB in a 15 mL falcon tube followed by the immediate addition of 1 μL of the previous lysate. The mixtures are grown for 10-16 hours at 37° C. in a shaking incubator. Following incubation, phage-bacterial mixtures are centrifuged for 10 min at 5,000 rcf and the supernatants are filtered through 0.45 μm syringe filters and stored at 4° C. For amplification nine, serial ten-fold dilutions of amplification eight are spotted onto soft agar overlays of strains. A single plaque from each plate is picked with a pipette into 200 μL of PBS to obtain amplification nine. Ten microliters of amplification nine, are added to 50 μL of bacteria overnight and 5 mL of LB, then grown for ˜16 hours followed by centrifugation and filtration. For sanger sequencing, phage DNA is amplified by PCR from lysates using primers flanking the engineering site. Sanger and NGS sequencing confirmed stability and integrity of the therapeutic peptide construct when loaded onto the phage genome.

### Example 4: Amplification of Phage Engineered with Therapeutic Peptide Construct Used in this Application

Wt and IL-10/therapeutic peptide phage are mixed with an exponentially growing culture of b1019 at an MOI of 1. At 0 min, 15 min, 30 min, 1 h, 2 h, 4 h, 7 h 10 min, and 24h after infection, samples are collected for PFU enumeration and RNA isolation and quantification. For PFU enumeration, the samples collected at each time point are filtered through 0.45 μm filters to separate the phage from the host bacteria. A soft agar overlay is prepared as described for Example 3. 10-fold serial dilutions of the phage samples are spotted onto the overlay and incubated at 37° C. The following day, plaques are counted and used to calculate the PFU/mL in the initial sample. IL-10 phage is able to amplify to essentially the same titer as wt in the host strain.

Wt and IL-10 phage are diluted to a particle count of 1e6, and each individual phage is used to infect a panel of 34 different bacteria at an MOI of 0.01. Optical Density (OD) readings at a wavelength of 600 nm are captured every hour over a 20-hour time course. The resulting OD readings are used to generate bacterial growth curves in the presence of one of the three phages or the control lacking any phage. Integration is used to calculate the Area Under the Curve (AUC) for each growth curve, where a smaller AUC upon phage addition indicates reduced bacterial load. The AUC Ratio, in which the AUC calculation of strain growth in the presence of phages is divided by the AUC of strain growth in the absence of phage is calculated.

### Example 5: Phage Lytic Activity when Engineered with Therapeutic Peptide Construct

Top agar overlays are prepared by mixing 100 μL of a saturated overnight culture of the indicator strain with 6 mL of 0.375% agar in LB containing 10 mM MgCl₂ and 10 mM CaCl₂). After the top agar solidified, 2 μL drops of serial 10-fold dilution series of wt and IL-10 are spotted onto the surface of the top agar. Plates are incubated at 37° C. for ˜18h, then imaged using a Keyence BZ-X800 microscope at 4× and 10× magnification. Phage engineered with IL-10 retains its lytic ability as evidenced by the ability to form plaques of similar morphology.

### Example 6: Increased Latent Period of an Engineered Phage

Measurement of bacteriophage lysis kinetics of phage modified to increase the time to lysis

The wildtype and lysis-delayed phage strains synchronously infect the target E. coli bacterial strain. At 5-minute intervals, the sample is plaqued onto an overlay of E. coli bacteria. The next day, the number of plaques from the wildtype and lysis impaired phage are counted. In this assay, phage lysis is inhibited if the mutant demonstrate reduced PFUs at earlier time points.

ELISA of IL-10 expression from holin-modified phage vs non holin-modified phage

The holin-modified bacteriophage invade the bacterial host, and cause inactivation of the bacterial host prior to production of a detectable or significant number of IL-10, measured by ELISA as previously described.

### Example 7: Continued Expression of IL-10

Following the bacteriophage's injection of the IL-10 gene protein construct into a bacteria, IL-10 expression within the bacteria infected by the bacteriophage is measured by quantifying the level of mRNA via qPCR or RNA-seq. The stability of IL-10 expression in prokaryotic cells throughout the population is measured 12 hours, 24 hours, 36 hours, 48 hours, 72 hours, and 96 hours after bacteriophage delivery of the IL-10 construct. Over this same period of time both bacterial colony forming units (CFUs) and phage plaque forming units (PFUs) are measured as previously described.

### Example 8: Example Engineered Phage Comprising a Nanobody

Bacteriophage is engineered to comprise a sequence encoding a nanobody targeting Tumor Necrosis Factor alpha (TNF). After electroporation, the bacterial cells are recovered, mixed with the wild-type bacterial host, and plated. The plates are then checked for the presence of phage plaques. Individual plaques, indicative of bacterial cell lysis, are then screened by PCR for the correctly mutated phage genome.

Bacteriophage are engineered to contain antiholins, modified holins, and/or modified lysins. After phage engineering, the bacteriophages are recovered, mixed with the wild-type bacterial host, and plated. The plates are then checked for the presence of phage plaques. Individual plaques, indicative of bacterial cell lysis, are then screened by PCR for the correctly mutated phage genome.

### Example 9: Expression of Nanobody from Engineered Phage Used in this Application

Expression of TNF nanobody in bacteria infected with engineered lytic phages is evaluated as compared to bacteria infected with non-engineered lytic phage variants. TNF nanobody expression from the phage genome is evaluated by ELISA. Equivalent amounts of phage particles (as determined by viral titration) are incubated in bacteria. The TNF nanobody concentration in the bacterial lysate is determined by placing the lysate on TNF nanobody specific mouse monoclonal antibody plates. The detection antibody is a biotinylated goat polyclonal antibody specific for TNF nanobody. Parallel production and ELISA evaluation of each variant of phage are independently repeated three times to assess reproducibility.

### Example 10: Stability of Phage Engineered with Nanobody Used in this Application

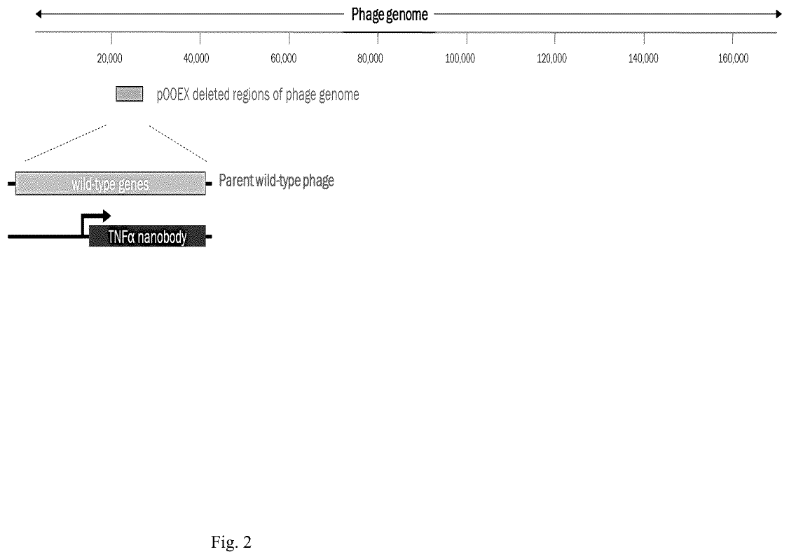

FIG. 2 depicts a schematic of non-engineered wild type (wt) phage genome as compared to engineered variants carrying the Tumor necrosis factor alpha (TNFα) nanobody construct within the p00EX deleted regions of the phage genome.

Phage carrying the nanobody construct is serially passaged to assess the stability of the inserts contained in the phage genome, as compared to wt. Wt and nanobody phage is serially amplified on E. coli. Amplifications one through eight are performed as one step amplifications where 50 uL of a bacterial overnight culture is added to 5 mL of LB in a 15 mL falcon tube followed by the immediate addition of 1 μL of the previous lysate. The mixtures are grown for 10-16 hours at 37° C. in a shaking incubator. Following incubation, phage-bacterial mixtures are centrifuged for 10 min at 5,000 rcf and the supernatants are filtered through 0.45 μm syringe filters and stored at 4° C. For amplification nine, serial ten-fold dilutions of amplification eight are spotted onto soft agar overlays of strain b1121 or b1126. A single plaque from each plate is picked with a pipette into 200 μL of PBS to obtain amplification nine. Ten microliters of amplification nine, are added to 50 μL of bacteria overnight and 5 mL of LB, then grown for ˜16 hours followed by centrifugation and filtration. For sanger sequencing, phage DNA is amplified by PCR from lysates using primers flanking the engineering site. Sanger and NGS sequencing confirmed stability and integrity of the nanobody construct when loaded onto the phage genome.

### Example 11: Amplification of Phage Engineered with Nanobody Used in this Application

Wt and TNF nanobody phage are mixed with an exponentially growing culture of b1019 at an MOI of 1. At 0 min, 15 min, 30 min, 1 h, 2 h, 4 h, 7 h 10 min, and 24h after infection, samples are collected for PFU enumeration and RNA isolation and quantification. For PFU enumeration, the samples collected at each time point are filtered through 0.45 μm filters to separate the phage from the host bacteria. A soft agar overlay is prepared as described in Example 3. 10-fold serial dilutions of the phage samples are spotted onto the overlay and incubated at 37° C. The following day, plaques are counted and used to calculate the PFU/mL in the initial sample. TNF nanobody phage is able to amplify to essentially the same titer as wt in the host strain.

Wt and nanobody phage are diluted to a particle count of 1e6, and each individual phage is used to infect a panel of 34 different bacteria at an MOI of 0.01. Optical Density (OD) readings at a wavelength of 600 nm are captured every hour over a 20-hour time course. The resulting OD readings are used to generate bacterial growth curves in the presence of one of the three phages or the control lacking any phage. Integration is used to calculate the Area Under the Curve (AUC) for each growth curve, where a smaller AUC upon phage addition indicates reduced bacterial load. The AUC Ratio, in which the AUC calculation of strain growth in the presence of phages is divided by the AUC of strain growth in the absence of phage is calculated.

### Example 12: Phage Lytic Activity when Engineered with Nanobody Construct

Top agar overlays are prepared by mixing 100 μL of a saturated overnight culture of the indicator strain with 6 mL of 0.375% agar in LB containing 10 mM MgCl₂ and 10 mM CaCl₂). After the top agar solidified, 2 μL drops of serial 10-fold dilution series of wt and nanobody are spotted onto the surface of the top agar. Plates are incubated at 37° C. for ˜18h, then imaged using a Keyence BZ-X800 microscope at 4× and 10× magnification. Phage engineered with TNF nanobody retains its lytic ability as evidenced by the ability to form plaques of similar morphology.

### Example 13: Increased Latent Period of an Engineered Phage

Measurement of Bacteriophage Lysis Kinetics of Phage Modified to Increase the Time to Lysis

The wildtype and lysis-delayed phage strains synchronously infect the target E. coli bacterial strain. At 5-minute intervals, the sample is plaqued onto an overlay of E. coli bacteria. The next day, the number of plaques from the wildtype and lysis impaired phage are counted. In this assay, phage lysis is inhibited if the mutant demonstrates reduced PFUs at earlier time points.

ELISA of TNF nanobody expression from holin-modified phage vs non holin-modified phage

The holin-modified bacteriophage invade the bacterial host, and cause inactivation of the bacterial host prior to production of a detectable or significant number of TNF nanobody, measured by ELISA as previously described.

### Example 14: Continued Expression of TNF Nanobody

Following the bacteriophage's injection of the TNF nanobody gene protein construct into a bacteria, TNF nanobody expression within the bacteria infected by the bacteriophage is measured by quantifying the level of mRNA via qPCR or RNA-seq. The stability of TNF nanobody expression in prokaryotic cells throughout the population is measured 12 hours, 24 hours, 36 hours, 48 hours, 72 hours, and 96 hours after bacteriophage delivery of the TNF nanobody construct. Over this same period of time both bacterial colony forming units (CFUs) and phage plaque forming units (PFUs) will be measures as previously described.

### Example 15: Example Engineered Phage Comprising an Anti-Inflammatory Peptide

Bacteriophage is engineered to comprise a sequence encoding a TNFα Receptor peptide (TNFRI). After electroporation, the bacterial cells are recovered, mixed with the wild-type bacterial host, and plated. The plates are then checked for the presence of phage plaques. Individual plaques, indicative of bacterial cell lysis, are then screened by PCR for the correctly mutated phage genome.

Bacteriophage are engineered to contain antiholins, modified holins, and/or modified lysins. After phage engineering, the bacteriophages are recovered, mixed with the wild-type bacterial host, and plated. The plates are then checked for the presence of phage plaques. Individual plaques, indicative of bacterial cell lysis, are then screened by PCR for the correctly mutated phage genome.

### Example 16: Expression of Anti-Inflammatory Peptide from Engineered Phage Used in this Application

Expression of TNFRI in bacteria infected with engineered lytic phages is evaluated as compared to bacteria infected with non-engineered lytic phage variants. TNFRI expression from the phage genome is evaluated by ELISA (a). Equivalent amounts of phage particles (as determined by viral titration) are incubated in bacteria. The TNFRI concentration in the bacterial lysate is determined by placing the lysate on TNFRI specific mouse monoclonal antibody plates. The detection antibody is a biotinylated goat polyclonal antibody specific for TNFRI. Parallel production and ELISA evaluation of each variant of phage are independently repeated three times to assess reproducibility.

### Example 17: Stability of Phage Engineered with Anti-Inflammatory Peptide Used in this Application

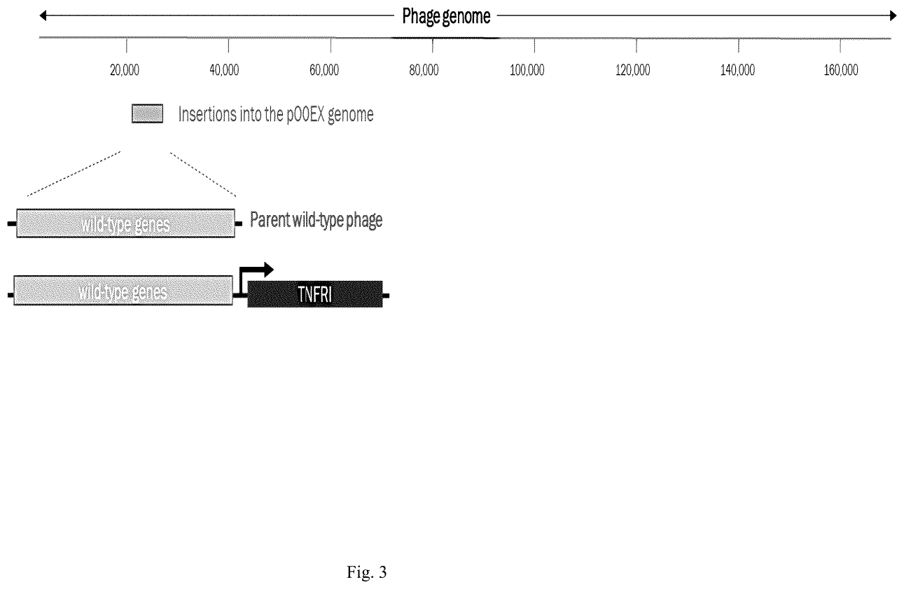

FIG. 3 depicts a schematic of non-engineered wild type (wt) phage genome as compared to engineered variants carrying the TNFα Receptor peptide (TNFRI) peptide construct into the p00EX region of the phage genome.

Phage carrying the anti-inflammatory peptide construct is serially passaged to assess the stability of the inserts contained in the phage genome, as compared to wt. Wt and anti-inflammatory peptide phage is serially amplified on E. coli. Amplifications one through eight are performed as one step amplifications where 50 uL of a bacterial overnight culture is added to 5 mL of LB in a 15 mL falcon tube followed by the immediate addition of 1 μL of the previous lysate. The mixtures are grown for 10-16 hours at 37° C. in a shaking incubator. Following incubation, phage-bacterial mixtures are centrifuged for 10 min at 5,000 rcf and the supernatants are filtered through 0.45 μm syringe filters and stored at 4° C. For amplification nine, serial ten-fold dilutions of amplification eight are spotted onto soft agar overlays of strains. A single plaque from each plate is picked with a pipette into 200 μL of PBS to obtain amplification nine. Ten microliters of amplification nine, are added to 50 μL of bacteria overnight and 5 mL of LB, then grown for ˜16 hours followed by centrifugation and filtration. For sanger sequencing, phage DNA is amplified by PCR from lysates using primers flanking the engineering site. Sanger and NGS sequencing confirmed stability and integrity of the anti-inflammatory peptide construct when loaded onto the phage genome.

### Example 18: Amplification of Phage Engineered with Anti-Inflammatory Peptide Construct Used in this Application

Wt and TNFRI phage are mixed with an exponentially growing culture of b1019 at an MOI of 1. At 0 min, 15 min, 30 min, 1 h, 2 h, 4 h, 7 h 10 min, and 24h after infection, samples are collected for PFU enumeration and RNA isolation and quantification. For PFU enumeration, the samples collected at each time point are filtered through 0.45 μm filters to separate the phage from the host bacteria. A soft agar overlay is prepared as described for Example 3. 10-fold serial dilutions of the phage samples are spotted onto the overlay and incubated at 37° C. The following day, plaques are counted and used to calculate the PFU/mL in the initial sample. TNFRI phage is able to amplify to essentially the same titer as wt in the host strain.

Wt and anti-inflammatory peptide phage are diluted to a particle count of 1e6, and each individual phage is used to infect a panel of 34 different bacteria at an MOI of 0.01. Optical Density (OD) readings at a wavelength of 600 nm are captured every hour over a 20-hour time course. The resulting OD readings are used to generate bacterial growth curves in the presence of one of the three phages or the control lacking any phage. Integration is used to calculate the Area Under the Curve (AUC) for each growth curve, where a smaller AUC upon phage addition indicates reduced bacterial load. The AUC Ratio, in which the AUC calculation of strain growth in the presence of phages is divided by the AUC of strain growth in the absence of phage is calculated.

### Example 19: Pha2e Lytic Activity when Engineered with Anti-Inflammatory Peptide Construct

Top agar overlays are prepared by mixing 100 μL of a saturated overnight culture of the indicator strain b1121 with 6 mL of 0.375% agar in LB containing 10 mM MgCl₂ and 10 mM CaCl₂). After the top agar solidified, 2 μL drops of serial 10-fold dilution series of wt and anti-inflammatory peptide are spotted onto the surface of the top agar. Plates are incubated at 37° C. for ˜18h, then imaged using a Keyence BZ-X800 microscope at 4× and 10× magnification. Phage engineered with TNFRI retains its lytic ability as evidenced by the ability to form plaques of similar morphology.

### Example 20: Increased Latent Period of an Engineered Phage

Measurement of bacteriophage lysis kinetics of phage modified to increase the time to lysis

The wildtype and lysis-delayed phage strains synchronously infect the target E. coli bacterial strain. At 5-minute intervals, the sample is plaqued onto an overlay of E. coli bacteria. The next day, the number of plaques from the wildtype and lysis impaired phage are counted. In this assay, phage lysis is inhibited if the mutant demonstrates reduced PFUs at earlier time points.

ELISA of TNFRI Expression from Holin-Modified Phage Vs Non Holin-Modified Phage

The holin-modified bacteriophage invade the bacterial host, and cause inactivation of the bacterial host prior to production of a detectable or significant number of TNFRI, measured by ELISA as previously described.

### Example 21: Continued Expression of TNFRI

Following the bacteriophage's injection of the TNFRI gene protein construct into a bacteria, TNFRI expression within the bacteria infected by the bacteriophage is measured by quantifying the level of mRNA via qPCR or RNA-seq. The stability of TNFRI expression in prokaryotic cells throughout the population is measured 12 hours, 24 hours, 36 hours, 48 hours, 72 hours, and 96 hours after bacteriophage delivery of the TNFRI construct. Over this same period of time both bacterial colony forming units (CFUs) and phage plaque forming units (PFUs) will be measures as previously described.

### Example 22: Tunable Delivery of a Recombinant Protein

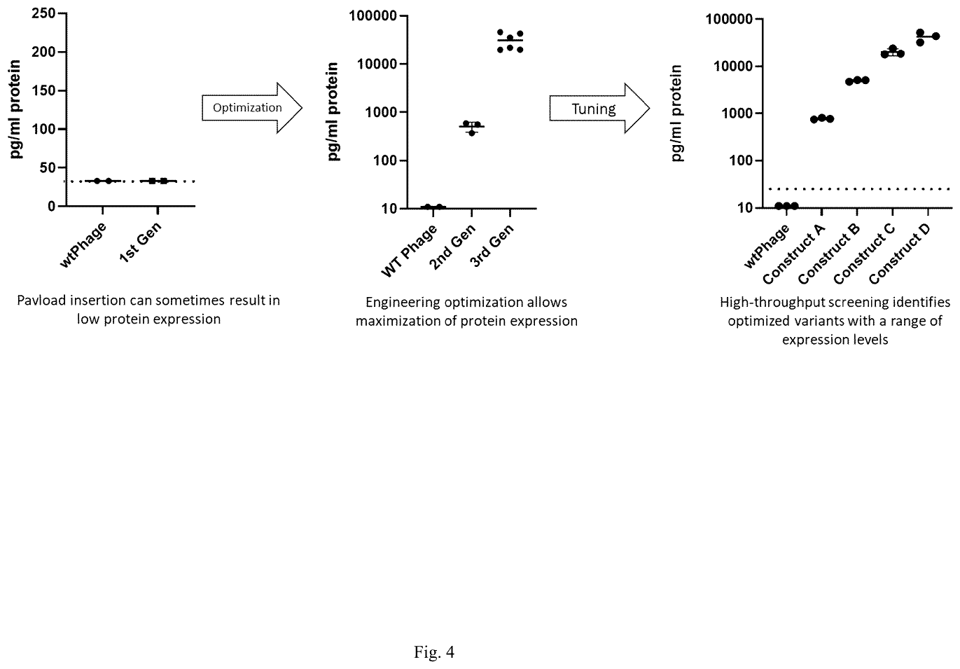

The E. coli-targeting bacteriophage p00ex was engineered to contain mIL-10 (SEQ ID NO: 83) at different sites and with expression driven by different promoters (see example sequences below). mIL-10 expression was measured by flow-cytometry based ELISA. The results are shown in FIG. 4: the left panel shows measured protein expression from the unengineered wild type phage (‘wtPhage’) and the first phage engineered with an mIL-10 payload. In both cases the level of protein expression is below the limit of detection of the assay. The center panel shows mIL-10 SEQ ID NO: 84) expression from the wild type phage and two optimized engineered phages, with the mIL-10 payload being driven by a different promoter and in a different insertion site. The right panel shows mIL-10 expression from the wild type phage and a panel of engineered variants tuned to provide different levels of protein expression.

The following is the key for FIG. 4: p00ex is indexed such that the first base of the YP_009153762.1 terminase small subunit is base 1. 1st gen=one copy of mIL-10 (SEQ ID NO: 67, encoding SEQ ID NO: 68) driven by Pweak (SEQ ID NO: 12) in the del 1 site (reverse orientation removing base pairs 142,546-144,750); 2nd gen=1st gen plus a second copy of mIL-10 (SEQ ID NO: 67) driven by the recombinase promoter (SEQ ID NO: 13) in the late sigma site (reverse orientation after base pair 112,290); 3rd gen=2nd gen plus a third copy of mIL-10 driven by the rIII promoter (SEQ ID NO: 14) in the rIII site (reverse orientation after base pair 35,142); Construct A=one copy of mIL-10 driven by the gyrase promoter; Construct B=one copy of mIL-10 (SEQ ID NO: 67) driven by the recombinase promoter; Construct C=one copy of mL-10 (SEQ ID NO: 67) driven by the protector from prophage promoter; and Construct D=one copy of mL-10 driven by the rIII promoter. Additional engineered phage are listed in Tables 1A-1B

TABLE 1A
mIL-10 phages

| Phage | mIL-10 | Deletion/inser- | Deletion/inser- | |
|---|---|---|---|---|
| ID | copies | tion? | tion site(s) | Promoter |
| p00exe043 | 1 | deletion | del1 | Pweak |
| p00exe044 | 1 | deletion | del1 | Pmid |
| p00exe045 | 1 | deletion | del1 | pStrong |
| p00exe046 | 1 | deletion | del5 | pStrong |
| p00exe070 | 1 | deletion | del1 | DNA pol |
| p00exe071 | 1 | deletion | del1 | gp55 |
| p00exe072 | 1 | deletion | del1 | recombinase |
| p00exe073 | 2 | deletion + | del1/gp55 | Pweak/late |
| | | insertion | | sigma |
| p00exe074 | 1 | insertion | late sigma | YP |
| p00exe075 | 2 | deletion | del1/late sigma | Pweak/gp55 |

| | SEQ | |
|---|---|---|
| | ID | |
| Site | NO: | DNA sequence (5′-3′) |
| del1 | 72 | CTTAGCCCCGACCGAAAGGT |
| | | TGGGGCTTTTTGGTATAAAT |
| | | ATTAGTATATTAAATCTACA |
| | | AATTAAAACAGGAAATAAGA |
| | | TGAAATCATATACTCAATTT |
| | | TTAAATGAAGCGGTGTTAAA |
| | | TGAAGCATCTAGCACCGAAA |
| | | TTCAAGCTGTTGCAAAAGCT |
| | | GCCATTGCCGCGGGTAAATA |
| | | TTCCTATAAAGATGCTTCTG |
| | | ATGAATCGCGATTCCAGTTT |
| | | GCACGCGACATGAAAGCGGA |
| | | AGGATTTACGGGAAATGCAG |
| | | TTAGTATGGCCTGGAAAAGT |
| | | TTAGTTGCTACTGGCGCTGC |
| | | TTTTGCAAAGGCTTCGGGTA |
| | | AACCTGCTCCTAAAGCAGAT |
| | | CCTAAAGCGGCACAAGAAAA |
| | | AAAATATCGTTAAAGGAATT |
| | | ATCGCTAAATATGAAGCTAT |
| de15 | 73 | CTATAGACTAAAGGTCACA |
| Pweak | 74 | TTTACAGCTAGCTCAGTCCT |
| | | AGGGACTGTGCTAGC |
| Pmid | 75 | TTTACGGCTAGCTCAGTCCT |
| | | AGGTACAATGCTAGC |
| pStrong | 76 | TTGACGGCTAGCTCAGTCCT |
| | | AGGTACAGTGCTAGC |
| DNA | 77 | CCGACAAATAGTACTAATTT |
| pol | | AAGCAAGGGGCTTCGGCCCC |
| | | TTAATTGGAGTATAATATAT |
| | | CAAGAGCCTAATAACTCGGG |
| | | CTATAAACTAAGGAATATCT |
| gp55 | 78 | GTTGATAAAGAAATTAAAAA |
| | | GGGACAGTATTATCTCATTA |
| | | ATGGTAATGCTGTTCGTGTT |
| | | ACTTACGTAAATGGTTATGA |
| | | TGTTTATTATCTTATACTTA |
| | | AGTTACATAAACACATGATT |
| | | TGCGATCGTGCTGCATTTAG |
| | | TTCAGTTGCTAAGGAAATTA |
| | | AACTCC |
| recomb | 79 | GCACAAGGCGTTGTTTGCAT |
| inase | | |
| | | GGCTGAATTTGATGAATACT |
| | | TTTTAGATTATGATGATATG |
| | | ATAGAATGGTCTAAAAGATA |
| | | CATTAAAAGGAATCTTTTGT |
| | | GAAGAATTTTAAACTAAACC |
| | | GAGTTAGGTATCAAAATATA |
| late | 80 | AGCATACTGTTTACTTAGAT |
| sigma | | |
| | | AACTGATATCCTCTATGCTT |
| | | TAAGATAGATCTTCAAATAT |
| | | TATGATATAATAGATCTATG |
| | | AATTGAGCTAAGAGGTGAAA |
| YP | 81 | GTCGCAATGATGTACGAAGG |
| | | CTGGAAGGGTGCCAAAAAGT |
| | | TTAGCTAAGGGCTTCGGCCC |
| | | TTTTTGGATAATAAAATTTT |
| | | AACGTAATTGAGGATAATGT |
| RBS-A | 82 | ATTAAAGAGGAGAAA |

### Example 23: Protein Delivery Using Generalist Promoters

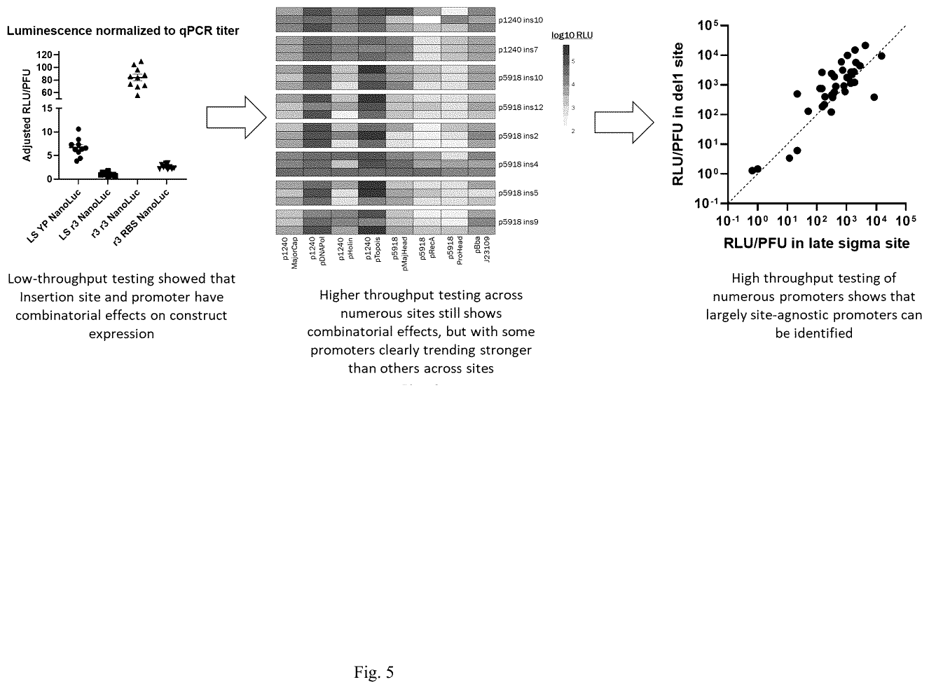

Various bacteriophages were engineered to contain nanoLuciferase (“nLuc”, SEQ ID NO: 69, 70) at different sites and with expression driven by different promoters and luminescence (RLU) per phage (PFU) was measured as a reporter of protein expression level. Results are shown in FIG. 5.

The left panel of FIG. 5 shows several engineered variants of the E. coli-targeting phage p00ex (Tequatrovirus, ATCC deposit number PTA-127145, deposited with the ATCC Oct. 21, 2021). Labels on the plot indicate first the insertion site (“LS”=late sigma transcription factor site; “r3”=rIII anti-holin site) and second the promoter driving nLuc (“YP”=YP [unknown putative ORF] promoter (SEQ ID NO: 15), “r3”=rIII anti-holin promoter, (SEQ ID NO: 14) “RBS”=no promoter present but does have a ribosome binding site) (SEQ ID NO: 71. By comparing variants with nLuc in the same site but with different promoters, or with the same promoter in different sites, one can see that both site and promoter play and important role in expression level.

The center panel shows several engineered variants of the K. pneumoniae-targeting phages p1240 and p5918. The x-axis shows different promoters derived from one of the two phages (indicated by “p1240” or “p5918”) or a synthetic promoter derived from E. coli (pBba J23109). The Y-axis shows different insertion sites in each of the two phages. The color of each box indicates the level of expression. Both promoter identity and insertion site contribute to determining expression level, but some promoters can be seen to be generally stronger than others regardless of insertion site. p1240 is a Slopekvirus; p5918 is a Marfavirus.

The right panel again shows engineered variants of the E. coli-targeting phage p00ex. The phage was engineered at two different insertion sites with nLuc expression driven by 45 different promoters. Each dot represents a single promoter. Given that most of the dots cluster close to the 1:1 line (i.e., where expression level is identical between the two insertion sites), we can conclude that promoter is the primary driver of expression level.

Table 2: Additional promoter sequences

TABLE 2
Additional promoter sequences

| | promoter | | |
|---|---|---|---|
| SEQ | number | | |
| ID | (registered | | Promoter Sequence |
| NO | as this) | promoter name | Used |
| 23 | prom08 | Cascade | AGCAGCCCTGCGCGAGCTGGAGTTCGCCGA |
| | | endogenous_ | CGGCTGCGCCGCCATCGCCCGTCGCCTGCA |
| | | promoter | GCCATATCTGGTGCAGATGCCACGCAAGGG |
| | | | TTATCAGGCATTGCGGGAAGCCGGTGCGAT |
| | | | CCAGGCGGCGGCAGGTACGCGTTATGGTGA |
| | | | GCAGTTTATGGCGTTGGTCAACCCTGATCT |
| | | | GTATCACCACCAATTCGGGTTGCACTGGGA |
| | | | TAATCCGGCCTTTGTCAGCAGCGAGCGGCT |
| | | | ATGTTGGTAGTCGGGACGCGCAACAGCGGC |
| | | | CTGGCCTGGATGATGTGAAAGGGAGGGCCG |
| 24 | prom09 | pWeak_ | TTTACAGCTAGCTCAGTCCTAGGGACTGTG |
| | | promoter | CTAGCATTAAAGAGGAGAAA |
| 25 | prom10 | rnnB_ | GAAAATTATTTTAAATTTCCTCTAGTCAGG |
| | | promoter | CCGGAATAACTCCCTATAATGCGACACCA |
| 26 | prom11 | YP_ | ATTTACGATATTAATGTATCACGAGCTCCG |
| | | 001595208.1_ | TCAATGGTTACTATTCCAGCCGAAGAACTA |
| | | gp49.1_ | GATCGTCTTCAGAAAATTGAAGAGCTTCTT |
| | | conserved_ | TGGGAAATTGAATCTGATTTGCCATCAGGA |
| | | protein. | TTAGAATCCTGGATTGATT |
| | | Escherichia_ | |
| | | phage_ | |
| | | JS98._ | |
| | | promoter | |
| 27 | prom12 | YP_ | AAATATAGCATTGATGATGCTTTTAATTAT |
| | | 003734327.1_ | GAAGAAGAATTCGAAACGGAAATTCAATTC |
| | | uvsY_.._2_ | TTAATGAAAAAGTATAATCTCAAGCGTCAG |
| | | gene_ | GATATTCGTATCCTGGCCGACCACCCATGC |
| | | product_ | GGTGAAGATGTTCTTTATATTAAAGGAAAA |
| | | Enterobacteria_ | TTTGCCGGATATCTTGATGAATATTTTTAT |
| | | phageIME08._ | TCTAAAGATATGGGCATTGATATGCATATG |
| | | promoter | AGAGTTGTATAAATAGATATATAATTCAGA |
| | | | GGAGACAATC |
| 28 | prom13 | YP_ | AATAACATAGAAAAGATTTATCGTCTTTGT |
| | | 003734358.1_ | GATAAAATTGAAAAAGAAAAGAAATATCTA |
| | | cd.3_ | TTTTGTCTATGGCCTATTGTTGACGGAAGA |
| | | gene_ | GTAGGCCTAGATGTTCTTGATTATGAAACA |
| | | product_ | GAAGACAAAGTAGATGGCGCAACTTTTGAT |
| | | Enterobacteria_ | AACGCTTTGGATGTTATTGATTGGCTCGAA |
| | | phage_I | GAAAATTATGTGAGGTAAAT |
| | | ME08._ | |
| | | promoter | |
| 29 | prom14 | YP_ | AAAACATTTAAAATTGTCGTAGAATTTTAT |
| | | 003734391.1_ | AACGGCGAAGGTGTAGTCCATCTTAAAGCG |
| | | arn.3_ | GCCAACCAGTTTGATGCGGTAAGAGTATAT |
| | | gene_ | TGTCAATGCTTTGAAAGTTCTAAACAAGCT |
| | | product_._ | ATAAAAATTAAAAGTGTTGAGGAAACCAA |
| | | Enterobacteria_ | |
| | | phage_ | |
| | | IME08._ | |
| | | promoter | |
| 30 | prom15 | YP_ | GATTTATTTGAGATGTTAGAAGATAATCAT |
| | | 006986570.1_ | TCTACGAATAACCAGAATGATTCTAGTGAT |
| | | ADP. | TATAAGAAAGAGTACCGTATAGTATTACAG |
| | | ribosylase_ | AATTATGGAATTGAAGCCCCAGATGCTCTT |
| | | Escherichia_ | CTAGAAGAACTAGCTTCATACCATCTTGAC |
| | | phage_ | CCTCCGCCCTGGGCTCCCTGGGCAAAATAA |
| | | vB_ | TTCAAAAAGTTGTTTACTTTCCTTTCTAAC |
| | | EcoM_ | GATGATATGATAGCTTCTGAAGTATACGGG |
| | | ACG.C40._ | AGGCTATC |
| | | promoter | |
| 31 | prom16 | YP_ | TAATGATTTAGATTCTAGCGGAAATCCTTC |
| | | 006986711.1_ | ACACACTGCCGGTGGAACAGTTGGAACAAC |
| | | baseplate_ | GTCAGTAACGCTTGAAAATGCAAATCTTCC |
| | | wedge_ | TGCGACTAAAACTGACGAAAGAGTTTTAAT |
| | | completion_ | TGAAGATGAAAATGGATCAGTTATTATTGG |
| | | tail_ | AGGTTGTCAATATGACCCAGATGAAACTGG |
| | | pin_ | TCCTATATATACAAAATATCGTGAAGACTA |
| | | Escherichia_ | TGCAACAACAAACTCTTCACATACTCCTCC |
| | | phage_ | TACTAATATTAGTAATATTCAACCGTCTAT |
| | | vB_ | TACTGTATACCGTTGGATAAGGATTGCATA |
| | | EcoM_ | |
| | | ACG.C40._ | |
| | | promot | |
| 32 | prom17 | YP_ | GGAATGCATGTTGGTGGTGTTCAGGCACAA |
| | | 006986713.1_ | CAAATGTCATACCATAAACATGCTGGTGGT |
| | | fibritin_ | TGGGGTGAATATAACAGAAGTGAAGGCCCA |
| | | neck_ | TTTGGCGCGTCAGTTTATCAAGGATATCTT |
| | | whiskers_ | GGAACTAGAAAATATTCCGACTGGGATAAT |
| | | Escherichia_ | GCTTCATACTTCACTAATGATGGATTTGAA |
| | | phage_ | TTAGGTGGACCGAGAGATGCTCATGGTACA |
| | | vB_ | CTTAATCGTGAAGGATTAATTGGTTATGAA |
| | | EcoM_ | ACTAGACCATGGAATATATCATTAAACTAT |
| | | ACG.C40. | ATTATTAAAGTTCATTACTAAGGATTACAA |
| | | promoter | |
| 33 | prom18 | YP_ | AACTTACTTCACTTATAAATGGAAATAATC |
| | | 006986714.1_ | CTGACGGATCAACTGTTGAAGAACGAGGAT |
| | | neck_ | TAACTAATTCTATAAAAACGAATGAAACCA |
| | | protein_ | ACATTGCGGCAGTCACACATGAAGTAAATA |
| | | Escherichia_ | CAGCTAAAGACAATATATCCTCTTTACAGA |
| | | phage_ | GCAGCGTTCAAGCTCTACAAGAAGCAGGTT |
| | | vB_ | ATATTCCTGAAGCTCCAAAAGATGGCCAAG |
| | | EcoMACG.C40._ | CTTACGTTCGTAAAGACGGCGAATGGGTAC |
| | | promoter | TACTTTCTACCTTTTTATCACCAGCATAAC |
| | | | ATGGGGCCGCAAGGCCCCAAAGGATTTTAA |
| 34 | prom19 | YP_ | ATAGTTGGTCATTATCTGAACGATACAATA |
| | | 006986715.1_ | ATCCAGACCATAATTTAGTAGGTCGTGTTG |
| | | neck_ | TCGGTCAAGATCCAAATGTTAAGCAAGGTG |
| | | protein_ | CTTATAATAATCGTTGGGTGAAAGACTATG |
| | | Escherichia_ | CAACAGCTTTAGCTAAAGAATTGAATGGTC |
| | | phage_ | AAATTTTAGCGCGTCACCAGGGAATGATGC |
| | | vB_ | TTCCAGGCGGTGTTACGATTGATGGACAAC |
| | | EcoMACG.C40._ | GTTTAATAGAAGAAGCTCGATTAGAAAAAG |
| | | promoter | AAGCATTACGCGAAGAATTATACTTACTTG |
| | | | ACCCTCCATTTGGAATTTTTGTAGGTTAAT |
| 35 | prom20 | YP_ | AACAAATGCTAATGGACGCAGCCAAGATTT |
| | | 006986718.1_ | TTCTTGAGACGGCAAAGAATGCTGATTCTC |
| | | terminase_ | CTCGTCACATGGAAGTATTTGCAACTCTTA |
| | | DNA_ | TGGGGCAAATGACTACGACGAACAGAGAAA |
| | | packaging_ | TACTGAAGCTTCACAAAGACATGAAAGATA |
| | | enzyme_ | TTACATCTGAACAGGTTGGCACCAAAGGCG |
| | | large_ | CTGTTCCTACAGGTCAAATGAATATTCAGA |
| | | subunit_ | ATGCGACAGTATTCATGGGTTCACCAACAG |
| | | Escherichia_ | AATTAATGGACGAAATTGGTGATGCTTACG |
| | | phage_ | AGGCTCAAGAAGCTCGTGAGAAGGTGATAA |
| | | vB_ | |
| | | EcoM_ | |
| | | ACG.C40._ | |
| | | promoter | |
| 36 | prom21 | YP_ | AGATGCAGTTAAAAATAAGACGTTATTTGA |
| | | 006986749.1_ | GAAAATGACATCGAGTTTAACTAACGTTCT |
| | | baseplate_ | TGTAGTTTCAAACCCGACAATTTGGATGGT |
| | | tail_ | GAAAAACTTTGGTGCAACATCTAAGTTTGA |
| | | tube_ | TGGAAAAACGGAAATATTCGGTCCATGTCA |
| | | initiator_ | AATCCAGAGTATCAGATTTGATAAAACACC |
| | | Escherichia_ | TAATGGTAACTTTAACGGATTAGCTATTGC |
| | | phage_ | TCCAAATCTCCCTAGTACATTTACTCTCGA |
| | | vB_ | GATTACTATGAGAGAAATTATCACGTTAAA |
| | | EcoM_ | CCGTGCTTCTTTATATGCGGGGACTTTTTA |
| | | ACG.C40._ | |
| | | promoter | |
| 37 | prom22 | YP_ | TAAGGTGACTTATACTTGTAATCTATCTAA |
| | | 006986787.1_ | ACGGGGGACCTCTCTAGTAGACAATCCCGT |
| | | dTMP_ | GCTAAATTGTAGGACTACCCTTTAATAATT |
| | | thymidylate_ | TCATCAGGATTAGTTACTTACCGTGTAAAA |
| | | synthase_ | TCTGATAAATGGAATTGGTTCTACATAAAT |
| | | Escherichia_ | GCCTAACGACTATCCCTTTGGGGAGTAGGG |
| | | phage_ | TCAAGTGACTCGAAACGATAGACAACTTGC |
| | | vB_ | TTTAACAAGTTGAAGATATAGTCTGATCTG |
| | | EcoM_ | CATGGTGACATGCAGCTGGATATAATTCCG |
| | | ACG.C40._ | GGGTAAGATTAACGACCTTATCTGAACATA |
| | | promoter | |
| 38 | prom23 | YP_ | GTTTGGTAACACACTTGATTCGCTTTACCA |
| | | 006986804.1_ | AGATTGGATTACTTATCCAACGACCCCAGA |
| | | hinge_ | AGCACGTACCACTCGCTGGACACGTACATG |
| | | connector_ | GCAGAAAACCAAAAACTCTTGGTCAAGTTT |
| | | of_ | TGTTCAGGTATTTGACGGAGGTAACCCTCC |
| | | long_ | TCAACCTTCAGATATAGGAGCGATCCCATC |
| | | tail_ | TGATAATGGAATAATAGGTAATCTTACTAT |
| | | fiber_ | TCGTGATTTCTTACGAATTGGTAATGTTCG |
| | | proximal_ | CATTATTCCTGACCCAGTGAATAAAACTGT |
| | | connector_ | TAAATTTGAATGGGTTGAATAAGAGGTATT |
| | | Escherichia_ | |
| | | phage_ | |
| | | vB_ | |
| | | EcoMACG.C40._ | |
| | | promoter | |
| 39 | prom26 | YP_ | GCCTTAAAAGCAACGGCACTATTTGCCATG |
| | | 007004486.1_ | CTAGGATTAGCGTTTGCTTTATCTCCACCA |
| | | phage_ | ATTGAAGCGAATGTCGATCCTCATTTTGAT |
| | | protein_ | AAATTTATGGAATCTGGTATTAGACACGTT |
| | | Escherichia_ | TATATGCTTTTTGAAAATAAAAGCGTAGAA |
| | | phage_ | TCATCTGAACAGTTCTATAGTTTTATGCGA |
| | | ime09._ | ACGACTTATAAAAATGACCCGTGCTCTTCC |
| | | promoter | GATTTTGAATGTATAGAGCGAGGCGCGGAG |
| | | | ATGGCACAATCATACGCTAGAATTATGAAC |
| | | | ATTAAATTGGAGACTGA |
| 40 | prom27 | YP_ | CGGTCTGTTATTTAAACGACGATTATGATT |
| | | 007004509.1_ | ATCTTGGCGTTTATAGTTTAAGTGATGCAC |
| | | predicted_ | GGTTTAAACGTAATTTACAAAAGTCAAATT |
| | | membrane_ | TATTTTATATTGATACTACGGTAAAATTTC |
| | | protein_ | AGGGCAAGAAATATTTCTTTACTCTTATAG |
| | | Escherichia_ | TTGATTCTGAAACGAAGCATGAGAATAAAC |
| | | phage_ | GTATTCTTAGTAAAAAGAATATCTTAACTA |
| | | ime09._ | TTGTTGATGATCTTTTTGATAAATTCGTAG |
| | | promoter | AAAATCCCAATTTTGAAAGCGATTTATTAC |
| | | | TAGAAAAATTTGTTAAGGAATGTAGAGAAT |
| 41 | prom28 | YP_ | TGAAATCGCCAAGAAAGTAGTTGAGTTAGA |
| | | 007004510.1_ | TGATGCTCGTCAAGAACTTGCGGTTAAATT |
| | | phage_ | GGAATATATCCGTGAAACTCGTGCAGCAAA |
| | | protein_ | TGCCCTTGGAATTAGTACTGCCGATGATGT |
| | | Escherichia_ | AGTTGAAATTGCAGCACTGACTAAGGTTGA |
| | | phage_ | TATTGAAGATACCCTTGCTCGAGTTGAAAC |
| | | ime09._ | CTTTAACGGTAATATTTCTGGGGTTGAAAC |
| | | promoter | TACCTCTGCCGATGTTCAGGAATACATTAA |
| | | | TTCTCTGAAATAATGATAAGGGGCTTCGGC |
| | | | CCCTTATACTTGGAGTAAATAGGAATGAAA |
| 42 | prom30 | YP_ | ACTGCTATAACTCCGCAAGAATACATGGCG |
| | | 009030656.1_ | TCTCTTAAAGAAAAATATAATCTTTCTGCA |
| | | DNA_ | ACAGAAACACTTTTCGATTTACCAGAAAAC |
| | | polymerase_._ | CTTCAACTAAAATTTCAGGTAGAATTTCAA |
| | | Escherichia_ | AAATTAGTTCACCCAGAACAAAAACACTTT |
| | | phage_ | ACTGCAGTCGTTAAGTCAATTAATGCAGAT |
| | | vB_ | GGATTGACAATTTTCACCCGACAAATAGTA |
| | | EcoM_ | CTAATTTAAGCAAGGGGCTTCGGCCCCTTA |
| | | 112._ | ATTGGAGTATAATATATCAAGAGCCTAATA |
| | | promoter | ACTCGGGCTATAAACTAAGGAATATCT |
| 43 | prom31 | YP_ | TTGAGCGAAAGTACTAAAGATCTGACTGAG |
| | | 009030781.1_ | TCTCAAAAAGAAAAAGTCTCTGCTCTGGTC |
| | | major_ | GAAGGTATGGATTATTCAGATGCATTCTCA |
| | | capsid_ | AGTAAATTGAGTGCAATCGTAGAAATGGTG |
| | | protein_ | AAGAAATCTAATAAAGATGAAAGCACTATT |
| | | Escherichia_ | ACTGAGAGTATAAATACTCCTGATACTGAA |
| | | phage_ | GCAGCCGGACTGAATTTCGTCACTGAAGCT |
| | | vBEcoM_ | GTAGAAGATAAATCTGCACAAGGTGCAGAA |
| | | 112._ | GATATTGTAAGTGTATATGCGAAAGTCGCA |
| | | promoter | TCTCGTTTCTAATTTTAAAGGTTAACACAA |
| 44 | prom33 | YP_ | TGTATTAGTAGGTCCTGTGACAGCTGTATC |
| | | 009148571.1_ | ATTTATAATCCTAATGATTATTGGAATAGT |
| | | transmembrane_ | TATAGATGTTACTACTGATATTGAATCAGA |
| | | region_ | CGCAGTATTTCTGTTAGTATTAATTCTTCC |
| | | domain. | ATTAGTAGTTCCATTTTTATTAGTACCTGT |
| | | containing_ | AAATTGGGTAGGATACTGGTATCAAGGAAG |
| | | protein_ | ACATTATCGTAAACGCGTATGCGAATGGAA |
| | | Escherichia_ | AGCTCAGTGTAAAAAGATTAAAAAGGAACA |
| | | phage_ | TCAGCTTAAACTTGCTGCGTATGAATTTAA |
| | | HY01._ | TGAAATTATGAAATTTGTTAAGGAATCACG |
| | | promoter | |
| 45 | prom34 | YP_ | CTATTGTAGTGTCAAGGTCAGGTGCTTATT |
| | | 009148631.1_ | CTGAAATGACTTATAGGAATGGCTATGAAG |
| | | putative_ | AAGCTATTCGTCTTCAAACTATGGCGCAAT |
| | | baseplate_ | ATGATGGCTATGCTAAATGTTCTACTGTCG |
| | | distal_ | GTAATTTTAACTTGACTCCTGGTGTTAAAA |
| | | hub_ | TTATTTTTAATGATAGTAAAAACCAATTTA |
| | | subunit_ | AAACAGAATTTTACGTTGATGAAGTTATCC |
| | | .Escherichia_ | ATGAATTATCAAATAATAATTCAGTAACTC |
| | | phage_ | ATCTATATATGTTCACTAATGCAACGAAAC |
| | | HY01.promoter | TGGAAACAATAGACCCAGTTAAGGTTAAAA |
| 46 | prom36 | YP_ | ATGCTCGTGAATTTCTTGACGAAGAAACCG |
| | | 009153641.1_ | GCGAGATGATTCGCGAAGAAAAATCTTGGC |
| | | head_ | GTGCAAAAGATACTAACTGCACTACATTCT |
| | | vertex_ | GGGGTCCTTTATTTAAGCATCAACCATTCC |
| | | assembly_ | GAGATGCTATTAAACGTGCTTATCAGTTAG |
| | | chaperone_ | GTGCTATTGATAGTAATGAAATTGTTGAAG |
| | | Yersiniaphage_ | CTGAAGTTGATGAATTGATTAACTCAAAGG |
| | | PST._ | TTGAAAAATTTAAATCTCCAGAAAGTAAAA |
| | | promoter | GTAAATCAGCTGCTGATTTAGAAACTGACC |
| | | | TCGAACAGTTAAGTGATATGGAAGAATTTA |
| 47 | prom37 | YP_ | TAAGTGATGAAGCTCATTTTAATTATCTGA |
| | | 009153650.1_ | TGGCTGCTGTTCCTCGGGGTAAAAGATATG |
| | | translational_ | GTAAATGGGCAAAACTGGTTGAAGATTCCA |
| | | repressor_ | CCGAAGTATTGATTATTAAGTTACTTGCTA |
| | | protein_ | AGCGGTATCAAGTTAATACAAATGATGCAA |
| | | Yersinia_ | TTAACTATAAATCAATTCTTACTAAAAATG |
| | | phage_ | GAAAACTATCTTTAGTATTAAAAGAACTAA |
| | | PST._ | AAGGTTTAGTCACGTATGATTTTTTGAAAG |
| | | promoter | AAGTGACTAAGAACGTAAAAGAACAGAAAC |
| | | | AACTCAAAAAACTAGCATTGGAATGGTAAA |
| 48 | prom38 | YP_ | ATCGTAATTAACGGTTTTAATAAAGTAGAA |
| | | 009153652.1_ | GATTCTGCTCTGACCCGTGTTAAATATTCT |
| | | clamp_ | TTGACTCTTGGTGATTATGATGGTGAAAAT |
| | | loader_ | ACATTTAATTTCATTATCAATATGGCAAAT |
| | | subunit.DNA_ | ATGAAAATGCAACCAGGAAATTATAAACTT |
| | | polymerase_ | CTGCTCTGGGCAAAAGGTAAACAAGGTGCT |
| | | accessory_ | GCTAAATTTGAAGGTGAACACGCGAATTAT |
| | | protein_ | GTGGTAGCTCTTGAAGCTGATTCTACCCAC |
| | | Yersinia_ | GATTTTTAATAAAGGGCTTCGGCCCTTTAT |
| | | phage_ | AATTTACACTAAAACTTGAATGAGGAAATT |
| | | PST._ | |
| | | promoter | |
| 49 | prom39 | YP_ | TTTCAGTTAAAAATCTGCATCACGTTGTTT |
| | | 009180682.1_ | TAGCACACGGTGTTAAATCTAAAATTATTG |
| | | ATP.dependent_ | TATTGCAAACAATAGGTCGCGTATTACGTA |
| | | DNA_ | AACATGGTTCTAAAACAATCGCAACAGTCT |
| | | helicase_ | GGGACCTCATAGATGACTGTGGTGTCAAGC |
| | | UvsW_ | CAAAATCTGCTAATACTAAAAAGAAATATG |
| | | .Escherichia_ | TTCACTTGAACTATCTTTTAAAACACGGCA |
| | | phage_ | TTGATCGTATTCAGCGCTACGCAGATGAAA |
| | | slur14.promoter | AATTTAATTACGTAATGAAAACAGTCAATT |
| | | | TATAAGGGCTTCGGCCCTTTGGAGAAAAAG |
| 50 | prom41 | YP_ | TATAATAAATTTTGGTCAGAAGACGAAGAA |
| | | 009210222.1_ | AAAGACCTTTTAAATCTTTTAGACTCTTTA |
| | | dCTPase_ | AATGACAGAGGAATAAAATTTGGACTGTCG |
| | | Escherichia_ | AATGTTTTAGAGCACCACGGAAAGGAAAAT |
| | | phage_ | ACTCTTCTTAAAGAATGGTCTAAAAAATAC |
| | | wV7._ | ACTGTTAAGCATCTTAATAAAAAATACGTC |
| | | Escherichia_ | TTTAACATATATCATTCCAAAGAAAAGAAT |
| | | phage_ | GGAACTGATGAAGTATATATTTTTAATTAA |
| | | slur02._ | TTGCTTATATATTCAAATGGTATAATTATT |
| | | promoter | TAACTTATTAATGAATTGAAAGGAAAAATA |
| 51 | prom42 | YP_ | CAAAGCATCACGCTTATGGAATGTTCCAAA |
| | | 009210302.1_ | ATTATTTGCCTACTATGCGAGCGAGAGTCA |
| | | valyl.tRNA_ | AGGAACTTGGTTATAATATGACCGATGCTG |
| | | synthetase_ | AAATAAAAAGAATGTTGAATAAACGGTCCA |
| | | modifier_ | ATTCAGCTTCCTGGGCGTACATTGAACTTT |
| | | Escherichia_ | CTTATTGGTTAAATATACATAAGGGCGATA |
| | | phage_ | TAAGAAAAGCAATATCCTCTTATAATTCGG |
| | | e11.2._ | GATGGAATGTTAAAGCTGGTTCTAAATATG |
| | | Escherichia_ | CTTCTGAAGTCCTAGAAAAGGCTAATTACC |
| | | phage_ | TTAAAAATAATAAACTTTTGGAAATAGTAA |
| | | slur02.promoter | |
| 52 | prom43 | YP_ | AATCAAAGAAACTGAATCAGCATTTCGTTT |
| | | 009277634.1_ | AGCTTTTGCCAAGGCACATTTCATTAAGAA |
| | | transcriptional_ | AGTAATTTCAGGTGAAATTGTTGTACAAGG |
| | | regulator_._ | TAAAACTCGCAAAGAACTGACCGAAGAACT |
| | | Shigella_ | TTCTAAAATTGATATGTATTCTTCTTATGT |
| | | phage_ | TGATAAACTAGTTGGAATGAACATTTTTCA |
| | | SHFML.11._ | TATGACTTCCGACGAAGCAAAGAAACTTGC |
| | | promoter | TGAAGAAGCTAAAGCCAAAAAAGAAGAAAA |
| | | | CGAATATTGGAAAACTACTGATGTAGTTAC |
| | | | AGAATACACCAAAGATTTAGAGGAAATCAA |
| 53 | prom45 | YP_ | GAAATAACTAAAGATCAGTTTTATCTTCTT |
| | | 009277656.1_ | CAAGATAAAGTAAGCGAAATTTATGAAATT |
| | | exonuclease_._ | GCTTATAGCAAAAATCGTGAAACTGTAAAA |
| | | Shigellaphage_ | ATAGAATCTAGTAAGTTGATGCTTCAATTA |
| | | SHFML._ | GAAGAAATTGAACGAGATTTAATTGCGTTA |
| | | 11._ | GAATTCTTTTGTGGTGAAGTGAAAACTGTT |
| | | promoter | ACAATCAATGATTATATTTTAGGCGAAATT |
| | | | AGCTATCTTTATAAGGCGATTATTA |
| 54 | prom46 | YP_ | TCGGAGGGGGAAGAAGAAAGAAAAGAACGT |
| | | 009277682.1_ | CTTTTTAATGAATCTCTTAAGATAATTAAA |
| | | DNA_ | TCTGCCATGGAAAATGTTATCCAGGAGATT |
| | | primase. | GTCATTAAACTAGAAGATGGTTCTACACAC |
| | | helicase. | ATTGTGTATGTGACAAAATTAGATTGGGTT |
| | | Shigella_ | GATGGAAAAGTCGTAATGGACTTTGCTGTT |
| | | phage_ | CTTGACCAAGAAAGAAAAGCTGAGTTAGCT |
| | | SHFML.11._ | CCTCATGTAGAAAAATGTATTACAATGCAA |
| | | promoter | CTACAAGATGCATTTAATAAAAGGTCAAAG |
| | | | AAAAAGTTTAAATTCTTTTAAGGAGTAAGT |
| 55 | prom47 | YP_ | GAATATTCAACTGGACAGCATTTATTAACT |
| | | 009277695.1_ | TTTCCTGAAATAAAACGATATATTCTGAGA |
| | | R | AATAATTTTTCTAATGAAGAGCATATAGTT |
| | | NA_ | ACTGAATCTATGCTTAGGAATGCATTTAAA |
| | | polymerase_._ | GCAGAATATACAAAAATAATGTCCAATAGA |
| | | Shigella_ | AATGAAGCTTGGACTGTTACTGATTATTAT |
| | | phage_ | GACTAAAGGTGTATT |
| | | SHFML.11._ | |
| | | promoter | |
| 56 | prom50 | YP_ | ATTTACGATAAAGTTAAGTCTATTAAAGAC |
| | | 009278976.1_ | CGTAGTGTTATTAAACGTGAAGTTGGTATT |
| | | tail_ | ATTGCTCAGGACCTTGAAAAGGAATTACCG |
| | | fibers_ | GAAGCTGTATCTAAAGTTGAAGTTGATGGA |
| | | protein_._ | TCTGATGTTCTGACAATTTCTAACTCTGCT |
| | | Shigella_ | GTGAATGCTCTTTTAATTAAGGCTATTCAG |
| | | phage_ | GAAATGAGTGAAGAAATTAAAGAATTGAAA |
| | | SHFML. | ACTCCTTTCTTTACTAAAATTGCTCGCAAA |
| | | 26._ | ATTAGTAAATATTTTAAATTCTAACAACAA |
| | | promoter | GGGGGAAATGCCCCCTTTGGAGTATAAATT |
| 57 | prom51 | YP_ | GAACTGTATAAGTTTAACCTATTTTTAGGT |
| | | 009279010.1_ | AAAACGGCAGAAACTTATAAAAATTGGAAC |
| | | exonuclease_._ | AAAGGCGGAAAAGCTCCATGGAGTGATTTT |
| | | Shigellaphage_ | TGGGATGCTAAATCACAGTTTAGTAAAGTG |
| | | SHFML.26._ | AAAGCACTTCCTGCATCAACATTCCATAAA |
| | | promoter | GCACAGGGCATGTCTGTAGACCGTGCTTTC |
| | | | ATTTATACGCCTTGTATTCATTATGCAGAT |
| | | | GCTGAATTGGCTCAACAACTTCTTTATGTT |
| | | | GGTGTTACCCGTGGTCGTTATGATGTGTTT |
| | | | TATGTATGATTAAATTTGAGGAAGCTATTC |
| 58 | prom52 | YP_ | AAAATTGTTATGGGGTGCTTAGCAGTTTGT |
| | | 009279041.1_ | TTAGTTGCATTAGCAGCAGTTCCATTTGTT |
| | | immunity_ | AGTGTTGAAAATGATACTCAACCTGTGATT |
| | | protein_._ | GAATCTAGCACCGTTATTCACACTAATGGT |
| | | Shigella_ | AAAATATCAGTTAAAATTGATGATAATCTT |
| | | phage_ | CATGTGAATACTAATGGAACGCTGGGTGTT |
| | | SHFML.26._ | CAATTAGGTAATATCTGTGTAAGCACTACT |
| | | promoter | GGAGTAATTACTACTTGTATTTGAGGAAAT |
| | | | TATT |
| 59 | prom53 | YP_ | ATGCTGGTATTTTATCACTGGTTACTAACG |
| | | 009281391.1_ | ATCGTGGTGCTATTGATGATGTTCTTGAGT |
| | | clamp_ | CTCTCAAAAATAAAGATGTTAAACAACTCA |
| | | loader_ | GAGCTTTAGCACCAAAATATGCAGCTGATT |
| | | subunit_ | ATTCGTGGTTCGTAGGTAAACTTGCCGAAG |
| | | Escherichia_ | AAATCTATTCACGTGTAACTCCGCAGAGTA |
| | | phage_ | TTATTCGTATGTACGAAATTGTCGGCGAAA |
| | | UFV.AREG1._ | ATAATCAGTATCATGGTATTGCAGCTAATA |
| | | promoter | CTGAATTGCATTTAGCTTATCTTTTCATTC |
| | | | AGTTAGCATGTGAAATGCAGTGGAAGTGAT |
| 60 | prom54 | YP_ | TAAAGAAATAGATGGTTATACTTATGATAT |
| | | 009281524.1_ | TAATGACGTTTATGTATGTCAAAGATTGGA |
| | | baseplate_ | ATTTCAATACCAAGGAAATACATTTTATTT |
| | | hub_ | TAGACCTCCTGGAAAATTTGAACAATTTTT |
| | | subunittail_ | AACTGTAAGCGATATGTTATCCAAATGCTT |
| | | length_ | GCTTAAGGTCAATGATGAAGTTAAAGAAAT |
| | | determinator_ | TAATTTTCTTGAGATGCCAGCATTTGTTTT |
| | | Escherichia_ | AAAATGGGCAAATGATATTTCTACAACTTT |
| | | phageUFV.AREG1._ | AGCAATTCCTGGCCCTAATGGTCCAATAAC |
| | | promoter | CGGAATTGGCAATATTATTGGATTATTTGA |
| 61 | prom55 | YP_ | ACCAGCAGTTAAAAAAGAACTTGCTTCTAG |
| | | 009281525.1_ | ATTTGCTAAAATTGATGCCACTTATCAAGA |
| | | baseplate_ | GCTTAAGAAAAATCAACCTGAGGCCAAACC |
| | | tail_ | TGAAACTTCTGCTAAATCACCAGAAGCAAA |
| | | tube_ | ACAGGTCCAGGTGATTGAAAAGAACAAAGC |
| | | cap.Escherichia_ | ACAACAAGCTCCTGTTCAACAAGCATCTCC |
| | | phageUFV.AREG1._ | TTCAATCAATAATACTAATAATGTTATTAA |
| | | promoter | GAAAAATACTGTCGTTCATAATATGACACC |
| | | | TGTCACAAGCACAACTGCTCCTGGTGTATT |
| | | | TGGTGCGACGGGAGTTAATTAAGGAATAAT |
| 62 | prom57 | YP_ | TCATATTGTAGCTAAGATGTGTAATCTTAT |
| | | 009284228.1_ | TCCGGGAGATTTGATATTTTCCGGTGGTAA |
| | | putative_ | TACTCATATCTATATGAATCACGTAGAACA |
| | | prohead_ | ATGTAAAGAAATTTTGCGTCGTGAACCTAA |
| | | corescaffold_ | AGAGCTTTGTGAACTGGTAATAGGCGGATT |
| | | protein_._ | GCCTTATAAATTCCGCTATCTTTCTACTAA |
| | | Escherichia_ | AGAACAATTGGAATACATTCTTAAACTCAG |
| | | phage_ | GCCTAAAGATTTCGTTCTCAAAGATTATCA |
| | | HY03._ | GTCCCACGGCGTCTTGAAAGGAAAAATGGC |
| | | promoter | GGTGTAATTTTAATTTAATTGTGAGGATTT |
| 63 | prom58 | YP_ | GCTTCCTGATGCGGTTGAGGAGATGAAAGT |
| | | 009288587.1_ | CTTTTTAGAAAATCAGCTTGCGAAATATGA |
| | | spanin_ | TTGCGATGTGTTCATTAATCAGACTCAACC |
| | | Rz_._ | TAATGTTCATATTAACAACTGTAAATGCTA |
| | | Shigella_ | TATCATCGTTAATCCTTTAACGGGAAAACA |
| | | phage_ | TCGTCTTGGAATTAGTAATCCAAATCGTAG |
| | | SHBML.50.1._ | CGCATCGGATATGGCAGAAGATGTTGAGGC |
| | | promoter | ATGCTTTAAAATTTCTAAATCTCCGGCTGA |
| | | | ACATCATATTTTAATTAACGGTCTTTCTCA |
| | | | AGACGATATTATAGAGGTTATTAAAACTTT |
| 64 | prom59 | YP_ | AATGACTTCAATTGAATTTACTAGTGCCGT |
| | | 009618969.1_ | TAGAATAGCTCTACAGGAAATGGTTGTAAA |
| | | short_ | ATTTATTGCAATTGATTCATTTGAAGACCA |
| | | tail_ | TCCTACCATAGGAAATAAAATACAAGTTAA |
| | | fiber_._ | GTATTTAGATAACCAAGAACATATCTTAGA |
| | | Shigella_ | ACAATACTCTGATAAAGGAATTACTTTCAA |
| | | phage_ | ACAGGAAATAATTTCTCCTTCTAAACCTGG |
| | | Sf21._ | GTATGGAACTTGGCAATTATTAGGTGCGCA |
| | | promoter | AACTGTTACGCTAGATAGTCACACACAACC |
| | | | TACAGTATTTTATTATTTTGAGAGAGTAGC |
| 65 | prom60 | YP_ | TTGCTAATAATAAAGATAAAGTTCTGTATC |
| | | 009625198.1_ | AGTCCTGTCATATTCTTCAGAAAAAAGGAC |
| | | putative_ | TATACTATATCGTTCATTTTAAAGAAATGC |
| | | protein_ | TTCGTATGGATGGTCGCCAAGTTGAAATGA |
| | | ORF43.1_ | CAGAAGAAGATGAAGTTCGTCGTGATTCGA |
| | | Enterobacteria_ | TTGCATGGCTGTTAGAAGATTGGGGACTGA |
| | | phage_ | TTGAAATCGTTCCTGGTCAAAGAACTTTTA |
| | | ime09._._ | TGAAAGATTTAACTAATAATTTCCGAGTTA |
| | | Escherichia_ | TTTCTTTTAAACAAAAACATGAATGGAAAC |
| | | phage_ | TCGTTCCTAAATATACGATTGGTAATTAAT |
| | | slur03._ | |
| | | promoter | |
| 66 | prom61 | YP_ | CTCGGCTTTCTAAATCAAAACAAACCTCTG |
| | | 009625232.1_ | ACGTTAAGCTAACGATTGTAGCACTCAAAG |
| | | putative_ | CTCGTATTGATGGTTCTCGTATAGCAGAAG |
| | | phage_ | GCGCTGAAGTTGTTAAATTGAACGTTCTTC |
| | | protein. | TTAAAGGCTCTGATTGGAAAACTGTGAAAA |
| | | Escherichia_ | AGTTGTCAGAAGCAGAAATGCAATATGATA |
| | | phagewV7._ | TGTGTGATAAAATTATTCAAGGTGTAGAGC |
| | | Escherichia_ | GGTATCAAAACTTGTCTTTTATTGATAAAC |
| | | phage_ | TTAAACTGAAAAGAGGATACCCGTTAAATT |
| | | slur03._ | GTTCAATTTTTAAACTTATCCGAGGATAAT |
| | | promoter | |

### Example 24: Delivery of Biotherapeutic Proteins to Inflamed Areas of Murine Gut

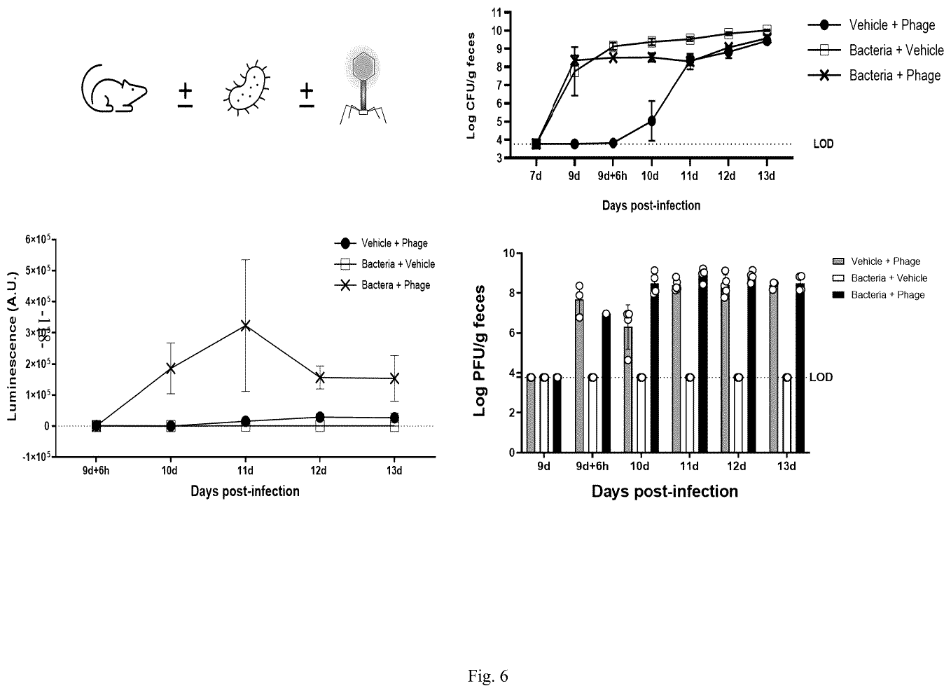

FIG. 6 shows that nLuc expression from a phage genome in vivo produces detectable luminescent signal. E. coli-targeting phage p00ex was engineered with nLuc. Mice were infected with an E. coli strain that supports the amplification of p00ex by oral gavage (a control group of mice were gavaged with buffer rather than E. coli). Mice were subsequently treated with either nLuc-engineered phage or buffer by oral gavage. Feces samples were collected daily and nLuc activity in the feces was measured (bottom left). Colony-forming units (CFU; top right) and plaque-forming units (PFU; bottom right) were also measured from the same fecal samples. The data shows that feces from mice treated with bacteria and nLuc-phage produce a higher luminescent signal than those treated with bacteria alone or phage alone. Notably, there is some luminescent signal from the group treated with phage alone. This may be due to a contamination event during the study, or may be due to the presence of endogenous E. coli in the mouse microbiome that can support the amplification of p00ex. CFU counts in the feces of the phage-only-treated mice start increasing in a similar timeframe to the luminescence, which supports the idea that the presence of a bacterial host is required in order to support protein expression from the engineered phage.

### Example 25: Phages Expressing Immune Modifiers are Active Against their Biological Target when Released from Phage-Infected Cells

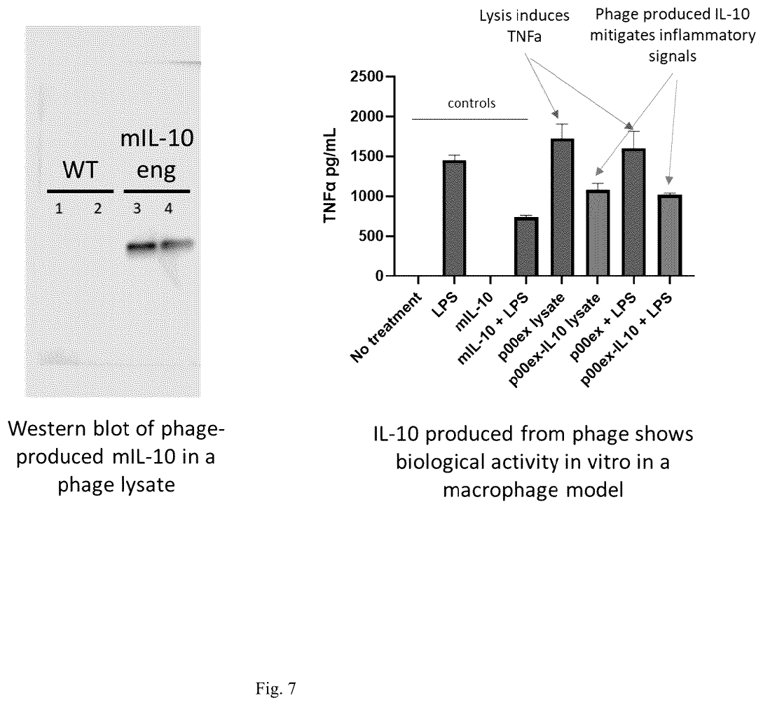

The E. coli-targeting phage p00ex was engineered to express mouse IL-10 (mIL-10). The phage was then used to infect a bacterial host strain b004531, which produced mIL-10 during infection. As shown in FIG. 7, the resulting phage lysate, as well as lysate from a wild type phage control, was run on a gel and subjected to Western blotting with anti-mIL-10 antibodies (left). The mIL-10 is clearly detectable in the lysate from the engineered phage. Next, the lysates from the wild type and engineered phages were used in a bone marrow-derived macrophage (BMDM) assay to determine whether the phage-produced mIL-10 was biologically active (FIG. 7, right). In this assay, macrophages are stimulated to produce the inflammatory molecule TNFα when exposed to lipopolysaccharide (LPS), a constituent of the bacterial cell membrane. The plot on the right shows that in the absence of any treatment, no TNFα is produced. When the BMDMs are treated with purified commercial LPS, a high level of TNFα is produced. Purified commercial mIL-10 alone does not lead to TNFα production. However, treating with purified LPS and purified mIL-10 results in a decreased level of TNFα compared to treatment with LPS alone, showing that the assay is working. When the BMDMs are treated with lysate from wild type p00ex, high levels of TNFα are produced due to residual LPS in the phage lysate (left over from when the cells burst after phage infection). Treatment with lysate from mIL-10-expressing phage results in a decreased level of TNFα relative to treatment with wild type phage lysate, showing that the mIL-10 present in the lysate is able to ameliorate the effect of the LPS that is also present in the phage lysate. Addition of LPS with phage lysate has no effect on TNFα level, presumably because the amount of LPS in the phage lysate has already saturated the system.

### Example 26: Anti-TNFα Nanobodies Produced from Phage Block L-Cell TNFα Cytotoxicity in a Dose-Dependent Manner

The E. coli targeting bacteriophage p00ex was engineered with two previously published anti-TNFα nanobodies, 5M2M and 5M2I (Beirnaert, 2017). To functionally measure the activity of the phage-encoded nanobodies an established TNFα cytotoxicity was used in which L929 murine fibroblasts (L-cells) are exposed to human TNFα in the presence of actinomycin D (Alexander, 1987; Ratter, 1999). Phage-derived nanobodies were prepared by clarifying a phage lysate through size-exclusion, then the clarified phage lysates containing the specified nanobodies were added in a 1:10 dilution series because the concentration of the nanobodies was unknown. The no treatment condition is the full viability control. Humira was used to ensure TNFα inhibition could be achieved and a phage-derived anti-GFP nanobody was used to ensure the effects of the phage-encoded anti-TNF a lysates were specific. After an 18h exposure remaining cell viability is monitored by a standard MTT assay in which more viable cells have a higher absorbance at 590 nm. The engineered Phage are depicted in Table 3.

TABLE 3
Engineered phage

| Phage | | | Deletion/insertion | Pro- |
|---|---|---|---|---|
| ID | Insert | Deletion/insertion | site(s) | moter |
| p00exe112 | anti-TNFa | insertion | r3 site | r3 |
| | nanobody | | | |
| | 5M2M_J | | | |
| p00exe113 | anti-TNFa | insertion | r3 site | r3 |
| | nanobody | | | |
| | 5M21_L | | | |

TABLE 4
Sequences

| | SEQ | |
|---|---|---|
| | ID | |
| Name | NO | Sequence |
| r3 | 86 | TATAAATAATAATATGAATT |
| | | GGGTGTCGGAATAATAAGTT |
| | | AACCGAACAATTCTATGTGG |
| | | TAGTCTACAACTGAGAGATC |
| | | TGTCGAAAGAAGATGAAATT |
| | | CAGAAGAACGTGACTACCGA |
| | | GTTTTAATCTCTAACGAGAA |
| | | TTTTTAA |
| anti-TNFa | 87 | ATGGTGCAACTGCAGGAATC |
| 5M2M_J | | AGGAGGAGGTCTTGTCCAGC |
| | | CAGGCGGCTCGTTACGCTTA |
| | | TCCTGCGCAGCGTCCGGGCG |
| | | CACCTTCAGCGATCACAGTG |
| | | GATATACATACACGATTGGT |
| | | TGGTTCCGCCAGGCTCCCGG |
| | | GAAAGAACGTGAGTTTGTCG |
| | | CGCGTATTTACTGGTCATCA |
| | | GGCAACACTTACTATGCTGA |
| | | TTCTGTTAAAGGTCGTTTCG |
| | | CTATTAGCCGCGATATTGCT |
| | | AAGAATACCGTGGACTTGAC |
| | | GATGAATAATTTAGAACCCG |
| | | AAGATACAGCGGTTTACTAC |
| | | TGTGCGGCTCGTGATGGAAT |
| | | CCCCACAAGTCGCTCGGTGG |
| | | AAAGTTATAACTATTGGGGA |
| | | CAAGGGACTCAAGTGACAGT |
| | | GTCTTCTTAA |
| anti-TNFa | 88 | ATGGTCCAACTGGTCGAGTC |
| 5M21_L | | CGGAGGCGGCTTGGTACAAG |
| | | CGGGCGGTTCGCTTTCCTTA |
| | | TCGTGTAGTGCCTCGGGTCG |
| | | CTCTTTGAGTAACTACTATA |
| | | TGGGCTGGTTTCGCCAAGCA |
| | | CCCGGTAAAGAACGCGAGCT |
| | | TCTTGGAAATATCAGTTGGC |
| | | GTGGCTATAACATCTATTAT |
| | | AAGGATAGCGTCAAGGGGCG |
| | | CTTCACCATTTCGCGTGATG |
| | | ATGCAAAGAATACCATTTAT |
| | | TTGCAGATGAATCGTTTGAA |
| | | ACCTGAGGACACAGCAGTTT |
| | | ACTACTGTGCCGCAAGTATT |
| | | TTACCTTTGAGTGATGACCC |
| | | AGGTTGGAACACTTACTGGG |
| | | GACAAGGTACCCAGGTCACC |
| | | GTTAGCTGA |
| anti-TNFa | 89 | MVQLQESGGGLVQPGGSLRL |
| 5M2M_J | | SCAASGRTFSDHSGYTYTIG |
| | | WFRQAPGKEREFVARIYWSS |
| | | GNTYYADSVKGRFAISRDIA |
| | | KNTVDLTMNNLEPEDTAVYY |
| | | CAARDGIPTSRSVESYNYWG |
| | | QGTQVTVSS* |
| anti-TNFa | 90 | MVQLVESGGGLVQAGGSLSL |
| 5M21_L | | SCSASGRSLSNYYMGWFRQA |
| | | PGKERELLGNISWRGYNIYY |
| | | KDSVKGRFTISRDDAKNTIY |
| | | LQMNRLKPEDTAVYYCAASI |
| | | LPLSDDPGWNTYWGQGTQVT |
| | | VS* |

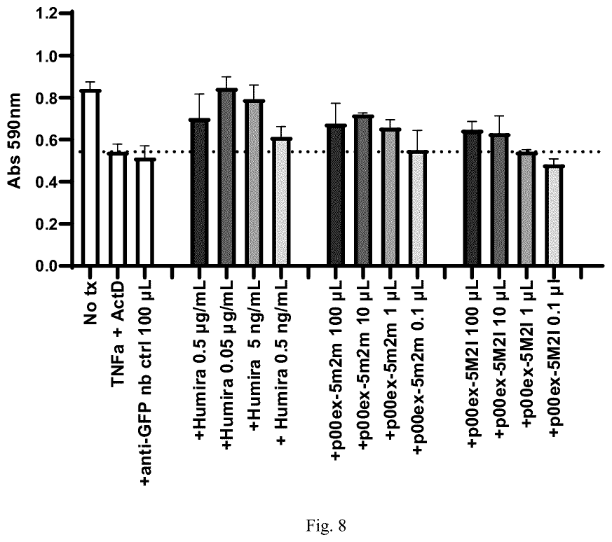

Results are depicted in FIG. 8. There was a measurable increase in cell viability in the presence of both human TNFα monoclonal antibody Adalimumab (Humira) and the phage-derived anti-TNFα nanobodies 5M2M and 5M2I. The anti-GFP nanobody was ineffective and was as cytotoxic as the TNFα-Actinomycin D. These results demonstrate that the phage lysates alone are insufficient to prevent L-cell cytotoxicity, but the addition of the anti-TNFα nanobodies specifically blocked the cytotoxic effects of TNFα in a dose-dependent manner.

The assay was run in biological triplicate, with the error bars displaying standard deviation. The dotted line is set at the mean of the TNFα-Actinomycin D cell viability to easily show where the signal is above the line as more viable than the untreated control.

### Example 27: Lokta-Volterra Dynamics to Deliver Long-Term Expression of an Engineered Payload

A population of bacteriophage is engineered to include a nucleic acid encoding for a therapeutic peptide. The bacteriophages are administered to a subject and targets bacterial cells in the target. The population of bacteriophage is administered to the subject at a low dose so that not all bacterial cells are infected.

Once a bacteriophage has entered a bacterial cell, the bacteriophage replicates and the therapeutic peptide is produced. The bacterial cell lyses and the therapeutic peptide is release to the subject. A population of newly produced bacteriophage continue to target bacterial cells and the reproduction cycle continues.

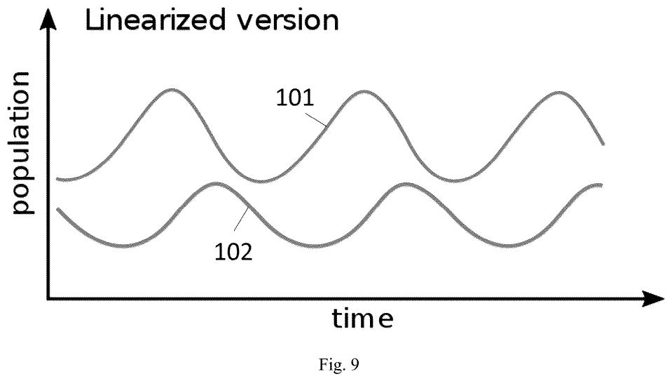

The population levels of the bacteriophage and the bacterial cells are depicted in FIG. 9. As the numbers of bacterial cells increases, the number of bacteriophage that can be supported similarly rises. However, once the bacteriophage population reaches too high of an abundance, there is a crash in the bacterial cell population as they are consumed. Due to the sudden reduction in available prey resources, the bacteriophage population also experiences a substantial decline. This cycle then repeats indefinitely. A deliberate co-existence of the phage and target bacterial populations is established in order to achieve continual, long-term, site-localized expression of the therapeutic peptide.

### Example 28: TNFRI Engineered Phage

The E. coli targeting bacteriophage p00ex was engineered to express TNFRI. The engineered phage are described in Tables 5-6.

TABLE 5
Engineered phage

| Phage | | | Deletion/insertion | Pro- |
|---|---|---|---|---|
| ID | Insert | Deletion/insertion? | site(s) | moter |
| p00exe114 | TNFa 206- | insertion | r3 site | r3 |
| | 211 peptide | | | |

TABLE 6
Sequences

| | SEQ ID | |
|---|---|---|
| Name | NO | Sequence |
| TNFa 206- | 91 | ATGGAGGATTCCGGAACCACCTGA |
| 211 peptide | | |
| TNFa 206- | 92 | MEDSGTT* |
| 211 peptide | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-109. (canceled)

110. A lytic bacteriophage comprising a nucleic acid encoding a nanobody.

111. The lytic bacteriophage of claim 110, wherein the lytic bacteriophage comprises a nucleic acid encoding a lytic gene sequence.

112. A method of producing a nanobody in a subject, the method comprising:
(a) contacting the subject with the lytic bacteriophage of claim 110;
(b) producing the nanobody within one or more of a plurality of target bacterial cells present in the subject; and
(c) releasing the nanobody by lysis of the one or more of a plurality of target bacterial cells.

113. The method of claim 112, wherein the lytic bacteriophage and the one or more of a plurality of target bacterial cells co-exist in the subject for at least about 1 day after the contacting.

114. The method of claim 112, wherein the nanobody is released for at least about 1 day after the contacting.

115. The method of claim 112, wherein a therapeutically effective amount of the nanobody is produced without eliminating the one or more a plurality of target bacterial cells.

116. The method of claim 112, wherein the lytic bacteriophage does not incorporate into the subject genome or the one or more of a plurality of target bacterial cells genome.

117. The method of claim 112, wherein the nanobody affects TNF-α signaling, IL-1 signaling, IL-6 signaling, IL-4 signaling, IL-13 signaling, IL-2 signaling, TGF-β signaling, EGF signaling, HGH signaling, IGF signaling, NGF signaling, ROS1 signaling, ALK signaling, IFNγ signaling, IDO signaling, PD-1 signaling, PD-L1 signaling, CTLA-4 signaling, LAG-3 signaling, VISTA signaling, TIM-3 signaling, MMP signaling, VEGF signaling, or Wnt signaling, or two or more thereof.

118. The method of claim 112, wherein the nanobody binds to a pro-inflammatory peptide.

119. The method of claim 118, wherein the nanobody binds to TNF-α, IL12, IL-17, IL-17A, IL-17R, IL-23, IL-23A, MAdCAM-1, α4β7-integrin, α4β1-integrin, or αEβ7-integrin, or two or more thereof.

120. The method of claim 112, wherein the subject has inflammation or cancer.

121. The method of claim 120, wherein a therapeutically effective amount of the nanobody is produced to treat inflammation or cancer in the subject.

122. A lytic bacteriophage comprising a promoter operably linked to a nucleic acid encoding a therapeutic peptide, wherein the promoter is at least 80% or 90% identical to any one of SEQ ID NOS: 1-66 or 74-81.

123. The lytic bacteriophage of claim 122, wherein the promoter is a bacterial promoter.

124. The lytic bacteriophage of claim 122, wherein the promoter is a phage promoter.

125. A method of producing a therapeutic peptide in a subject, the method comprising:
(a) contacting the subject with the lytic bacteriophage of claim 122;
(b) producing the therapeutic peptide within one or more of a plurality of target bacterial cells present in the subject; and
(c) releasing the therapeutic peptide by lysis of the one or more of a plurality of target bacterial cells.

126. A lytic bacteriophage comprising a nucleic acid encoding an anti-inflammatory interleukin.

127. The lytic bacteriophage of claim 126, wherein the anti-inflammatory interleukin comprises IL-4, IL-6, IL-9, IL-10, IL-11, IL-13, IL-10, IL-27, IL-35, IL-37, or two or more thereof.

128. A method of producing an anti-inflammatory interleukin in a subject, the method comprising:
(a) contacting the subject with the lytic bacteriophage of claim 126;
(b) producing the anti-inflammatory interleukin within one or more of a plurality of target bacterial cells present in the subject; and
(c) releasing the anti-inflammatory interleukin by lysis of the one or more of a plurality of target bacterial cells.

129. The method of claim 128, wherein the anti-inflammatory interleukin interacts with an immune cell of the subject.
